(19) United States
(12) Patent Application Publication
(10) Pub. No.: US 2023/0038627 A1
(43) Pub. Date: Feb. 9, 2023

(54) PARENTERAL LYSOPHOSPHATIDYLCHOLINE FORMULATIONS SUCH AS LPC-DHA, LPC-EPA AND THEIR USE IN THERAPY

(71) Applicant: AKER BIOMARINE ANTARCTIC AS, Stamsund (NO)

(72) Inventors: Lotte SKOLEM, Stamsund (NO); Finn MYHREN, Stamsund (NO); Nils HOEM, Stamsund (NO); Petter-Arnt HALS, Stamsund (NO); Andreas BERG STORSVE, Stamsund (NO); Armend Gazmeno HÅTI, Stamsund (NO)

(21) Appl. No.: 17/620,938

(22) PCT Filed: Jun. 21, 2020
(86) PCT No.: PCT/EP2020/067263

§ 371 Date: Dec. 20, 2021

Related U.S. Application Data
(60) Provisional application No. 62/864,073, filed on Jun. 20, 2019.

(51) Int. Cl. A61K 31/202; A61K 31/66; A61K 9/00; A61P 25/00

(52) U.S. Cl. CPC A61K 31/202; A61K 31/66; A61K 9/0019; A61P 25/00

(57) ABSTRACT

The present invention relates to pharmaceutical formulations of phospholipids, and in particular pharmaceutical formulations which are administered intravascularly such as intravenously. In particular, the present invention provides pharmaceutical compositions for intravascular administration comprising phosphatidylcholine derived compounds carrying an omega-3 fatty acid for use in prophylaxis or therapy.

### FIELD OF THE INVENTION

The present invention relates to pharmaceutical formulations of phospholipids, and in particular pharmaceutical formulations which are administered intravascularly such as intravenously. In particular, the present invention provides pharmaceutical compositions for intravascular administration comprising phosphatidylcholine derived compounds carrying an omega-3 fatty acid for use in prophylaxis or therapy.

### BACKGROUND OF THE INVENTION

Docosahexaenoic acid (DHA), an essential omega 3 fatty acid, is uniquely concentrated in the brain, nervous tissues and retina, and is essential for the normal neurological development and function. The deficiency of DHA is associated with several neurological disorders, including Alzheimer's, Parkinson's, schizophrenia, and depression.

Unlike liver, the brain cannot efficiently convert dietary alpha linolenic acid (18:3, n-3) to DHA and is almost completely dependent upon the uptake of preformed DHA from the plasma. However, dietary supplementation with the currently available preparations of DHA such as fish oil, algal DHA, DHA-enriched egg phospholipids and sardines does not appreciably increase brain DHA levels in adult mammals, although peripheral tissues are enriched with DHA under the same conditions.

One possible explanation for this is that DHA from the above supplements is hydrolyzed to free DHA by the pancreatic enzymes and absorbed as triacylglycerol (TAG) in chylomicrons, whereas the brain uniquely takes up DHA in the form of lysophosphatidylcholine (LPC). The recent demonstration of a transporter (Mfsd2a) at the blood brain barrier (Nature. 2014 May 22; 509(7501):503-6.), which specifically transports LPC-DHA but not free DHA, further supports this mechanism. It has therefore been hypothesized that it is necessary to increase the levels of LPC-DHA in plasma for an efficient enrichment of brain DHA.

In order to increase the levels of LPC-DHA in plasma, it has recently been proposed that dietary DHA provided in the sn-1 position of phosphatidylcholine (PC) or in the form of LPC in the diet, may escape the hydrolysis by pancreatic PLA2, and may be absorbed as PC-DHA. Furthermore, it has been hypothesized that PC-DHA is more likely to be taken up by the brain after conversion to LPC-DHA in plasma or liver by the phospholipases, compared to TAG-DHA, which requires extensive metabolic transformations in the liver in order to form LPC-DHA.

A follow-up study in the prior art confirmed the above hypothesis in that the amount of DHA that is absorbed in the form of phospholipid indeed can be increased by up to 5-fold by providing the DHA in the form of LPC, relative to free DHA. It was also found that incorporation of DHA into intestine-derived HDL was increased 2- fold during the absorption of LPC-DHA, compared to the absorption of free DHA.

In the above study it was also tested whether the increased DHA absorption in the phospholipid form not only increases brain DHA levels but also improves cognition and memory in normal adult mice. The incorporation of dietary free DHA and LPC-DHA into the brain and other tissues was compared following daily gavage of the compounds in a corn oil vehicle for 30 days. The results showed that the DHA content of most regions of the brain is more than doubled by feeding LPC-DHA, but not by feeding free DHA, which however enriched other tissues. Furthermore, the mice treated with LPC-DHA also showed a remarkable enhancement of spatial learning and memory in the Morris water maze test. These studies were the first to demonstrate a targeted enrichment of brain DHA through diet leading to a functional improvement in memory in normal adult mice, and the question is whether this treatment strategy also may have the potential for the prophylaxis and treatment of other neurological disorders that are associated with low cerebral DHA levels or which would benefit from increased levels of cerebral DHA levels.

Traumatic brain injury (TBI) is a neurological disorder with major cause of death and permanent disability for people under the age of 45 that may benefit from increased levels of cerebral DHA levels. This injury occurs frequently in military personnel and professional athletes, leading to loss of limb function, speech impairment, memory disturbances, and emotional responses. It is a multifaceted disease with prolonged secondary pathogenesis of excitotoxicity, oxidative stress, inflammation, and long-lasting adverse neurological sequelae such as secondary epilepsy, chronic headaches, post-traumatic stress disorder, neurocognitive deficit, as well as neurodegenerative diseases of Alzheimer's disease or Parkinsonism. Current TBI treatments focus on the management of intracranial pressure, the prevention and treatment of hypotension, and adequate ventilation, but no specific medical treatment is provided specifically for neuroprotection and recovery.

Recent animal studies have demonstrated that dietary supplementation with DHA either before or after TBI improves functional outcomes (Brain Injury. ASN Neuro 7, 1-15, 2015). Mechanistic investigations suggest that DHA influences multiple aspects of the pathologic molecular signaling cascade including decreased neuroinflammation and oxidative stress, neurotrophic support, and the activation of cell survival pathways. Increased plasma level of DHA has been observed at day 1 but decreased 3 days after injury (J Neurosci. 30, 3220-3226, 2010).

In view of the above it is clear that there are a number of different conditions, in particular neurological conditions and TBI in particular, which may benefit from increased cerebral DHA levels. Furthermore, it has previously been hypothesized that an increase in the plasma levels of LPC-DHA is a prerequisite for an efficient enrichment of cerebral DHA levels.

Thus, there is a need in the art for means to increase the levels of LPC-DHA in serum.

LPC is found only in trace amounts in most animal tissues, since greater concentrations are known to facilitate disruption of cell membranes. In order to reduce the effective concentration to a safe level in plasma, LPC molecules are commonly bound to albumin and lipoproteins in serum.

Thus, any increased levels of LPC-DHA in plasma should preferably be kept at a safe level to avoid disruption of cell membranes and other potential side effects.

Furthermore, it has previously been suggested that dietary DHA provided in the sn-1 position of phosphatidylcholine (PC), or in the form of LPC in the diet, may be an effective way of increasing the levels of LPC-DHA in serum. However, in case of a neurological condition, such as TBI, the time from intake of dietary DHA until a raise in the levels of LPC-DHA in serum may be of outermost importance.

Thus, there is an urgent need in the art for means to increase the levels of LPC-DHA in plasma at a fast rate.

Another issue that should be considered is the need of a continuous supply of DHA into the brain. It is well known that administered drugs typically are removed from the circulation by various elimination processes, and such processes for elimination of LPC-DHA may of course represent a potential problem that needs to be solved.

Thus, there is a need in the art for means to increase the levels of LPC-DHA in serum which also ensures a high level in plasma for a prolonged period of time.

Means which solves most or all of the above-mentioned problems may have the potential of being a prophylactic and/or therapeutic agent for a number of different conditions which may benefit from increased DHA levels in the brain. Examples of such conditions being neurological conditions, such as depression, Schizophrenia, Alzheimer's disease, Parkinson's disease or traumatic brain injury. A non-limiting list of other conditions that may benefit from increased DHA levels in the brain are post-traumatic stress disorder (PTSD) and anxiety.

The above discussion has been focused on the levels of DHA in the brain. However, the person skilled in the art will be aware of other omega-3 fatty acids that also are assumed to be important for normal neurological development and function of the brain, either directly or indirectly in the sense that they may be converted into omega-3 fatty acids which are important for normal neurological development and function. A non-limiting list of such omega-3 fatty acids that are assumed to influence neurological development and function in the brain is docosapentaenoic acid (n3-DPA), stearidonic acid (SDA) and Eicosapentaenoic acid (EPA). α-linolenic acid (ALA) is another omega-3 fatty acid which may influence neurological development and function in the brain. The benefit of having an increased level of LPC-DHA in serum is therefore equally relevant in respect of LPC-DPA, LPC-SDA, LPC-EPA and LPC-ALA; and in particular LPC-DPA, LPC-SDA and LPC-EPA.

Further, there has been some discussion in the art whether uptake of the omega-3 fatty acids into the brain may be affected by the localization of the omega-3 fatty acid in the LPC molecule, i.e. whether the omega-3 fatty acid is in the sn1 (2-LPC) or sn2 (1-LPC) position of the LPC molecule. However, the skilled person will be aware that there is an equilibrium between these two LPC forms, where an equilibrium mixture of 90% 2-lysoPC and 10% 1-lysoPC typically is obtained with a half-time of about 10 minutes under physiological conditions.

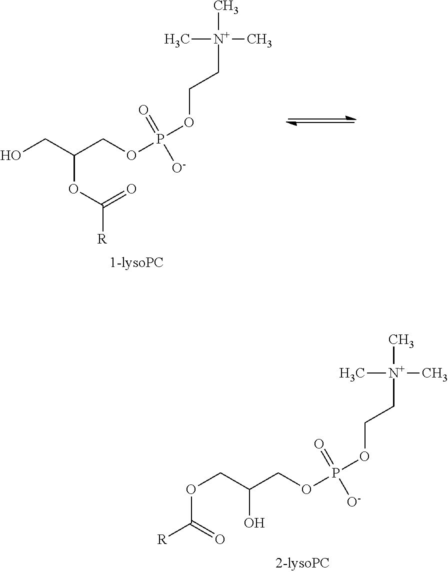

In those cases where it would be of interest to have a ratio between 1-LPC and 2-LPC which is different from the normal equilibrium under physiological conditions, or to have a composition with only LPC-1 or LPC-2, there are compounds that have been developed which blocks migration of the acyl group from the sn-1 position of the glycerol backbone to the sn-2 position and vice versa. This has been achieved by reacting the OH-group on the glycerol backbone with a protecting group such as e.g. O—CO—CH₃ (WO2018162617, WO2008068413).

### SUMMARY OF THE INVENTION

The present inventors have solved the above-mentioned needs by providing pharmaceutical compositions for intravascular administration comprising phosphatidylcholine derived compounds carrying an omega-3 fatty acid. The pharmaceutical composition has been designed for intravascular administration, such as intravenous administration, and was surprisingly shown to cause a significant increase in the uptake of the omega-3 fatty acid into the brain at a fast rate and for a prolonged period of time.

Thus, a first aspect the present invention relates to a pharmaceutical composition suitable for intravascular administration, such as intravenous administration; the pharmaceutical composition comprising one or more active components and one or more pharmaceutically acceptable excipients; the one or more active components being selected from the group consisting of a compound according to any one of formula 1 to 8, or a pharmaceutically acceptable salt thereof, and any combination thereof

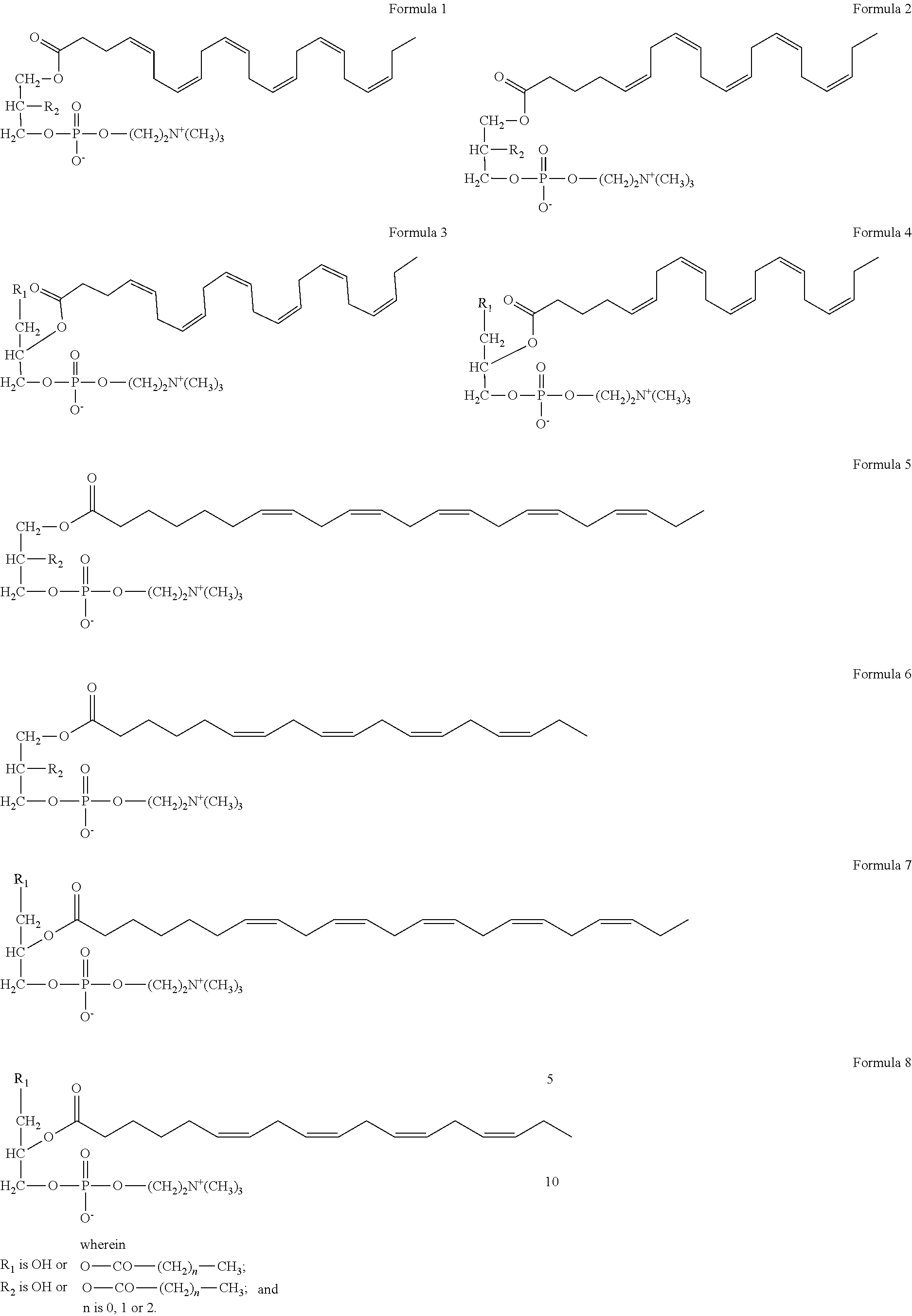

In one embodiment according to the present invention, the intravascular administration is intravenous administration. Intravenous administration may be conducted by injections, e.g. with a syringe at higher pressures, or by infusions, e.g. using only the pressure supplied by gravity. In one embodiment, the intravenous administration is conducted by one or more injections, preferably less than 5 injections, more preferably less than 3 injections and most preferably by 2 injections or 1 injection.

A preferred embodiment according to the first aspect of the present invention relates to a pharmaceutical composition suitable for intravascular administration, such as intravenous administration; the pharmaceutical composition comprising i) LPC-EPA, or a pharmaceutically acceptable salt thereof; and ii) LPC-DHA or a pharmaceutically acceptable salt thereof. Preferably the LPC-EPA and LPC-DHA constitutes from 10 to 99% by dry-weight or by weight of the pharmaceutical composition, such as from 15 to 99% by dry-weight or by weight of the pharmaceutical composition, from 20 to 99% by dry-weight or by weight of the pharmaceutical composition, from 25 to 99%, such as about 27%, by dry-weight or by weight of the pharmaceutical composition, from 35 to 99% by dry-weight or by weight of the pharmaceutical composition, from 55 to 99% by dry-weight or by weight of the pharmaceutical composition, from 75 to 99% by dry-weight or by weight of the pharmaceutical composition and most preferably from 80 to 99%, such as 85 to 95% (e.g. about 89%), by dry-weight or by weight of the pharmaceutical composition.

In another embodiment the intravenous administration may be conducted by infusion, such as infusion over a prolonged time. In one preferred embodiment, prolonged time is more than 6 hours, such as more than 12 hours, more than 24 hours or 48 hours or more.

In one embodiment, the one or more active components is a compound according to formula 1, wherein R₂ is OH or O—CO—(CH₂)ₙ—CH₃; and n is 0, 1 or 2.

In another embodiment according to the present invention, the one or more active components is a compound according to formula 2, wherein R₂ is OH or O—CO—(CH₂)ₙ—CH₃; and n is 0, 1 or 2.

In another embodiment according to the present invention, the one or more active components is a compound according to formula 3, wherein R₁ is OH or O—CO—(CH₂)ₙ—CH₃; and n is 0, 1 or 2.

In yet another embodiment according to the present invention, the one or more active components is a compound according to formula 4, wherein R₁ is OH or O—CO—(CH₂)ₙ—CH₃; and n is 0, 1 or 2.

In yet another embodiment according to the present invention, the one or more active components is a compound according to formula 5, wherein R₂ is OH or O—CO—(CH₂)ₙ—CH₃; and n is 0, 1 or 2.

In yet another embodiment according to the present invention, the one or more active components is a compound according to formula 6, wherein R₂ is OH or O—CO—(CH₂)ₙ—CH₃; and n is 0, 1 or 2.

In yet another embodiment according to the present invention, the one or more active components is a compound according to formula 7, wherein R₁ is OH or O—CO—(CH₂)ₙ—CH₃; and n is 0, 1 or 2.

In yet another embodiment according to the present invention, the one or more active components is a compound according to formula 8, wherein R₁ is OH or O—CO—(CH₂)ₙ—CH₃; and n is 0, 1 or 2.

In yet another embodiment according to the present invention, the one or more active components is a combination of two or more of the above mentioned active components.

In yet another embodiment according to the present invention, the one or more active components is a combination of three, four, five or more of the above mentioned active components.

One embodiment according to the first aspect of the present invention relates to a pharmaceutical composition according to the first aspect of the present invention, with the proviso that: if the pharmaceutical composition comprises i) a compound according to formula 1, wherein R₂ is OH, or a pharmaceutically acceptable salt thereof; and/or ii) a compound according to formula 3, wherein R₁ is OH, or a pharmaceutically acceptable salt thereof; then the pharmaceutical composition further comprises at least one of the other active components referred to in the first aspect of the present invention.

The expression “at least one of the other active components” recited above refers to at least one active component different from i) a compound according to formula 1, wherein R₂ is OH, or a pharmaceutically acceptable salt thereof; and different from ii) a compound according to formula 3, wherein R₁ is OH, or a pharmaceutically acceptable salt thereof.

In yet another embodiment according to the present invention, the one or more active components is i) a compound according to formula 1, or a pharmaceutically acceptable salt thereof; ii) a compound according to formula 2 or a pharmaceutically acceptable salt thereof; iii) a compound according to formula 3 or a pharmaceutically acceptable salt thereof; and iv) a compound according to formula 4 or a pharmaceutically acceptable salt thereof.

In yet another embodiment according to the present invention, the one or more active components is i) a compound according to formula 5, or a pharmaceutically acceptable salt thereof; ii) a compound according to formula 6 or a pharmaceutically acceptable salt thereof; iii) a compound according to formula 7 or a pharmaceutically acceptable salt thereof; and iv) a compound according to formula 8 or a pharmaceutically acceptable salt thereof.

In yet another embodiment according to the present invention, the one or more active components is:
a compound according to formula 1, or a pharmaceutically acceptable salt thereof; or a compound according to formula 3, or a pharmaceutically acceptable salt thereof; and
a compound according to formula 2, or a pharmaceutically acceptable salt thereof; or a compound according to formula 4, or a pharmaceutically acceptable salt thereof.

In another embodiment according to the present invention, the one or more active components is i) a compound according to formula 1, or a pharmaceutically acceptable salt thereof; and/or ii) a compound according to formula 3, or a pharmaceutically acceptable salt thereof.

In another embodiment according to the present invention, the one or more active components is i) a compound according to formula 2, or a pharmaceutically acceptable salt thereof; and/or ii) a compound according to formula 4, or a pharmaceutically acceptable salt thereof.

In another embodiment according to the present invention,
R₁ and R₂ are OH; and
the one or more active components is i) a compound according to formula 2, or a pharmaceutically acceptable salt thereof; and/or ii) a compound according to formula 4, or a pharmaceutically acceptable salt thereof.

In another embodiment according to the present invention,
R₁ and R₂ are O—CO—(CH₂)ₙ—CH₃;
n is 0, 1 or 2; preferably 0; and
the one or more active components is i) a compound according to formula 2, or a pharmaceutically acceptable salt thereof; and/or ii) a compound according to formula 4, or a pharmaceutically acceptable salt thereof.

In another embodiment according to the present invention,
R₁ and R₂ are OH; and
the one or more active components is i) a compound according to formula 5, or a pharmaceutically acceptable salt thereof; and/or ii) a compound according to formula 7, or a pharmaceutically acceptable salt thereof.

In another embodiment according to the present invention,
R₁ and R₂ are O—CO—(CH₂)ₙ—CH₃;
n is 0, 1 or 2; preferably 0; and
the one or more active components is i) a compound according to formula 5, or a pharmaceutically acceptable salt thereof; and/or ii) a compound according to formula 7, or a pharmaceutically acceptable salt thereof.

In another embodiment according to the present invention,
R₁ and R₂ are OH; and
the one or more active components is i) a compound according to formula 6, or a pharmaceutically acceptable salt thereof; and/or ii) a compound according to formula 8, or a pharmaceutically acceptable salt thereof.

In another embodiment according to the present invention,
R₁ and R₂ are O—CO—(CH₂)ₙ—CH₃;
n is 0, 1 or 2; preferably 0; and
the one or more active components is i) a compound according to formula 6, or a pharmaceutically acceptable salt thereof; and/or ii) a compound according to formula 8, or a pharmaceutically acceptable salt thereof.

In yet another embodiment according to the present invention, the one or more pharmaceutically acceptable excipients is selected from the group consisting of vegetable oils, triolein, soybean oil, safflower oil, sesame oil, castor oil, coconut oil, triglycerides, tributyrin, tricaproin, tricaprylin, vitamin E, antioxidants, α-tocopherol, ascorbic acid, deferoxamine mesylate, thioglycolic acid, emulsifiers, lecithin, polysorbate 80, methylcellulose, gelatin, serum albumin, sorbitan lauraute, sorbitan oleate, sorbitan trioleate, polyethylene glycol (PEG), PEG 400, polyethylene glycol-modified phosphatidylethanolamine (PEG-PE), poloxamers, glycerin, sorbitol, Xylitol, pH adjustment agents; sodium hydroxide, antimicrobial agents EDTA, sodium benzoate, benzyl alcohol and proteins such as albumin.

In a preferred embodiment, the one or more pharmaceutically acceptable excipients includes i) one or more components suitable to solubilize the one or more active components; and ii) one or more components with emulsifying properties. Furthermore, it is also preferred that the one or more pharmaceutically acceptable excipients includes one or more antioxidants, such as α-tocopherol, ascorbic acid, deferoxamine mesylate, thioglycolic acid. Further, it is also preferred that the one or more pharmaceutically acceptable excipients includes components for adjusting tonicity to physiological conditions, such as glycerin, sorbitol, Xylitol. Further, it is also preferred that the one or more pharmaceutically acceptable excipients includes PH adjusting agents, such as sodium hydroxide. Further, it is also preferred that the one or more pharmaceutically acceptable excipients includes one or more antimicrobial agents, such as EDTA, sodium benzoate, benzyl alcohol.

In a preferred embodiment, the one or more pharmaceutically acceptable excipients is phospholipid stabilized oil, such as phospholipid stabilized soybean oil and in particular the intralipid emulsion referred to in example 1.

Commercial products for providing injectable pharmaceutical compositions comprising lipids is known by the person skilled in the art and include products such as Miglyol 810, 812, Neobee M5, Captex 300, MONTANE™ 20 PPI, MONTANE™ 80 PPI, Pluronic F68, preformed emulsions (such as Lipofundin and Intralipid (Intralipid have been used in example 2) and others.

In yet another embodiment according to the present invention, the pharmaceutical composition further comprises at least one pharmaceutically acceptable carrier, in particular a lipid carrier such as liposomes and the like (including mixtures thereof).

In one embodiment according to the present invention, the composition has a pH that ranges from 5.5 to 8.5, such as a pH in the range 6-8, more preferably in the range 6.5-8 and most preferably it has a pH similar to the pH that normally prevails in the human body, such as a pH in the range 7.3 to 7.5.

In one embodiment according to the present invention R₁ is OH. In another embodiment according to the present invention R₂ is OH. In yet another embodiment according to the present invention, both R₁ and R₂ are OH.

In another embodiment according to the present invention, R₁ is OH and/or R₂ is OH. Preferably both R₁ and R₂ are OH.

In another embodiment according to the present invention, R₁ is O—CO—CH₃ and/or R₂ is O—CO—CH₃. Preferably both R₁ and R₂ are O—CO—CH₃.

In another embodiment according to the present invention, R₁ is O—CO—(CH₂)ₙ—CH₃ and/or R₂ is O—CO—(CH₂)ₙ—CH₃. Preferably both R₁ and R₂ are O—CO—(CH₂)ₙ—CH₃. n is 0, 1 or 2; more preferably n is 0, or 1; and most preferably n is 0.

In yet another embodiment according to the first aspect of the present invention, n is 0 or 1; most preferably n is 0.

In another embodiment according to the present invention, the one or more active components constitutes from 0.1-100% by dry-weight or by weight of the pharmaceutical composition, such as 0.5-100% by dry-weight or by weight of the pharmaceutical composition or 1-100% by dry-weight or by weight of the pharmaceutical composition.

In another embodiment according to the present invention, the one or more active components constitutes from 0.1-80% by dry-weight or by weight of the pharmaceutical composition, such as 0.5-60% by dry-weight or by weight of the pharmaceutical composition or 1-40% by dry-weight or by weight of the pharmaceutical composition.

In another embodiment according to the present invention, the one or more active components constitutes from 0.1-20% by dry-weight or by weight of the pharmaceutical composition, such as 0.5-10% by dry-weight or by weight of the pharmaceutical composition or 1-5% by dry-weight or by weight of the pharmaceutical composition.

In another embodiment according to the present invention, the one or more active components constitutes from 0.1-5% by dry-weight or by weight of the pharmaceutical composition, such as 0.5-5% by dry-weight or by weight of the pharmaceutical composition or 1-3% by dry-weight or by weight of the pharmaceutical composition.

In another embodiment according to the present invention, the one or more active components constitutes from 0.1-3% by dry-weight or by weight of the pharmaceutical composition, such as 0.5-3% by dry-weight or by weight of the pharmaceutical composition or 1-2% by dry-weight or by weight of the pharmaceutical composition.

In another embodiment according to the present invention, the one or more active components constitutes from 0.1-2% by dry-weight or by weight of the pharmaceutical composition, such as 0.1-1% by dry-weight or by weight of the pharmaceutical composition or 0.1-0.8% by dry-weight or by weight of the pharmaceutical composition.

In a preferred embodiment according to the present invention, the one or more active components constitutes from 10 to 99% by dry-weight or by weight of the pharmaceutical composition, such as from 15 to 99% by dry-weight or by weight of the pharmaceutical composition, from 20 to 99% by dry-weight or by weight of the pharmaceutical composition, from 25 to 99%, such as about 27%, by dry-weight or by weight of the pharmaceutical composition, from 35 to 99% by dry-weight or by weight of the pharmaceutical composition, from 55 to 99% by dry-weight or by weight of the pharmaceutical composition, from 75 to 99% by dry-weight or by weight of the pharmaceutical composition and most preferably from 80 to 99%, such as 85 to 95% (e.g. about 89%), by dry-weight or by weight of the pharmaceutical composition.

In yet another embodiment according to the present invention, molar ratio of lysoPC-DHA:lysoPC-EPA is in the range 1:1 to 10:1, such as in the range 1:1 to 7:1, or in the range 1:1 to 5:1, or in the range 1:1 to 3:1; or molar ratio of lysoPC-EPA:lysoPC-DHA is in the range 1:1 to 10:1, such as in the range 1:1 to 7:1, or in the range 1:1 to 5:1, or in the range 1:1 to 3:1; with the proviso that i) the number of moles of lysoPC-EPA is the number of moles 1-lysoPC-EPA+the number of moles 2-lysoPC-EPA; and ii) the number of moles of lysoPC-DHA is the number of moles 1-lysoPC-DHA+the number of moles 2-lysoPC-DHA.

In yet another embodiment according to the present invention,
R₁ and R₂ are OH; and
molar ratio of lysoPC-DHA:lysoPC-EPA is in the range 1:1 to 10:1, such as in the range 1:1 to 7:1, or in the range 1:1 to 5:1, or in the range 1:1 to 3:1; or molar ratio of lysoPC-EPA:lysoPC-DHA is in the range 1:1 to 10:1, such as in the range 1:1 to 7:1, or in the range 1:1 to 5:1, or in the range 1:1 to 3:1; with the proviso that i) the number of moles of lysoPC-EPA is the number of moles 1-lysoPC-EPA+the number of moles 2-lysoPC-EPA; and ii) the number of moles of lysoPC-DHA is the number of moles 1-lysoPC-DHA+the number of moles 2-lysoPC-DHA.

In yet another embodiment according to the present invention, molar ratio of 2-lysoPC-EPA/DHA:1-lysoPC-EPA/DHA is in the range 1:8 to 18:1, such as in the range 1:8 to 15:1 or in the range 1:8 to 10:1, with the proviso that i) the number of moles of 2-lysoPC-EPA/DHA is the number of moles 2-lysoPC-EPA+the number of moles 2-lysoPC-DHA; and ii) the number of moles of 1-lysoPC-EPA/DHA is the number of moles 1-lysoPC-EPA+the number of moles 1-lysoPC-DHA.

In yet another embodiment according to the present invention,
R₁ and R₂ are OH; and
molar ratio of 2-lysoPC-EPA/DHA:1-lysoPC-EPA/DHA is in the range 1:8 to 18:1, such as in the range 1:8 to 15:1 or in the range 1:8 to 10:1, with the proviso that i) the number of moles of 2-lysoPC-EPA/DHA is the number of moles 2-lysoPC-EPA+the number of moles 2-lysoPC-DHA; and ii) the number of moles of 1-lysoPC-EPA/DHA is the number of moles 1-lysoPC-EPA+the number of moles 1-lysoPC-DHA.

In one embodiment according to the present invention, the pharmaceutical composition contains less than 10% by dry-weight or weight of the pharmaceutical composition of a compound of general formula 9, wherein R₁ is OH and R₃ is O—CO—(CH₂)₁₂, such as less than 5% by dry-weight or weight of the pharmaceutical composition, less than 1% by dry-weight or weight of the pharmaceutical composition, less than 0.5% by dry-weight or weight of the pharmaceutical composition, less than 0.1% by dry-weight or weight of the pharmaceutical composition, less than 0.01% by dry-weight or weight of the pharmaceutical composition or less than 0.001% by dry-weight or weight of the pharmaceutical composition.

In one embodiment according to the present invention, the pharmaceutical composition contains less than 10% by dry-weight or weight of the pharmaceutical composition of a compound of general formula 9, wherein R₁ is OH and R₃ is O—CO—(CH₂)₁₄, such as less than 5% by dry-weight or weight of the pharmaceutical composition, less than 1% by dry-weight or weight of the pharmaceutical composition, less than 0.5% by dry-weight or weight of the pharmaceutical composition, less than 0.1% by dry-weight or weight of the pharmaceutical composition, less than 0.01% by dry-weight or weight of the pharmaceutical composition or less than 0.001% by dry-weight or weight of the pharmaceutical composition.

In one embodiment according to the present invention, the pharmaceutical composition contains less than 10% by dry-weight or weight of the pharmaceutical composition of a compound of general formula 9, wherein R₁ is OH and R₃ is O—CO—(CH₂)₁₆, such as less than 5% by dry-weight or weight of the pharmaceutical composition, less than 1% by dry-weight or weight of the pharmaceutical composition, less than 0.5% by dry-weight or weight of the pharmaceutical composition, less than 0.1% by dry-weight or weight of the pharmaceutical composition, less than 0.01% by dry-weight or weight of the pharmaceutical composition or less than 0.001% by dry-weight or weight of the pharmaceutical composition.

In one embodiment according to the present invention, the pharmaceutical composition contains less than 10% by dry-weight or weight of the pharmaceutical composition of a compound of general formula 10, wherein R₂ is OH and R₄ is O—CO—(CH₂)₁₂, such as less than 5% by dry-weight or weight of the pharmaceutical composition, less than 1% by dry-weight or weight of the pharmaceutical composition, less than 0.5% by dry-weight or weight of the pharmaceutical composition, less than 0.1% by dry-weight or weight of the pharmaceutical composition, less than 0.01% by dry-weight or weight of the pharmaceutical composition or less than 0.001% by dry-weight or weight of the pharmaceutical composition.

In one embodiment according to the present invention, the pharmaceutical composition contains less than 10% by dry-weight or weight of the pharmaceutical composition of a compound of general formula 10, wherein R₂ is OH and R₄ is O—CO—(CH₂)₁₄, such as less than 5% by dry-weight or weight of the pharmaceutical composition, less than 1% by dry-weight or weight of the pharmaceutical composition, less than 0.5% by dry-weight or weight of the pharmaceutical composition, less than 0.1% by dry-weight or weight of the pharmaceutical composition, less than 0.01% by dry-weight or weight of the pharmaceutical composition or less than 0.001% by dry-weight or weight of the pharmaceutical composition.

In one embodiment according to the present invention, the pharmaceutical composition contains less than 10% by dry-weight or weight of the pharmaceutical composition of a compound of general formula 10, wherein R₂ is OH and R₄ is O—CO—(CH₂)₁₆, such as less than 5% by dry-weight or weight of the pharmaceutical composition, less than 1% by dry-weight or weight of the pharmaceutical composition, less than 0.5% by dry-weight or weight of the pharmaceutical composition, less than 0.1% by dry-weight or weight of the pharmaceutical composition, less than 0.01% by dry-weight or weight of the pharmaceutical composition or less than 0.001% by dry-weight or weight of the pharmaceutical composition.

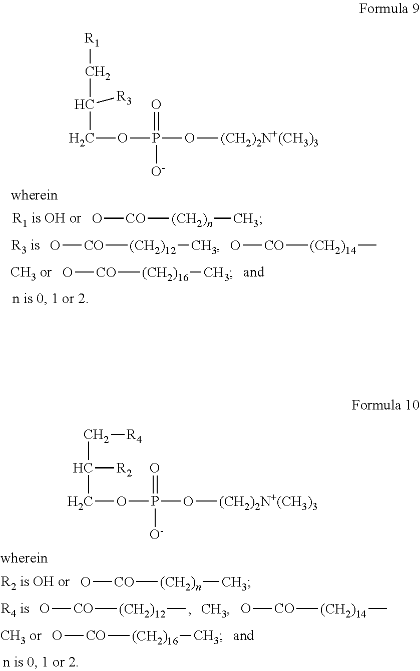

In one embodiment according to the present invention, the content of LPC molecules with a O—CO—(CH₂)₁₂—CH₃ moiety bound to the glycerol backbone of a LPC molecule is less than 10% of the LPC molecules of the pharmaceutical composition on a molar basis, such as less than 5% of the LPC molecules, less than 1% of the LPC molecules, less than 0.5% of the LPC molecules, less than 0.1% of the LPC molecules or less than 0.01% of the LPC molecules.

In one embodiment according to the present invention, the content of LPC molecules with a O—CO—(CH₂)₁₄—CH₃ moiety bound to the glycerol backbone of a LPC molecule is less than 10% of the LPC molecules of the pharmaceutical composition on a molar basis, such as less than 5% of the LPC molecules, less than 1% of the LPC molecules, less than 0.5% of the LPC molecules, less than 0.1% of the LPC molecules or less than 0.01% of the LPC molecules.

In one embodiment according to the present invention, the content of LPC molecules with a O—CO—(CH₂)₁₆—CH₃ moiety bound to the glycerol backbone of a LPC molecule is less than 10% of the LPC molecules of the pharmaceutical composition on a molar basis, such as less than 5% of the LPC molecules, less than 1% of the LPC molecules, less than 0.5% of the LPC molecules, less than 0.1% of the LPC molecules or less than 0.01% of the LPC molecules.

In one embodiment according to the present invention, the pharmaceutical composition further comprises phosphatidylcholine (PC). In one embodiment according to the present invention, at least one of the fatty acyl moieties of the PC molecule is an omega-3 fatty acyl; and preferably both of the fatty acyl moieties are omega-3 fatty acyls. The omega-3 fatty acyl preferably being selected from the group consisting of DHA, EPA, DPA and SDA.

In one embodiment, the phosphatidylcholine (PC) constitutes from 1-95% by dry-weight or by weight of the pharmaceutical composition, such as 5-80% by dry-weight or by weight of the pharmaceutical composition or 10-80% by dry-weight or by weight of the pharmaceutical composition. In another embodiment, the phosphatidylcholine (PC) constitutes from 10-70% by dry-weight or by weight of the pharmaceutical composition, such as 10-50% by dry-weight or by weight of the pharmaceutical composition or 5-50% by dry-weight or by weight of the pharmaceutical composition.

A PC molecule has a choline head group bound to one end of a glycerol backbone and the two other positions of the glycerol backbone are occupied by fatty acyl moieties. Thus, there are two fatty acyl moieties per PC molecule, i.e. two mol fatty acyl per mol PC.

A composition comprising 100 molecules of PC, wherein
8 of said PC molecules has two O—CO—(CH₂)₁₂—CH₃ moieties attached to the glycerol backbone;
4 of said PC molecules has one O—CO—(CH₂)₁₂—CH₃ moiety attached to the glycerol backbone; and
88 of said PC molecules has zero O—CO—(CH₂)₁₂—CH₃ moieties attached to the glycerol backbone;

will have a total of 200 fatty acyl groups of which 20 are a O—CO—(CH₂)₁₂—CH₃ moieties. In such a composition 10% of the fatty acyl moieties that are bound to the glycerol backbone of PC is a O—CO—(CH₂)₁₂—CH₃ moiety on a molar basis.

If the pharmaceutical composition of the present invention comprises PC, then it is preferred that less than 10% of the fatty acyl moieties that are bound to the glycerol backbone of PC is a O—CO—(CH₂)₁₂—CH₃ moiety, such as less than 5%, less than 1%, less than 0.5%, less than 0.1% or less than 0.01% on a molar basis.

If the pharmaceutical composition of the present invention comprises PC, then it is preferred that less than 10% of the fatty acyl moieties that are bound to the glycerol backbone of PC is a O—CO—(CH₂)₁₄—CH₃ moiety, such as less than 5%, less than 1%, less than 0.5%, less than 0.1% or less than 0.01% on a molar basis.

If the pharmaceutical composition of the present invention comprises PC, then it is preferred that less than 10% of the fatty acyl moieties that are bound to the glycerol backbone of PC is a O—CO—(CH₂)₁₆—CH₃ moiety, such as less than 5%, less than 1%, less than 0.5%, less than 0.1% or less than 0.01% on a molar basis.

In one embodiment according to the present invention, the pharmaceutical composition comprises PC. In a preferred embodiment, less than 10% of the fatty acyl moieties that are bound to the glycerol backbone of PC is a O—CO—(CH₂)₁₂—CH₃ moiety, such as less than 5%, less than 1%, less than 0.5%, less than 0.1% or less than 0.01% on a molar basis.

In one embodiment according to the present invention, the pharmaceutical composition comprises PC. In a preferred embodiment, less than 10% of the fatty acyl moieties that are bound to the glycerol backbone of PC is a O—CO—(CH₂)₁₄—CH₃ moiety, such as less than 5%, less than 1%, less than 0.5%, less than 0.1% or less than 0.01% on a molar basis.

In one embodiment according to the present invention, the pharmaceutical composition comprises PC. In a preferred embodiment, less than 10% of the fatty acyl moieties that are bound to the glycerol backbone of PC is a O—CO—(CH₂)₁₆—CH₃ moiety, such as less than 5%, less than 1%, less than 0.5%, less than 0.1% or less than 0.01% on a molar basis.

In one embodiment according to the present invention, the pharmaceutical composition does not contain any significant amounts of free omega-3 fatty acids, such as does not contain any free omega-3 fatty acids.

In another embodiment according to the present invention, the pharmaceutical composition contains less than 10% free omega-3 fatty acids by weight or dry-weight of the pharmaceutical composition, such as less than 5%, less than 1%, less than 0.5%, less than 0.1%, less than 0.01% or less than 0.001%.

In another embodiment according to the present invention, the pharmaceutical composition does not contain any significant amounts of free fatty acids, such as does not contain any free fatty acids.

In another embodiment according to the present invention, the pharmaceutical composition contains less than 10% free fatty acids by weight or dry-weight of the pharmaceutical composition, such as less than 5%, less than 1%, less than 0.5%, less than 0.1%, less than 0.01% or less than 0.001%.

In another embodiment according to the present invention, the pharmaceutical composition does not contain any significant amounts of free myristic acid, such as does not contain any free myristic acid.

In another embodiment according to the present invention, the pharmaceutical composition contains less than 10% free myristic acid by weight or dry-weight of the pharmaceutical composition, such as less than 5%, less than 1%, less than 0.5%, less than 0.1%, less than 0.01% or less than 0.001%.

In another embodiment according to the present invention, the pharmaceutical composition does not contain any significant amounts of free palmitic acid, such as does not contain any free palmitic acid.

In another embodiment according to the present invention, the pharmaceutical composition contains less than 10% free palmitic acid by weight or dry-weight of the pharmaceutical composition, such as less than 5%, less than 1%, less than 0.5%, less than 0.1%, less than 0.01% or less than 0.001%.

In some embodiments, the pharmaceutical composition of the present invention is provided for use in increasing the amount of EPA, DHA, DPA and/or SDA in a target tissue or organ, such as the brain, by intravascular administration, such as intravenous administration.

A second aspect the present invention relates to the pharmaceutical composition according to the first aspect of the present invention for use as a medicament, wherein the pharmaceutical composition is to be administered by intravascular administration, such as intravenous administration.

A third aspect the present invention relates to the pharmaceutical composition according to the first aspect of the present invention for use in prophylaxis and/or therapy, wherein the pharmaceutical composition is to be administered by intravascular administration, such as intravenous administration.

A fourth aspect the present invention relates to the pharmaceutical composition according to the first aspect of the present invention for use in prophylaxis and/or therapy of a condition which would benefit from increased levels of cerebral EPA levels, wherein the pharmaceutical composition is to be administered by intravascular administration, such as intravenous administration.

A fifth aspect the present invention relates to the pharmaceutical composition according to the first aspect of the present invention for use in prophylaxis and/or therapy of a condition which would benefit from increased levels of cerebral DHA levels, wherein the pharmaceutical composition is to be administered by intravascular administration, such as intravenous administration.

In one embodiment according to the fifth aspect of the present invention, the condition which would benefit from increased levels of cerebral DHA levels is a neurological condition.

In another embodiment according to the fifth aspect of the present invention, the neurological condition is depression, Schizophrenia, Alzheimer's disease, Parkinson's disease or traumatic brain injury.

In a preferred embodiment according to the fifth aspect of the present invention, the condition which would benefit from increased levels of cerebral DHA levels is traumatic brain injury.

In a preferred embodiment according to the fifth aspect of the present invention, the condition which would benefit from increased levels of cerebral DHA levels is traumatic brain injury and the pharmaceutical composition is administered in combination with i) progestogen or a prodrug thereof; and/or ii) estrogen or a prodrug thereof.

In a preferred embodiment according to the fifth aspect of the present invention, the condition which would benefit from increased levels of cerebral DHA levels is traumatic brain injury and the traumatic brain injury is from a closed head injury.

In one embodiment according to the fifth aspect of the present invention, the condition which would benefit from increased levels of cerebral DHA levels is post-traumatic stress disorder (PTSD) or anxiety.

A sixth aspect the present invention relates to the pharmaceutical composition according to the first aspect of the present invention for use in prophylaxis and/or therapy of a condition which would benefit from increased levels of cerebral DPA levels, wherein the pharmaceutical composition is to be administered by intravascular administration, such as intravenous administration.

A seventh aspect the present invention relates to the pharmaceutical composition according to the first aspect of the present invention for use in prophylaxis and/or therapy of a condition which would benefit from increased levels of cerebral SDA levels, wherein the pharmaceutical composition is to be administered by intravascular administration, such as intravenous administration.

It is to be understood that a condition which e.g. would benefit from increased levels of cerebral DHA levels may be treated by increasing the cerebral EPA levels since at least part of the EPA in the brain may be converted to DHA.

An eighth aspect of the present invention relates to the pharmaceutical composition according to the first aspect of the present invention, wherein R₁ and R₂ is OH, for use in prophylaxis and/or therapy; wherein the pharmaceutical composition is to be administered by intravascular administration, such as intravenous administration.

An ninth aspect of the present invention relates to the pharmaceutical composition according to the first aspect of the present invention, wherein R₁ and R₂ is OH, for use in prophylaxis and/or therapy of a condition which would benefit from increased cerebral DHA levels, wherein the pharmaceutical composition is to be administered by intravascular administration, such as intravenous administration.

In one embodiment according to the ninth aspect of the present invention, the condition which would benefit from increased cerebral DHA levels is a neurological condition, the neurological condition preferably being traumatic brain injury.

In one embodiment according to the ninth aspect of the present invention, the condition which would benefit from increased cerebral DHA levels is post-traumatic stress disorder (PTSD) or anxiety.

In one embodiment according to any one of aspects 2-9, the pharmaceutical composition is to be administered to a subject who is at risk of traumatic brain injury. The pharmaceutical composition is preferably administered in a prophylactically effective amount for a sufficient time period prior to engagement in an activity associated with a risk of traumatic brain injury to reduce the risk of pathological effects of traumatic brain injury. The traumatic head injury may be from a closed head injury.

A tenth aspect the present invention relates to the pharmaceutical composition according to the first aspect of the present invention for use to treat, prevent, or improve cognition and/or a cognitive disease, disorder or impairment (memory, concentration, learning (deficit)), or to treat or prevent neurodegenerative disorders; wherein the pharmaceutical composition is to be administered by intravascular administration, such as intravenous administration.

In some embodiments, the cognitive disease, disorder or impairment is selected from Attention Deficit Disorder (ADD), Attention Deficit Hyperactivity Disorder (ADHD), autism/autism spectrum disorder (ASD), (dyslexia, age-associated memory impairment and learning disorders, amnesia, mild cognitive impairment, cognitively impaired non-demented, pre-Alzheimer's disease, Alzheimer's disease, epilepsy, Pick's disease, Huntington's disease, Parkinson disease, Lou Gehrig's disease, pre-dementia syndrome, Lewy body dementia, dentatorubropallidoluysian atrophy, Freidreich's ataxia, multiple system atrophy, types 1, 2, 3, 6, 7 spinocerebellar ataxia, amyotrophic lateral sclerosis, familial spastic paraparesis, spinal muscular atrophy, spinal and bulbar muscular atrophy, age-related cognitive decline, cognitive deterioration, moderate mental impairment, mental deterioration as a result of ageing, conditions that influence the intensity of brain waves and/or brain glucose utilization, stress, anxiety, concentration and attention impairment, mood deterioration, general cognitive and mental well-being, neurodevelopmental, neurodegenerative disorders, hormonal disorders, neurological imbalance or any combinations thereof. In a specific embodiment, the cognitive disorder is memory impairment.

An eleventh aspect the present invention relates to the pharmaceutical composition according to the first aspect of the present invention for use to treat or prevent a cardiovascular disorder or metabolic syndrome; wherein the pharmaceutical composition is to be administered by intravascular administration, such as intravenous administration.

In some embodiments, the cardiovascular disorder is selected from atherosclerosis, arteriosclerosis, coronary heart (coronary artery) disease (CHD or CAD), acute coronary syndrome (or ACS), valvular heart disease, aortic and mitral valve disorders, arrhythmia/atrial fibrillation, cardiomyopathy and heart failure, angina pectoris, acute myocardial infarction (or AMI), hypertension, orthostatic hypotension, shock, embolism (pulmonary and venous), endocarditis, diseases of arteries, the aorta and its branches, disorders of the peripheral vascular system (peripheral arterial disease or PAD), Kawasaki disease, congenital heart disease (cardiovascular defects) and stroke (cerebrovascular disease), dyslipidemia, hypertriglyceridemia, hypertension, heart failure, cardiac arrhythmias, low HDL levels, high LDL levels, stable angina, coronary heart disease, acute myocardial infarction, secondary prevention of myocardial infarction, cardiomyopathy, endocarditis, type 2 diabetes, insulin resistance, impaired glucose tolerance, hypercholesterolemia, stroke, hyperlipidemia, hyperlipoproteinemia, chronic kidney disease, intermittent claudication, hyperphosphatemia, omega-3 deficiency, phospholipid deficiency, carotid atherosclerosis, peripheral arterial disease, diabetic nephropathy, hypercholesterolemia in HIV infection, acute coronary syndrome (ACS), non-alcoholic fatty liver disease/non-alcoholic steatohepatitis (NAFLD/NASH), arterial occlusive diseases, cerebral atherosclerosis, arteriosclerosis, cerebrovascular disorders, myocardial ischemia, coagulopathies leading to thrombus formation in a vessel and diabetic autonomic neuropathy.

A twelfth aspect the present invention relates to the pharmaceutical composition according to the first aspect of the present invention for use to inhibit, prevent, or treat inflammation or an inflammatory disease; wherein the pharmaceutical composition is to be administered by intravascular administration, such as intravenous administration.

In some embodiments, the inflammation or inflammatory disease is selected from organ transplant rejection; reoxygenation injury resulting from organ transplantation (see Grupp et al., J. Mol. Cell. Cardiol. 31: 297-303 (1999)) including, but not limited to, transplantation of the following organs: heart, lung, liver and kidney; chronic inflammatory diseases of the joints, including arthritis, rheumatoid arthritis, osteoarthritis and bone diseases associated with increased bone resorption; inflammatory bowel diseases (IBD) such as ileitis, ulcerative colitis (UC), Barrett's syndrome, and Crohn's disease (CD); inflammatory lung diseases such as asthma, acute respiratory distress syndrome (ARDS), and chronic obstructive pulmonary disease (COPD); inflammatory diseases of the eye including corneal dystrophy, trachoma, onchocerciasis, uveitis, sympathetic ophthalmitis and endophthalmitis; chronic inflammatory diseases of the gum, including gingivitis and periodontitis; inflammatory diseases of the kidney including uremic complications, glomerulonephritis and nephrosis; inflammatory diseases of the skin including sclerodermatitis, psoriasis and eczema; inflammatory diseases of the central nervous system, including chronic demyelinating diseases of the nervous system, multiple sclerosis, AIDS-related neurodegeneration and Alzheimer's disease, infectious meningitis, encephalomyelitis, Parkinson's disease, Huntington's disease, Epilepsy, amyotrophic lateral sclerosis and viral or autoimmune encephalitis, preeclampsia; chronic liver failure, brain and spinal cord trauma, and cancer. The inflammatory disease can also be a systemic inflammation of the body, exemplified by gram-positive or gram negative shock, hemorrhagic or anaphylactic shock, or shock induced by cancer chemotherapy in response to proinflammatory cytokines, e.g., shock associated with proinflammatory cytokines. Such shock can be induced, e.g., by a chemotherapeutic agent that is administered as a treatment for cancer. Other disorders include depression, obesity, allergic diseases, acute cardiovascular events, muscle wasting diseases, and cancer cachexia. Also, inflammation that results from surgery and trauma can be treated with the phospholipid compositions.

A thirteenth aspect the present invention relates to the pharmaceutical composition according to the first aspect of the present invention for use to treat a disease or condition associated with red blood cells and cell membranes, and in particular a disease or conditions associated with an abnormality in red blood cells of cell membranes; wherein the pharmaceutical composition is to be administered by intravascular administration, such as intravenous administration.

In some embodiments, the condition or disease is sickle cell disease, sickle cell anemia, or sickle cell trait. In some embodiments, the condition or disease is thalassemia (alpha-, beta- or delta-), thalassemia in combination with a hemoglobinopathy (Hemoglobin E, Hemoglobin S, or Hemoglobin C), splenomegaly, or membrane abnormities such as acanthocytes or spur/spike cells, codocytes (target cells), echinocytes (burr cells), elliptocytes and ovalocytes, spherocytes, stomatocytes (mouth cells) and degmacytes (“bite cells”).

In one embodiment according to any one of aspects 2-13, the pharmaceutical composition is to be administered to a subject of less than 10 years of age, such as less than 1 year of age, less than 1 month of age, or a newborn.

In one embodiment according to any one of aspects 2-13, the pharmaceutical composition is to be administered to a subject of more than 60 years of age, such as more than 70 year of age, more than 80 months of age, or to an elderly subject.

In one embodiment according to any one of aspects 2-13, the pharmaceutical composition is to be administered to a subject, wherein the subject is from about 10 to 20 years of age, from about 20 to 50 years of age from about 50 to 100 years of age, from about 60 to 100 years of age or from about 70 to 100 years of age.

In one embodiment according to any one of aspects 2-13, the pharmaceutical composition is to be administered to a subject, wherein the subject is female.

In one embodiment according to any one of aspects 2-13, the pharmaceutical composition is to be administered to a subject, wherein the subject is male.

In one embodiment according to the present invention, traumatic brain injury does not include brain injury induced by ischemia/reperfusion.

In some embodiments, the closed head injury is a concussion or contusion. A subject at risk for such injury can include, among others, a subject participating in an athletic event with occurrence of concussions. Exemplary subjects in this category include, among others, football players, boxers, and hockey players.

A fourteenth aspect of the present invention relates to the pharmaceutical composition according to the first aspect of the present invention for use in prophylaxis and/or therapy of a condition which would benefit from increased levels of EPA in the intestines, such as the intestinal mucosa, wherein the pharmaceutical composition is to be administered by intravascular administration, such as intravenous administration.

In one embodiment, the condition which would benefit from increased levels of EPA in the intestines, such as the intestinal mucosa, is selected from the group consisting of inflammatory bowel diseases (IBD), such as ileitis, ulcerative colitis (UC), Barrett's syndrome, and Crohn's disease (CD).

A fifteenth aspect of the present invention relates to the pharmaceutical composition according to the first aspect of the present invention for use in prophylaxis and/or therapy of a condition which would benefit from increased levels of DHA in the intestines, such as the intestinal mucosa, wherein the pharmaceutical composition is to be administered by intravascular administration, such as intravenous administration.

In one embodiment, the condition which would benefit from increased levels of DHA in the intestines, such as the intestinal mucosa, is selected from the group consisting of inflammatory bowel diseases (IBD), such as ileitis, ulcerative colitis (UC), Barrett's syndrome, and Crohn's disease (CD).

A sixteenth aspect of the present invention relates to the pharmaceutical composition according to the first aspect of the present invention for use in prophylaxis and/or therapy of a condition which would benefit from increased levels of EPA in the eye, such as the retina of the eye, wherein the pharmaceutical composition is to be administered by intravascular administration, such as intravenous administration.

In one embodiment, the condition which would benefit from increased levels of EPA in the eye, such as the retina of the eye, is selected from the group consisting of i) degenerative diseases of the retina, such as macular degeneration and in particular age-related macular degeneration (ARMD) and Retinitis Pigmentosa; ii) vascular Diseases of the Retina in Diabetics, such as proliferative retinopathy in diabetics, clinically significant macular edema in patients with diabetic retinopathy; iii) cataracts, such as age-related cataract, age-related cataracts in all patients, diabetics, and patients with ARMD.

A seventeenth aspect of the present invention relates to the pharmaceutical composition according to the first aspect of the present invention for use in prophylaxis and/or therapy of a condition which would benefit from increased levels of DHA in the eye, such as the retina of the eye, wherein the pharmaceutical composition is to be administered by intravascular administration, such as intravenous administration.

In one embodiment, the condition which would benefit from increased levels of DHA in the eye, such as the retina of the eye, is selected from the group consisting of i) degenerative diseases of the retina, such as macular degeneration and in particular age-related macular degeneration (ARMD) and Retinitis Pigmentosa; ii) vascular Diseases of the Retina in Diabetics, such as proliferative retinopathy in diabetics, clinically significant macular edema in patients with diabetic retinopathy; iii) cataracts, such as age-related cataract, age-related cataracts in all patients, diabetics, and patients with ARMD.

An second alternative aspect of the present invention relates to a pharmaceutical composition suitable for oral administration; the pharmaceutical composition comprising one or more active components; the one or more active components being selected from the group consisting of a compound according to any one of formula 1 to 8, or a pharmaceutically acceptable salt thereof, and any combination thereof

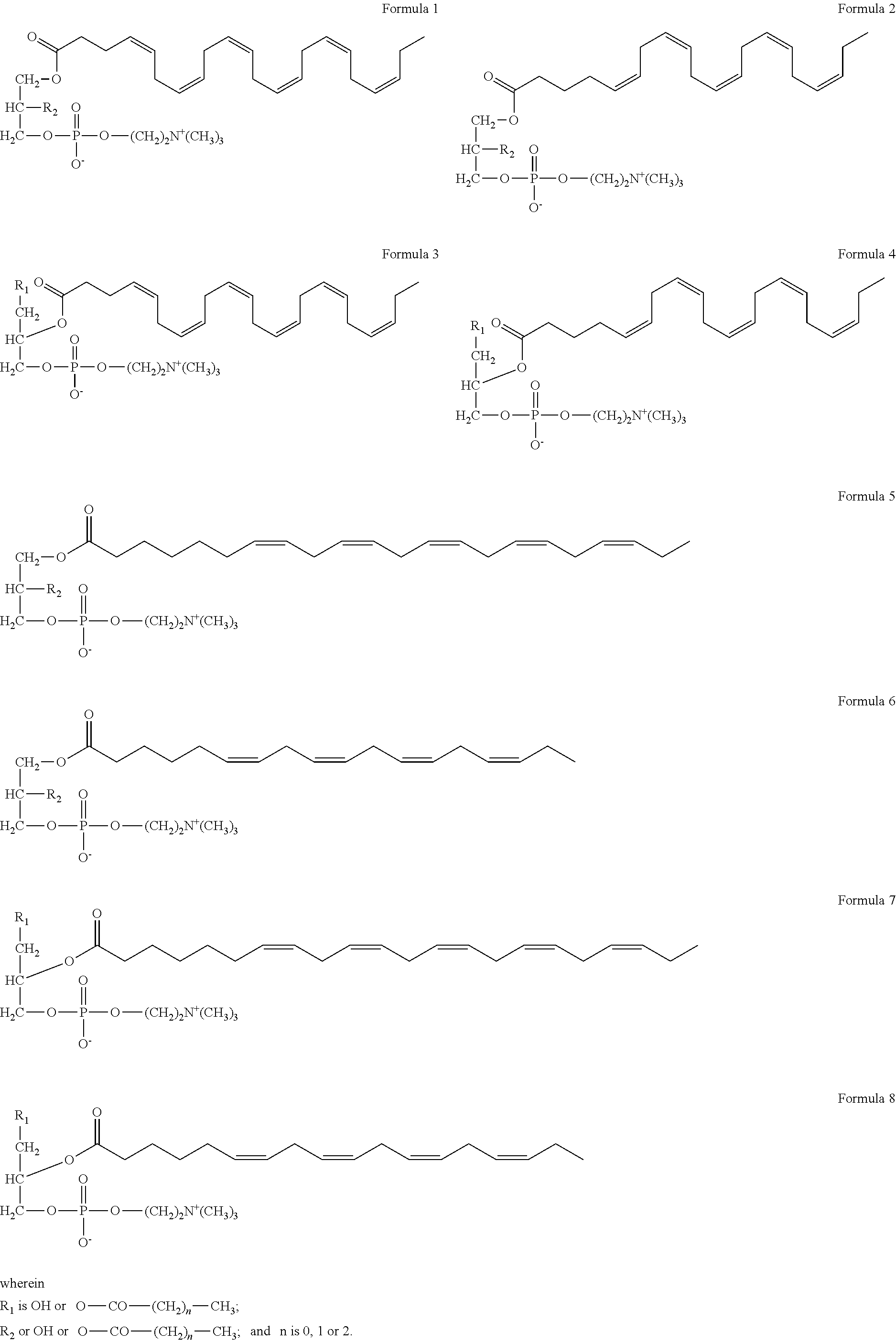

A preferred embodiment according to the second alternative aspect of the present invention relates to a pharmaceutical composition suitable for oral administration; the pharmaceutical composition comprising i) LPC-EPA, or a pharmaceutically acceptable salt thereof; and ii) LPC-DHA or a pharmaceutically acceptable salt thereof. Preferably the LPC-EPA and LPC-DHA constitutes from 10 to 99% by dry-weight or by weight of the pharmaceutical composition, such as from 15 to 99% by dry-weight or by weight of the pharmaceutical composition, from 20 to 99% by dry-weight or by weight of the pharmaceutical composition, from 25 to 99%, such as about 27%, by dry-weight or by weight of the pharmaceutical composition, from 35 to 99% by dry-weight or by weight of the pharmaceutical composition, from 55 to 99% by dry-weight or by weight of the pharmaceutical composition, from 75 to 99% by dry-weight or by weight of the pharmaceutical composition and most preferably from 80 to 99%, such as 85 to 95% (e.g. about 89%), by dry-weight or by weight of the pharmaceutical composition.

In one embodiment according to the second alternative aspect of the present invention, the one or more active components is a compound according to formula 1, wherein R₂ is OH or O—CO—(CH₂)ₙ—CH₃; and n is 0, 1 or 2.

In another embodiment according to the second alternative aspect of the present invention, the one or more active components is a compound according to formula 2, wherein R₂ is OH or O—CO—(CH₂)ₙ—CH₃; and n is 0, 1 or 2.

In another embodiment according to the second alternative aspect of the present invention, the one or more active components is a compound according to formula 3, wherein R₁ is OH or O—CO—(CH₂)ₙ—CH₃; and n is 0, 1 or 2.

In yet another embodiment according to the second alternative aspect of the present invention, the one or more active components is a compound according to formula 4, wherein R₁ is OH or O—CO—(CH₂)ₙ—CH₃; and n is 0, 1 or 2.

In yet another embodiment according to the second alternative aspect of the present invention, the one or more active components is a compound according to formula 5, wherein R₂ is OH or O—CO—(CH₂)ₙ—CH₃; and n is 0, 1 or 2.

In yet another embodiment according to the second alternative aspect of the present invention, the one or more active components is a compound according to formula 6, wherein R₂ is OH or O—CO—(CH₂)ₙ—CH₃; and n is 0, 1 or 2.

In yet another embodiment according to the second alternative aspect of the present invention, the one or more active components is a compound according to formula 7, wherein R₁ is OH or O—CO—(CH₂)ₙ—CH₃; and n is 0, 1 or 2.

In yet another embodiment according to the second alternative aspect of the present invention, the one or more active components is a compound according to formula 8, wherein R₁ is OH or O—CO—(CH₂)ₙ—CH₃; and n is 0, 1 or 2.

In yet another embodiment according to the second alternative aspect of the present invention, the one or more active components is a combination of two or more of the above mentioned active components.

In yet another embodiment according to the second alternative aspect of the present invention, the one or more active components is a combination of three, four, five or more of the above mentioned active components.

One embodiment according to the second alternative aspect of the present invention relates to a pharmaceutical composition according to the second alternative aspect of the present invention, with the proviso that: if the pharmaceutical composition comprises i) a compound according to formula 1, wherein R₂ is OH, or a pharmaceutically acceptable salt thereof; and/or ii) a compound according to formula 3, wherein R₁ is OH, or a pharmaceutically acceptable salt thereof; then the pharmaceutical composition further comprises at least one of the other active components referred to in the second alternative aspect of the present invention.

The expression “at least one of the other active components” recited above refers to at least one active component different from i) a compound according to formula 1, wherein R₂ is OH, or a pharmaceutically acceptable salt thereof; and different from ii) a compound according to formula 3, wherein R₁ is OH, or a pharmaceutically acceptable salt thereof.

In yet another embodiment according to the second alternative aspect, the one or more active components is i) a compound according to formula 1, or a pharmaceutically acceptable salt thereof; ii) a compound according to formula 2 or a pharmaceutically acceptable salt thereof; iii) a compound according to formula 3 or a pharmaceutically acceptable salt thereof; and iv) a compound according to formula 4 or a pharmaceutically acceptable salt thereof.

In yet another embodiment according to the second alternative aspect, the one or more active components is i) a compound according to formula 5, or a pharmaceutically acceptable salt thereof; ii) a compound according to formula 6 or a pharmaceutically acceptable salt thereof iii) a compound according to formula 7 or a pharmaceutically acceptable salt thereof; and iv) a compound according to formula 8 or a pharmaceutically acceptable salt thereof.

In yet another embodiment according to the second alternative aspect, the one or more active components is:
a compound according to formula 1, or a pharmaceutically acceptable salt thereof; or a compound according to formula 3, or a pharmaceutically acceptable salt thereof; and
a compound according to formula 2, or a pharmaceutically acceptable salt thereof; or a compound according to formula 4, or a pharmaceutically acceptable salt thereof.

In another embodiment according to the second alternative aspect, the one or more active components is i) a compound according to formula 1, or a pharmaceutically acceptable salt thereof; and/or ii) a compound according to formula 3, or a pharmaceutically acceptable salt thereof.

In another embodiment according to the second alternative aspect, the one or more active components is i) a compound according to formula 2, or a pharmaceutically acceptable salt thereof; and/or ii) a compound according to formula 4, or a pharmaceutically acceptable salt thereof.

In another embodiment according to the second alternative aspect,
R₁ and R₂ are OH; and
the one or more active components is i) a compound according to formula 2, or a pharmaceutically acceptable salt thereof; and/or ii) a compound according to formula 4, or a pharmaceutically acceptable salt thereof.

In another embodiment according to the second alternative aspect,
R₁ and R₂ are O—CO—(CH₂)ₙ—CH₃;
n is 0, 1 or 2; preferably 0; and
the one or more active components is i) a compound according to formula 2, or a pharmaceutically acceptable salt thereof; and/or ii) a compound according to formula 4, or a pharmaceutically acceptable salt thereof.

In another embodiment according to the second alternative aspect,
R₁ and R₂ are OH; and
the one or more active components is i) a compound according to formula 5, or a pharmaceutically acceptable salt thereof; and/or ii) a compound according to formula 7, or a pharmaceutically acceptable salt thereof.

In another embodiment according to the second alternative aspect,
R₁ and R₂ are O—CO—(CH₂)ₙ—CH₃;
n is 0, 1 or 2; preferably 0; and
the one or more active components is i) a compound according to formula 5, or a pharmaceutically acceptable salt thereof; and/or ii) a compound according to formula 7, or a pharmaceutically acceptable salt thereof.

In another embodiment according to the second alternative aspect,
R₁ and R₂ are OH; and
the one or more active components is i) a compound according to formula 6, or a pharmaceutically acceptable salt thereof; and/or ii) a compound according to formula 8, or a pharmaceutically acceptable salt thereof.

In another embodiment according to the second alternative aspect,
R₁ and R₂ are O—CO—(CH₂)ₙ—CH₃;
n is 0, 1 or 2; preferably 0; and
the one or more active components is i) a compound according to formula 6, or a pharmaceutically acceptable salt thereof; and/or ii) a compound according to formula 8, or a pharmaceutically acceptable salt thereof.

In one embodiment according to the second alternative aspect of the present invention R₁ is OH. In another embodiment according to the second alternative aspect of the present invention R₂ is OH. In yet another embodiment according to the second alternative aspect of the present invention, both R₁ and R₂ are OH.

In another embodiment according to the second alternative aspect of the present invention, R₁ is OH and/or R₂ is OH. Preferably both R₁ and R₂ are OH.

In another embodiment according to the second alternative aspect of the present invention, R₁ is O—CO—CH₃ and/or R₂ is O—CO—CH₃. Preferably both R₁ and R₂ are O—CO—CH₃.

In another embodiment according to the second alternative aspect of the present invention, R₁ is O—CO—(CH₂)ₙ—CH₃ and/or R₂ is O—CO—(CH₂)ₙ—CH₃. Preferably both R₁ and R₂ are O—CO—(CH₂)ₙ—CH₃. n is 0, 1 or 2; more preferably n is 0, or 1; and most preferably n is 0.

In yet another embodiment according to the second alternative aspect of the present invention, n is 0 or 1; most preferably n is 0.

In another embodiment according to the second alternative aspect of the present invention, the one or more active components constitutes from 0.1-100% by dry-weight or by weight of the pharmaceutical composition, such as 0.5-100% by dry-weight or by weight of the pharmaceutical composition or 1-100% by dry-weight or by weight of the pharmaceutical composition.

In another embodiment according to the second alternative aspect of the present invention, the one or more active components constitutes from 0.1-80% by dry-weight or by weight of the pharmaceutical composition, such as 0.5-60% by dry-weight or by weight of the pharmaceutical composition or 1-40% by dry-weight or by weight of the pharmaceutical composition.

In another embodiment according to the second alternative aspect of the present invention, the one or more active components constitutes from 0.1-20% by dry-weight or by weight of the pharmaceutical composition, such as 0.5-10% by dry-weight or by weight of the pharmaceutical composition or 1-5% by dry-weight or by weight of the pharmaceutical composition.

In another embodiment according to the second alternative aspect of the present invention, the one or more active components constitutes from 0.1-5% by dry-weight or by weight of the pharmaceutical composition, such as 0.5-5% by dry-weight or by weight of the pharmaceutical composition or 1-3% by dry-weight or by weight of the pharmaceutical composition.

In another embodiment according to the second alternative aspect of the present invention, the one or more active components constitutes from 0.1-3% by dry-weight or by weight of the pharmaceutical composition, such as 0.5-3% by dry-weight or by weight of the pharmaceutical composition or 1-2% by dry-weight or by weight of the pharmaceutical composition.

In another embodiment according to the second alternative aspect of the present invention, the one or more active components constitutes from 0.1-2% by dry-weight or by weight of the pharmaceutical composition, such as 0.1-1% by dry-weight or by weight of the pharmaceutical composition or 0.1-0.8% by dry-weight or by weight of the pharmaceutical composition.

In a preferred embodiment according to the second alternative aspect of the present invention, the one or more active components constitutes from 10 to 99% by dry-weight or by weight of the pharmaceutical composition, such as from 15 to 99% by dry-weight or by weight of the pharmaceutical composition, from 20 to 99% by dry-weight or by weight of the pharmaceutical composition, from 25 to 99%, such as about 27%, by dry-weight or by weight of the pharmaceutical composition, from 35 to 99% by dry-weight or by weight of the pharmaceutical composition, from 55 to 99% by dry-weight or by weight of the pharmaceutical composition, from 75 to 99% by dry-weight or by weight of the pharmaceutical composition and most preferably from 80 to 99%, such as 85 to 95% (e.g. about 89%), by dry-weight or by weight of the pharmaceutical composition.

In yet another embodiment according to the second alternative aspect of the present invention, molar ratio of lysoPC-DHA:lysoPC-EPA is in the range 1:1 to 10:1, such as in the range 1:1 to 7:1, or in the range 1:1 to 5:1, or in the range 1:1 to 3:1; or molar ratio of lysoPC-EPA:lysoPC-DHA is in the range 1:1 to 10:1, such as in the range 1:1 to 7:1, or in the range 1:1 to 5:1, or in the range 1:1 to 3:1; with the proviso that i) the number of moles of lysoPC-EPA is the number of moles 1-lysoPC-EPA+the number of moles 2-lysoPC-EPA; and ii) the number of moles of lysoPC-DHA is the number of moles 1-lysoPC-DHA+the number of moles 2-lysoPC-DHA.

In yet another embodiment according to the second alternative aspect of the present invention,
R₁ and R₂ are OH; and
molar ratio of lysoPC-DHA:lysoPC-EPA is in the range 1:1 to 10:1, such as in the range 1:1 to 7:1, or in the range 1:1 to 5:1, or in the range 1:1 to 3:1; or molar ratio of lysoPC-EPA:lysoPC-DHA is in the range 1:1 to 10:1, such as in the range 1:1 to 7:1, or in the range 1:1 to 5:1, or in the range 1:1 to 3:1; with the proviso that i) the number of moles of lysoPC-EPA is the number of moles 1-lysoPC-EPA+the number of moles 2-lysoPC-EPA; and ii) the number of moles of lysoPC-DHA is the number of moles 1-lysoPC-DHA+the number of moles 2-lysoPC-DHA.

In yet another embodiment according to the second alternative aspect of the present invention, molar ratio of 2-lysoPC-EPA/DHA:1-lysoPC-EPA/DHA is in the range 1:8 to 18:1, such as in the range 1:8 to 15:1 or in the range 1:8 to 10:1, with the proviso that i) the number of moles of 2-lysoPC-EPA/DHA is the number of moles 2-lysoPC-EPA+the number of moles 2-lysoPC-DHA; and ii) the number of moles of 1-lysoPC-EPA/DHA is the number of moles 1-lysoPC-EPA+the number of moles 1-lysoPC-DHA.

In yet another embodiment according to the second alternative aspect of the present invention,
R₁ and R₂ are OH; and
molar ratio of 2-lysoPC-EPA/DHA:1-lysoPC-EPA/DHA is in the range 1:8 to 18:1, such as in the range 1:8 to 15:1 or in the range 1:8 to 10:1, with the proviso that i) the number of moles of 2-lysoPC-EPA/DHA is the number of moles 2-lysoPC-EPA+the number of moles 2-lysoPC-DHA; and ii) the number of moles of 1-lysoPC-EPA/DHA is the number of moles 1-lysoPC-EPA+the number of moles 1-lysoPC-DHA.

In one embodiment according to the second alternative aspect of the present invention, the pharmaceutical composition contains less than 10% by dry-weight or weight of the pharmaceutical composition of a compound of general formula 9, wherein R₁ is OH and R₃ is O—CO—(CH₂)₁₂, such as less than 5% by dry-weight or weight of the pharmaceutical composition, less than 1% by dry-weight or weight of the pharmaceutical composition, less than 0.5% by dry-weight or weight of the pharmaceutical composition, less than 0.1% by dry-weight or weight of the pharmaceutical composition, less than 0.01% by dry-weight or weight of the pharmaceutical composition or less than 0.001% by dry-weight or weight of the pharmaceutical composition.

In one embodiment according to the second alternative aspect of the present invention, the pharmaceutical composition contains less than 10% by dry-weight or weight of the pharmaceutical composition of a compound of general formula 9, wherein R₁ is OH and R₃ is O—CO—(CH₂)₁₄, such as less than 5% by dry-weight or weight of the pharmaceutical composition, less than 1% by dry-weight or weight of the pharmaceutical composition, less than 0.5% by dry-weight or weight of the pharmaceutical composition, less than 0.1% by dry-weight or weight of the pharmaceutical composition, less than 0.01% by dry-weight or weight of the pharmaceutical composition or less than 0.001% by dry-weight or weight of the pharmaceutical composition.

In one embodiment according to the second alternative aspect of the present invention, the pharmaceutical composition contains less than 10% by dry-weight or weight of the pharmaceutical composition of a compound of general formula 9, wherein R₁ is OH and R₃ is O—CO—(CH₂)₁₆, such as less than 5% by dry-weight or weight of the pharmaceutical composition, less than 1% by dry-weight or weight of the pharmaceutical composition, less than 0.5% by dry-weight or weight of the pharmaceutical composition, less than 0.1% by dry-weight or weight of the pharmaceutical composition, less than 0.01% by dry-weight or weight of the pharmaceutical composition or less than 0.001% by dry-weight or weight of the pharmaceutical composition.

In one embodiment according to the second alternative aspect of the present invention, the pharmaceutical composition contains less than 10% by dry-weight or weight of the pharmaceutical composition of a compound of general formula 10, wherein R₂ is OH and R₄ is O—CO—(CH₂)₁₂, such as less than 5% by dry-weight or weight of the pharmaceutical composition, less than 1% by dry-weight or weight of the pharmaceutical composition, less than 0.5% by dry-weight or weight of the pharmaceutical composition, less than 0.1% by dry-weight or weight of the pharmaceutical composition, less than 0.01% by dry-weight or weight of the pharmaceutical composition or less than 0.001% by dry-weight or weight of the pharmaceutical composition.

In one embodiment according to the second alternative aspect of the present invention, the pharmaceutical composition contains less than 10% by dry-weight or weight of the pharmaceutical composition of a compound of general formula 10, wherein R₂ is OH and R₄ is O—CO—(CH₂)₁₄, such as less than 5% by dry-weight or weight of the pharmaceutical composition, less than 1% by dry-weight or weight of the pharmaceutical composition, less than 0.5% by dry-weight or weight of the pharmaceutical composition, less than 0.1% by dry-weight or weight of the pharmaceutical composition, less than 0.01% by dry-weight or weight of the pharmaceutical composition or less than 0.001% by dry-weight or weight of the pharmaceutical composition.

In one embodiment according to the second alternative aspect of the present invention, the pharmaceutical composition contains less than 10% by dry-weight or weight of the pharmaceutical composition of a compound of general formula 10, wherein R₂ is OH and R₄ is O—CO—(CH₂)₁₆, such as less than 5% by dry-weight or weight of the pharmaceutical composition, less than 1% by dry-weight or weight of the pharmaceutical composition, less than 0.5% by dry-weight or weight of the pharmaceutical composition, less than 0.1% by dry-weight or weight of the pharmaceutical composition, less than 0.01% by dry-weight or weight of the pharmaceutical composition or less than 0.001% by dry-weight or weight of the pharmaceutical composition.

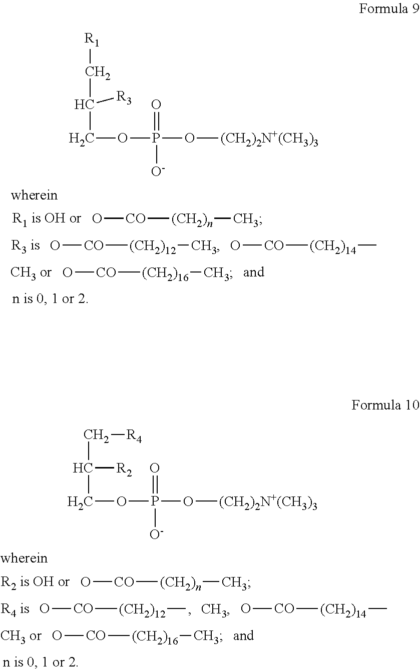

In one embodiment according to the second alternative aspect of the present invention, the content of LPC molecules with a O—CO—(CH₂)₁₂—CH₃ moiety bound to the glycerol backbone of a LPC molecule is less than 10% of the LPC molecules of the pharmaceutical composition on a molar basis, such as less than 5% of the LPC molecules, less than 1% of the LPC molecules, less than 0.5% of the LPC molecules, less than 0.1% of the LPC molecules or less than 0.01% of the LPC molecules.

In one embodiment according to the second alternative aspect of the present invention, the content of LPC molecules with a O—CO—(CH₂)₁₄—CH₃ moiety bound to the glycerol backbone of a LPC molecule is less than 10% of the LPC molecules of the pharmaceutical composition on a molar basis, such as less than 5% of the LPC molecules, less than 1% of the LPC molecules, less than 0.5% of the LPC molecules, less than 0.1% of the LPC molecules or less than 0.01% of the LPC molecules.

In one embodiment according to the second alternative aspect of the present invention, the content of LPC molecules with a O—CO—(CH₂)₁₆—CH₃ moiety bound to the glycerol backbone of a LPC molecule is less than 10% of the LPC molecules of the pharmaceutical composition on a molar basis, such as less than 5% of the LPC molecules, less than 1% of the LPC molecules, less than 0.5% of the LPC molecules, less than 0.1% of the LPC molecules or less than 0.01% of the LPC molecules.

In one embodiment according to the second alternative aspect of the present invention, the pharmaceutical composition further comprises phosphatidylcholine (PC). In one embodiment according to the second alternative aspect of the present invention, at least one of the fatty acyl moieties of the PC molecule is an omega-3 fatty acyl; and preferably both of the fatty acyl moieties are omega-3 fatty acyls. The omega-3 fatty acyl preferably being selected from the group consisting of DHA, EPA, DPA and SDA.

In one embodiment, the phosphatidylcholine (PC) constitutes from 1-95% by dry-weight or by weight of the pharmaceutical composition, such as 5-80% by dry-weight or by weight of the pharmaceutical composition or 10-80% by dry-weight or by weight of the pharmaceutical composition. In another embodiment, the phosphatidylcholine (PC) constitutes from 10-70% by dry-weight or by weight of the pharmaceutical composition, such as 10-50% by dry-weight or by weight of the pharmaceutical composition or 5-50% by dry-weight or by weight of the pharmaceutical composition.

A PC molecule has a choline head group bound to one end of a glycerol backbone and the two other positions of the glycerol backbone are occupied by fatty acyl moieties. Thus, there are two fatty acyl moieties per PC molecule, i.e. two mol fatty acyl per mol PC.

A composition comprising 100 molecules of PC, wherein
8 of said PC molecules has two O—CO—(CH₂)₁₂—CH₃ moieties attached to the glycerol backbone;
4 of said PC molecules has one O—CO—(CH₂)₁₂—CH₃ moiety attached to the glycerol backbone; and
88 of said PC molecules has zero O—CO—(CH₂)₁₂—CH₃ moieties attached to the glycerol backbone;

will have a total of 200 fatty acyl groups of which 20 are a O—CO—(CH₂)₁₂—CH₃ moieties. In such a composition 10% of the fatty acyl moieties that are bound to the glycerol backbone of PC is a O—CO—(CH₂)₁₂—CH₃ moiety on a molar basis.

If the pharmaceutical composition of the second alternative aspect of the present invention comprises PC, then it is preferred that less than 10% of the fatty acyl moieties that are bound to the glycerol backbone of PC is a O—CO—(CH₂)₁₂—CH₃ moiety, such as less than 5%, less than 1%, less than 0.5%, less than 0.1% or less than 0.01% on a molar basis.

If the pharmaceutical composition of the second alternative aspect of the present invention comprises PC, then it is preferred that less than 10% of the fatty acyl moieties that are bound to the glycerol backbone of PC is a O—CO—(CH₂)₁₄—CH₃ moiety, such as less than 5%, less than 1%, less than 0.5%, less than 0.1% or less than 0.01% on a molar basis.

If the pharmaceutical composition of the second alternative aspect of the present invention comprises PC, then it is preferred that less than 10% of the fatty acyl moieties that are bound to the glycerol backbone of PC is a O—CO—(CH₂)₁₆—CH₃ moiety, such as less than 5%, less than 1%, less than 0.5%, less than 0.1% or less than 0.01% on a molar basis.

In one embodiment according to the second alternative aspect of the present invention, the pharmaceutical composition comprises PC. In a preferred embodiment, less than 10% of the fatty acyl moieties that are bound to the glycerol backbone of PC is a O—CO—(CH₂)₁₂—CH₃ moiety, such as less than 5%, less than 1%, less than 0.5%, less than 0.1% or less than 0.01% on a molar basis.

In one embodiment according to the second alternative aspect of the present invention, the pharmaceutical composition comprises PC. In a preferred embodiment, less than 10% of the fatty acyl moieties that are bound to the glycerol backbone of PC is a O—CO—(CH₂)₁₄—CH₃ moiety, such as less than 5%, less than 1%, less than 0.5%, less than 0.1% or less than 0.01% on a molar basis.

In one embodiment according to the second alternative aspect of the present invention, the pharmaceutical composition comprises PC. In a preferred embodiment, less than 10% of the fatty acyl moieties that are bound to the glycerol backbone of PC is a O—CO—(CH₂)₁₆—CH₃ moiety, such as less than 5%, less than 1%, less than 0.5%, less than 0.1% or less than 0.01% on a molar basis.

In one embodiment according to the second alternative aspect of the present invention, the pharmaceutical composition does not contain any significant amounts of free omega-3 fatty acids, such as does not contain any free omega-3 fatty acids.

In another embodiment according to the second alternative aspect of the present invention, the pharmaceutical composition contains less than 10% free omega-3 fatty acids by weight or dry-weight of the pharmaceutical composition, such as less than 5%, less than 1%, less than 0.5%, less than 0.1%, less than 0.01% or less than 0.001%.

In another embodiment according to the second alternative aspect of the present invention, the pharmaceutical composition does not contain any significant amounts of free fatty acids, such as does not contain any free fatty acids.

In another embodiment according to the second alternative aspect of the present invention, the pharmaceutical composition contains less than 10% free fatty acids by weight or dry-weight of the pharmaceutical composition, such as less than 5%, less than 1%, less than 0.5%, less than 0.1%, less than 0.01% or less than 0.001%.

In another embodiment according to the second alternative aspect of the present invention, the pharmaceutical composition does not contain any significant amounts of free myristic acid, such as does not contain any free myristic acid.

In another embodiment according to the second alternative aspect of the present invention, the pharmaceutical composition contains less than 10% free myristic acid by weight or dry-weight of the pharmaceutical composition, such as less than 5%, less than 1%, less than 0.5%, less than 0.1%, less than 0.01% or less than 0.001%.

In another embodiment according to the second alternative aspect of the present invention, the pharmaceutical composition does not contain any significant amounts of free palmitic acid, such as does not contain any free palmitic acid.

In another embodiment according to the second alternative aspect of the present invention, the pharmaceutical composition contains less than 10% free palmitic acid by weight or dry-weight of the pharmaceutical composition, such as less than 5%, less than 1%, less than 0.5%, less than 0.1%, less than 0.01% or less than 0.001%.

In some embodiments, the pharmaceutical composition of the second alternative aspect of the present invention is provided for use in increasing the amount of EPA, DHA, DPA and/or SDA in a target tissue or organ, such as the brain, by intravascular administration, such as intravenous administration.

A further aspect of the present invention relates to the pharmaceutical composition according to the second alternative aspect of the present invention for use as a medicament, wherein the pharmaceutical composition is to be administered by oral administration.

A further aspect of the present invention relates to the pharmaceutical composition according to the second alternative aspect of the present invention for use in prophylaxis and/or therapy, wherein the pharmaceutical composition is to be administered by oral administration.

A further aspect of the present invention relates to the pharmaceutical composition according to the second alternative aspect of the present invention for use in prophylaxis and/or therapy of a condition which would benefit from increased levels of cerebral EPA and/or DHA levels, wherein the pharmaceutical composition is to be administered by oral administration.

In one embodiment the condition which would benefit from increased levels of cerebral DHA and/or EPA levels is a neurological condition such as depression, Schizophrenia, Alzheimer's disease, Parkinson's disease or traumatic brain injury.

In a preferred embodiment according to the fifth aspect of the present invention, the condition which would benefit from increased levels of cerebral DHA levels is traumatic brain injury.

In a preferred embodiment according to the fifth aspect of the present invention, the condition which would benefit from increased levels of cerebral DHA levels is traumatic brain injury and the pharmaceutical composition is administered in combination with i) progestogen or a prodrug thereof; and/or ii) estrogen or a prodrug thereof.

In a preferred embodiment according to the fifth aspect of the present invention, the condition which would benefit from increased levels of cerebral DHA levels is traumatic brain injury and the traumatic brain injury is from a closed head injury.

In one embodiment according to the fifth aspect of the present invention, the condition which would benefit from increased levels of cerebral DHA levels is post-traumatic stress disorder (PTSD) or anxiety.

In a another embodiment, the condition which would benefit from increased levels of cerebral DHA and/or EPA levels is traumatic brain injury, such as traumatic brain injury from a closed head injury.

In one embodiment, the condition which would benefit from increased levels of cerebral DHA and/or EPA levels is post-traumatic stress disorder (PTSD) or anxiety.

A further aspect of the present invention relates to the pharmaceutical composition according to the second alternative aspect of the present invention for use to treat, prevent, or improve cognition and/or a cognitive disease, disorder or impairment (memory, concentration, learning (deficit)), or to treat or prevent neurodegenerative disorders; wherein the pharmaceutical composition is to be administered by oral administration.

In some embodiments, the cognitive disease, disorder or impairment is selected from Attention Deficit Disorder (ADD), Attention Deficit Hyperactivity Disorder (ADHD), autism/autism spectrum disorder (ASD), (dyslexia, age-associated memory impairment and learning disorders, amnesia, mild cognitive impairment, cognitively impaired non-demented, pre-Alzheimer's disease, Alzheimer's disease, epilepsy, Pick's disease, Huntington's disease, Parkinson disease, Lou Gehrig's disease, pre-dementia syndrome, Lewy body dementia, dentatorubropallidoluysian atrophy, Freidreich's ataxia, multiple system atrophy, types 1, 2, 3, 6, 7 spinocerebellar ataxia, amyotrophic lateral sclerosis, familial spastic paraparesis, spinal muscular atrophy, spinal and bulbar muscular atrophy, age-related cognitive decline, cognitive deterioration, moderate mental impairment, mental deterioration as a result of ageing, conditions that influence the intensity of brain waves and/or brain glucose utilization, stress, anxiety, concentration and attention impairment, mood deterioration, general cognitive and mental well-being, neurodevelopmental, neurodegenerative disorders, hormonal disorders, neurological imbalance or any combinations thereof. In a specific embodiment, the cognitive disorder is memory impairment.

A further aspect of the present invention relates to the pharmaceutical composition according to the second alternative aspect of the present invention for use to treat or prevent a cardiovascular disorder or metabolic syndrome; wherein the pharmaceutical composition is to be administered by oral administration.

In some embodiments, the cardiovascular disorder is selected from atherosclerosis, arteriosclerosis, coronary heart (coronary artery) disease (CHD or CAD), acute coronary syndrome (or ACS), valvular heart disease, aortic and mitral valve disorders, arrhythmia/atrial fibrillation, cardiomyopathy and heart failure, angina pectoris, acute myocardial infarction (or AMI), hypertension, orthostatic hypotension, shock, embolism (pulmonary and venous), endocarditis, diseases of arteries, the aorta and its branches, disorders of the peripheral vascular system (peripheral arterial disease or PAD), Kawasaki disease, congenital heart disease (cardiovascular defects) and stroke (cerebrovascular disease), dyslipidemia, hypertriglyceridemia, hypertension, heart failure, cardiac arrhythmias, low HDL levels, high LDL levels, stable angina, coronary heart disease, acute myocardial infarction, secondary prevention of myocardial infarction, cardiomyopathy, endocarditis, type 2 diabetes, insulin resistance, impaired glucose tolerance, hypercholesterolemia, stroke, hyperlipidemia, hyperlipoproteinemia, chronic kidney disease, intermittent claudication, hyperphosphatemia, omega-3 deficiency, phospholipid deficiency, carotid atherosclerosis, peripheral arterial disease, diabetic nephropathy, hypercholesterolemia in HIV infection, acute coronary syndrome (ACS), non-alcoholic fatty liver disease/non-alcoholic steatohepatitis (NAFLD/NASH), arterial occlusive diseases, cerebral atherosclerosis, arteriosclerosis, cerebrovascular disorders, myocardial ischemia, coagulopathies leading to thrombus formation in a vessel and diabetic autonomic neuropathy.

A further aspect of the present invention relates to the pharmaceutical composition according to the second alternative aspect of the present invention for use to inhibit, prevent, or treat inflammation or an inflammatory disease; wherein the pharmaceutical composition is to be administered by oral administration.

In some embodiments, the inflammation or inflammatory disease is selected from organ transplant rejection; reoxygenation injury resulting from organ transplantation (see Grupp et al., J. Mol. Cell. Cardiol. 31: 297-303 (1999)) including, but not limited to, transplantation of the following organs: heart, lung, liver and kidney; chronic inflammatory diseases of the joints, including arthritis, rheumatoid arthritis, osteoarthritis and bone diseases associated with increased bone resorption; inflammatory bowel diseases (IBD) such as ileitis, ulcerative colitis (UC), Barrett's syndrome, and Crohn's disease (CD); inflammatory lung diseases such as asthma, acute respiratory distress syndrome (ARDS), and chronic obstructive pulmonary disease (COPD); inflammatory diseases of the eye including corneal dystrophy, trachoma, onchocerciasis, uveitis, sympathetic ophthalmitis and endophthalmitis; chronic inflammatory diseases of the gum, including gingivitis and periodontitis; inflammatory diseases of the kidney including uremic complications, glomerulonephritis and nephrosis; inflammatory diseases of the skin including sclerodermatitis, psoriasis and eczema; inflammatory diseases of the central nervous system, including chronic demyelinating diseases of the nervous system, multiple sclerosis, AIDS-related neurodegeneration and Alzheimer's disease, infectious meningitis, encephalomyelitis, Parkinson's disease, Huntington's disease, Epilepsy, amyotrophic lateral sclerosis and viral or autoimmune encephalitis, preeclampsia; chronic liver failure, brain and spinal cord trauma, and cancer. The inflammatory disease can also be a systemic inflammation of the body, exemplified by gram-positive or gram negative shock, hemorrhagic or anaphylactic shock, or shock induced by cancer chemotherapy in response to proinflammatory cytokines, e.g., shock associated with proinflammatory cytokines. Such shock can be induced, e.g., by a chemotherapeutic agent that is administered as a treatment for cancer. Other disorders include depression, obesity, allergic diseases, acute cardiovascular events, muscle wasting diseases, and cancer cachexia. Also, inflammation that results from surgery and trauma can be treated with the phospholipid compositions.

A further aspect of the present invention relates to the pharmaceutical composition according to the second alternative aspect of the present invention for use to treat a disease or condition associated with red blood cells and cell membranes, and in particular a disease or conditions associated with an abnormality in red blood cells of cell membranes; wherein the pharmaceutical composition is to be administered by oral administration.

In some embodiments, the condition or disease is sickle cell disease, sickle cell anemia, or sickle cell trait. In some embodiments, the condition or disease is thalassemia (alpha-, beta- or delta-), thalassemia in combination with a hemoglobinopathy (Hemoglobin E, Hemoglobin S, or Hemoglobin C), splenomegaly, or membrane abnormities such as acanthocytes or spur/spike cells, codocytes (target cells), echinocytes (burr cells), elliptocytes and ovalocytes, spherocytes, stomatocytes (mouth cells) and degmacytes (“bite cells”).

In one embodiment, the pharmaceutical composition is to be administered to a subject of less than 10 years of age, such as less than 1 year of age, less than 1 month of age, or a newborn.

In one embodiment, the pharmaceutical composition is to be administered to a subject of more than 60 years of age, such as more than 70 year of age, more than 80 months of age, or to an elderly subject.

In one embodiment, the pharmaceutical composition is to be administered to a subject, wherein the subject is from about 10 to 20 years of age, from about 20 to 50 years of age from about 50 to 100 years of age, from about 60 to 100 years of age or from about 70 to 100 years of age.

In one embodiment, the pharmaceutical composition is to be administered to a subject, wherein the subject is female.

In one embodiment, the pharmaceutical composition is to be administered to a subject, wherein the subject is male.

In one embodiment, traumatic brain injury does not include brain injury induced by ischemia/reperfusion.

In some embodiments, the closed head injury is a concussion or contusion. A subject at risk for such injury can include, among others, a subject participating in an athletic event with occurrence of concussions. Exemplary subjects in this category include, among others, football players, boxers, and hockey players.

A further aspect of the present invention relates to the pharmaceutical composition according to the second alternative aspect of the present invention for use in prophylaxis and/or therapy of a condition which would benefit from increased levels of EPA and/or DHA in the intestines, such as the intestinal mucosa, wherein the pharmaceutical composition is to be administered by oral administration.

In one embodiment, the condition which would benefit from increased levels of EPA and/or DHA in the intestines, such as the intestinal mucosa, is selected from the group consisting of inflammatory bowel diseases (IBD), such as ileitis, ulcerative colitis (UC), Barrett's syndrome, and Crohn's disease (CD).

A further aspect of the present invention relates to the pharmaceutical composition according to the second alternative aspect of the present invention for use in prophylaxis and/or therapy of a condition which would benefit from increased levels of EPA and/or DHA in the eye, such as the retina of the eye, wherein the pharmaceutical composition is to be administered by oral administration.

In one embodiment, the condition which would benefit from increased levels of EPA and/or DHA in the eye, such as the retina of the eye, is selected from the group consisting of i) degenerative diseases of the retina, such as macular degeneration and in particular age-related macular degeneration (ARMD) and Retinitis Pigmentosa; ii) vascular Diseases of the Retina in Diabetics, such as proliferative retinopathy in diabetics, clinically significant macular edema in patients with diabetic retinopathy; iii) cataracts, such as age-related cataract, age-related cataracts in all patients, diabetics, and patients with ARMD.

### BRIEF DESCRIPTION OF THE FIGURES

FIGS. 1-4 illustrates % of dosage per organ (brain, blood, kidneys and spleen respectively) following a single intravenous administration of [¹⁴C]-LPC-DHA to male albino rats at a target dose of 190 mg/kg. The experiments which forms basis for the data presented are disclosed in example 2.

FIGS. 5-8 illustrates % of dosage per organ (brain, blood, kidneys and spleen respectively) following a single intravenous administration of [¹⁴C]-LPC-EPA to male albino rats at a target dose of 190 mg/kg. The experiments which forms basis for the data presented are disclosed in example 2.

FIG. 9 illustrates general structure of the compartmental pharmacokinetic model employed in example 4 to study uptake in different issues of intravenously administered [¹⁴C]-LPC-DHA and [¹⁴C]-LPC-EPA.

FIG. 10a illustrates plasma and blood profiles of DHA following oral administration of [¹⁴C]-PL-DHA (where the phospholipid, PL, is a phosphatidylcholine, PC) according to the model disclosed in example 4. The line with the highest peak in the beginning represent the plasma profile.

FIG. 10b illustrates plasma and blood profiles of EPA following oral administration of [¹⁴C]-PL-EPA (where the phospholipid, PL, is a phosphatidylcholine, PC) according to the model disclosed in example 4. The line with the highest peak in the beginning represent the plasma profile.

FIG. 11a illustrates plasma and blood profiles of EPA following oral administration of [¹⁴C]-LPC-EPA according to the model disclosed in example 4. The two lines with peak concentration >1 μg/ml represent the plasma profile while the two other lines represent the blood profile.

FIG. 11b illustrates plasma and blood profiles of DHA following oral administration of [¹⁴C]-LPC-DHA according to the model disclosed in example 4. The two lines with peak concentration >1 μg/ml represent the plasma profile while the two other lines represent the blood profile.

FIG. 12a illustrates plasma and blood profiles of DHA following intravenous administration of [¹⁴C]-LPC-DHA according to the model disclosed in example 4.

FIG. 12b illustrates plasma and blood profiles of EPA following intravenous administration of [¹⁴C]-LPC-EPA according to the model disclosed in example 4.

FIG. 13 is a graphical representation of model simulated use of a long-term constant infusion (continuous infusion over 48 h of LPC-DHA) as compared to a bolus injection of DHA (as described in example 4).

FIG. 14 illustrates plasma LPC-DHA (ng/ml) taken after 0 weeks (T0, baseline), 2 weeks (T1) and 3 weeks (T2) of oral gavage according to the procedure described in Example 5.

FIG. 15 illustrates plasma LPC-EPA (ng/ml) taken after 0 weeks (T0, baseline), 2 weeks (T1) and 3 weeks (T2) of oral gavage according to the procedure described in Example 5.

FIG. 16 illustrates content of EPA in whole brain at different dosages of EPA according to the procedure of example 5. As can be seen there is a very strong dose-response relationship with higher doses of EPA being associated with higher brain EPA concentrations (ng/mg).

FIG. 17 illustrates brain DHA concentration in relation to total fatty acids for the six experimental groups recited in example 5.

FIG. 18 illustrates brain DHA concentration in relation to Arachidonic acid (ARA; 20:4 n-6) for the six experimental groups recited in example 5.

### DEFINITIONS

Throughout the present disclosure relevant terms are to be understood consistently with their typical meanings established in the relevant art, i.e. the art of pharmaceutical chemistry, medicine, biology, biochemistry and physiology. However, further clarifications and descriptions are provided for certain terms as set forth below.

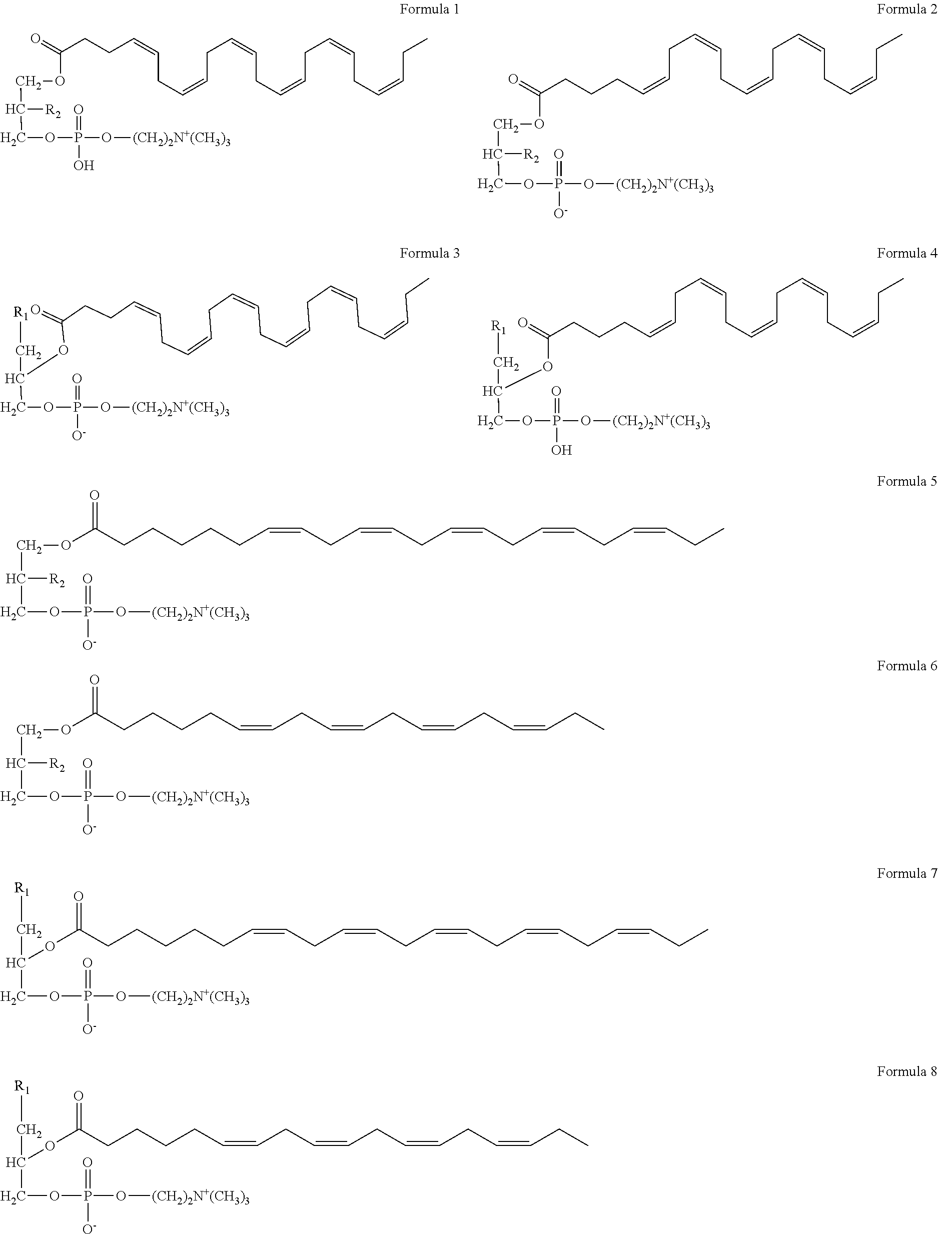

The terms “2-lysoPC-DHA” and “2-LPC-DHA” are used interchangeably herein and refer to a compound according to formula 1, wherein R₂ is OH.

The terms “2-lysoPC-EPA” and “2-LPC-EPA” are used interchangeably herein and refer to a compound according to formula 2, wherein R₂ is OH.

The terms “2-lysoPC-DPA” and “2-LPC-DPA” are used interchangeably herein and refer to a compound according to formula 5, wherein R₂ is OH.

The terms “2-lysoPC-SDA” and “2-LPC-SDA” are used interchangeably herein and refer to a compound according to formula 6, wherein R₂ is OH.

The terms “1-lysoPC-DHA” and “1-LPC-DHA” are used interchangeably herein and refer to a compound according to formula 3, wherein R₁ is OH.

The terms “1-lysoPC-EPA” and “1-LPC-EPA” are used interchangeably herein and refer to a compound according to formula 4, wherein R₁ is OH.

The terms “1-lysoPC-DPA” and “1-LPC-DPA” are used interchangeably herein and refer to a compound according to formula 7, wherein R₁ is OH.

The terms “1-lysoPC-SDA” and “1-LPC-SDA” are used interchangeably herein and refer to a compound according to formula 8, wherein R₁ is OH.

The terms “lysoPC-DHA” and “LPC-DHA” are used interchangeably herein and includes both 1-lysoPC-DHA and 2-lysoPC-DHA.

The terms “lysoPC-EPA” and “LPC-EPA” are used interchangeably herein and includes both 1-lysoPC-EPA and 2-lysoPC-EPA.

The terms “lysoPC-DPA” and “LPC-DPA” are used interchangeably herein and includes both 1-lysoPC-DPA and 2-lysoPC-DPA.

The terms “lysoPC-SDA” and “LPC-SDA” are used interchangeably herein and includes both 1-lysoPC-SDA and 2-lysoPC-SDA.

The term “EPA” refers to eicosapentaenoic acid.

The term “DHA” refers to docosahexaenoic acid.

The term “DPA” refers to n3-docosapentaenoic acid. The term “n3” specifying that the compound is an omega-3 fatty acid.

The term “SDA” refers to stearidonic acid.

The term “cerebral EPA levels” refers to the levels of EPA in the brain.

The term “cerebral DHA levels” refers to the levels of DHA in the brain.

The term “cerebral DPA levels” refers to the levels of DPA in the brain.

The term “cerebral SDA levels” refers to the levels of SDA in the brain.

The term “intravenous administration” as used herein refers to a mode of administration where a liquid substance is delivered directly into a vein. The intravenous route of administration can be used for injections (with a syringe at higher pressures) or infusions (typically using only the pressure supplied by gravity).

The term “pharmaceutically acceptable excipients” refer to substances different from the one or more active components referred to in the claims and which are commonly used with oily pharmaceuticals. Such excipients include, but are not limited to triolein, soybean oil, safflower oil, sesame oil, castor oil, coconut oil, triglycerides, tributyrin, tricaproin, tricaprylin, vitamin E, antioxidants, α-tocopherol, ascorbic acid, deferoxamine mesylate, thioglycolic acid, emulsifiers, lecithin, polysorbate 80, methylcellulose, gelatin, serum albumin, sorbitan lauraute, sorbitan oleate, sorbitan trioleate, polyethylene glycol (PEG), PEG 400, polyethylene glycol-modified phosphatidylethanolamine (PEG-PE), poloxamers, glycerin, sorbitol, Xylitol, pH adjustment agents; sodium hydroxide, antimicrobial agents EDTA, sodium benzoate, benzyl alcohol and proteins such as albumin. The pharmaceutically acceptable excipients must be acceptable in the sense of being compatible with the other ingredients of the composition and not deleterious to the recipient thereof.

Used herein, the term “pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of pharmaceutical salts properties, Selection, and Use; 2002.

The term “prophylaxis” means measures taken to prevent, rather than treat, diseases or conditions.

The term “prodrug” as used herein is a compound that, after administration, is metabolized (i.e., converted within the body) into a pharmacologically active drug.

As used herein, “traumatic brain injury” or “TBI” refers to acquired brain injury or a head injury when a trauma causes damage to the brain. The damage can be focal, i.e. confined to one area of the brain, or diffuse, involving more than one area of the brain.

As used herein, “closed head injury” refers to a brain injury when the head suddenly and violently hits an object, but the object does not break through the skull.

### DETAILED DESCRIPTION OF THE INVENTION

Unless specifically defined herein, all technical and scientific terms used have the same meaning as commonly understood by a skilled artisan in the fields of medicine, pharmacology, pharmaceutical chemistry, biology, biochemistry and physiology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail.

Where a numerical limit or range is stated herein, the endpoints are included. Also, all values and sub ranges within a numerical limit or range are specifically included as if explicitly written out.

As previously discussed, there are a number of medical conditions, including neurological conditions (such as TBI), PTSD and anxiety that are either associated with low cerebral omega-3 levels or which would benefit from increased levels of cerebral omega-3 levels. DHA, EPA, DPA and SDA are omega-3 fatty acids of particular interest in this respect.

Thus, there is a need for means to increase the levels of omega-3 fatty acids in the brain, and in particular to increase the levels of DHA, EPA, DPA and/or SDA in the brain.

Unlike other tissues, the uptake of omega-3 does not occur through the lipoprotein receptors in the brain and currently there is some discussion regarding the molecular carrier of omega-3 to the brain. Previous studies in animals have reported that DHA in the form of LPC passes through the BBB at a much faster rate than as free fatty acid. On the other hand, the recent kinetic studies of Chen et al. (Sci Rep. 2015; 5: 15791) suggested that free DHA in plasma is the major pool supplying the brain, although they also reported that the brain uptake of LPC-DHA was higher than that of free DHA.

Thus, there is a need for means to increase the levels of omega-3 fatty acids in serum, as this seems to be a prerequisite for increasing the levels of omega-3 fatty acids in the brain.

Since the form of omega-3 in serum may affect the uptake of these fatty acids into the brain, it may be of outermost importance to identify a carrier for the omega-3 which is able to deliver a form of omega-3 into serum which is efficiently taken up by the brain. Based on the recent identification of a specific transporter (Mfsd2a) in the endothelial cells of the BBB that selectively transports the LPC form of DHA across the BBB, there are reasons to assume that increased levels of LPC-omega-3 in serum would be an efficient way of increasing the content of the respective omega-3 fatty acid in the brain.

Thus, there is an urgent need for means to increase the levels of LPC-omega-3 in serum.

It has previously been suggested that dietary DHA provided in the sn-1 position of phosphatidylcholine (PC), or in the form of LPC in the diet, may be an effective way of increasing the levels of LPC-DHA in serum. However, in case of a neurological condition, such as TBI, the time from intake of dietary DHA until a raise in the levels of LPC-DHA in serum may be of outermost importance.

Thus, there is an urgent need in the art for means to increase the levels of LPC-DHA in serum at a fast rate.

Furthermore, LPC is found only in trace amounts in most animal tissues since greater concentrations are known to facilitate disruption of cell membranes (US2016/0022711, PharmSciTech, Vol. 11, No. 4, December 2010).

Thus, there is a need in the art for means to increase the levels of LPC-omega-3 in serum, in particular to increase the levels of LPC-DHA, LPC-EPA, LPC-DPA and/or LPC-SDA in serum, without causing unacceptable degree of cell membrane disruption and other potential side effects.

Another issue that should be considered is the need of a continuous supply of DHA into the brain. It is well known that administered drugs typically are removed from the circulation by various elimination processes, and such processes for elimination of LPC-omega-3 may of course affect the concentration of LPC-omega-3 in serum over time which also would be assumed to have a direct negative effect on the uptake into brain.

Thus, there is an urgent need in the art for means to keep high levels of LPC-omega-3 in serum over time as this is assumed to be a prerequisite for ensuring a continuous supply of omega-3 into the brain.

In the search for a solution to the above-mentioned needs, a significant amount of resources was invested focusing on oral uptake of various forms of omega-3 fatty acids, including studies on oral uptake of LPC-omega-3; and in particular LPC-DHA and LPC-EPA (PCT/IB2018/0001588).

Even though the results of that project (oral uptake of LPC-DHA and LPC-EPA) were impressive with respect of the uptake of the omega-3 fatty acids into the brain, there was a continuous discussion on how the uptake into the brain could be improved even further. Alternative forms of omega-3 fatty acids, how the fatty acids were to be formulated and also different encapsulation techniques were thoroughly discussed. It was also discussed whether it would be of interest to investigate alternative ways of administering the omega-3 fatty acids.

Parenteral administration, and in particular intravascular administration such as intravenous administration, of omega-3 fatty acids may provide an increased level of omega-3 fatty acids in serum at a fast rate, which may result in an increased level of omega-3 fatty acids into the brain at a fast rate. Furthermore, this may assumingly also be an effective way of by-passing the negative effects of the enzymes of the digestive system experienced by the oral route. However, it was also acknowledged that it may be a true risk that the fast increase in the levels of LPC-omega-3 in serum may cause unacceptable degree of cell membrane disruption and there may also be other potential side effects. Furthermore, since LPC is found only in trace amounts in most animal tissues, and high amounts in serum are known to be associated with side effects (i.a. disruption of cell membranes), there is also a high risk that there are effective mechanisms for eliminating such compounds from the circulation which would be assumed to have a negative effect on uptake of omega-3 into the brain over time. Further, there is always a question of patient compliance when going from oral route to a parenteral route; so, the effect of parenteral route should be significantly better than the oral route if it is to be of any commercial interest.

Despite the above-mentioned risks, it was decided to investigate further whether intravascular administration, in particular intravenous administration, of omega-3 fatty acids, in particular LPC-EPA and LPC-DHA, would represent a promising strategy to increase the levels of omega-3 fatty acids in the brain without causing unacceptable side effects.

Since it was already known that LPC is an efficient carrier for transporting molecules across the BBB, it was decided to use LPC-omega-3 in this study. In order to be able to measure the amount of omega-3 fatty acid that has been transported into the brain, it was decided to use LPC-omega-3, where the omega-3 fatty acid was labelled with a radioactive marker. Further, in order to ensure that it is only non-oxidized forms of the fatty acids that are being measured, it was decided to put the radioactive marker on the acyl-carbon of the fatty acid moiety, i.e. carbon no. 1 (example 1 provides an illustration indicating where the radioactive marker is located).

The Mfsd2a transporter at the BBB is known to specifically transport LPC-omega-3 but not free omega-3. It has been previously suggested that the transport across the BBB is not specific with respect to the fatty acid bound to the LPC molecule, but there is evidence indicating that the fatty acid bound to LPC needs to be of a certain length in order to be transported across the BBB. A length of 14 carbon atoms or more have been indicted in the prior art to be essential for transport across the BBB by the Mfsd2a transporter. DHA, EPA, SDA and DPA are considered to be of high importance with respect to positive health effects in humans, and all of these have more than 14 carbon atoms. Thus, based on the information we have at date, each and all of these fatty acids should be transported efficiently across the BBB when bound to LPC. LPC-DHA and LPC-EPA were therefore selected as model molecules in the present study, but all data provided herein regarding uptake into the brain are also believed to indicate expected uptake profiles of the other two omega-3 fatty acids referred to above, i.e. SDA and DPA.

Since LPC-DHA and LPC-EPA were to be administered by intravenous administration in this study, it was decided to mix the active components with one or more pharmaceutically acceptable excipients. Intralipid (IV) provided by Sigma Aldrich is compatible with oily substances and was therefore selected as the one or more pharmaceutically acceptable excipients. Reference is made to example 1 for further details to the pharmaceutical composition that was used in this study.

16 male Sprague Dawley rats received a single intravenous administration of either LPC-DHA or LPC-EPA. The dose was administered directly into a tail vein as a slow bolus over 30 seconds. A single rat was euthanized by overdose of carbon dioxide gas at each of the following times: 0.5, 3, 8, 24, 72, 96, 168 and 336 hours post-dose. Each carcass was snap frozen in a hexane/solid carbon dioxide mixture immediately after collection and were then stored at approximately −20° C., pending further analysis.

The frozen carcasses were subjected to quantitative whole-body autoradiography, as detailed in example 2, to study the uptake of DHA and EPA into the brain at 0.5, 3, 8, 24, 72, 96, 168 and 336 hours post-dose.

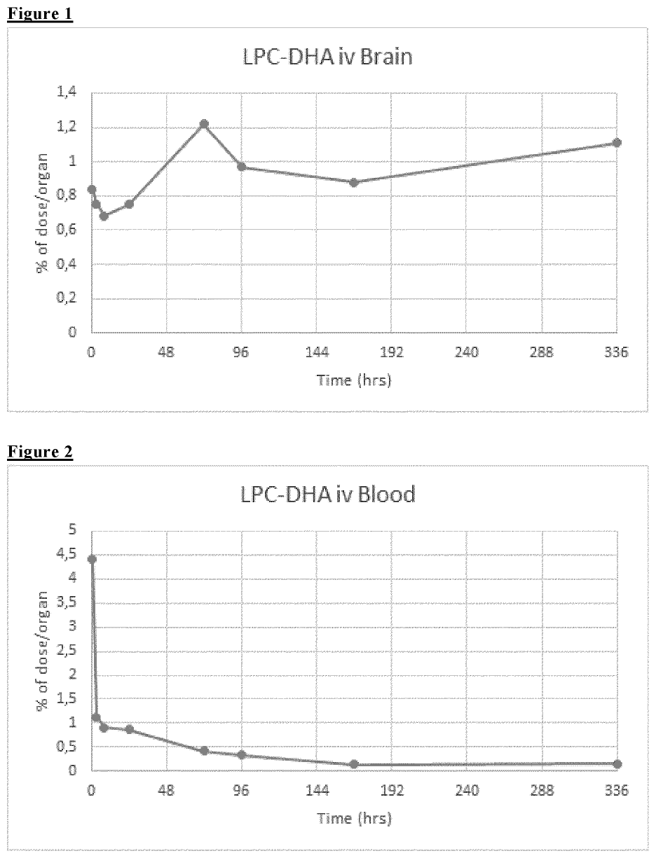

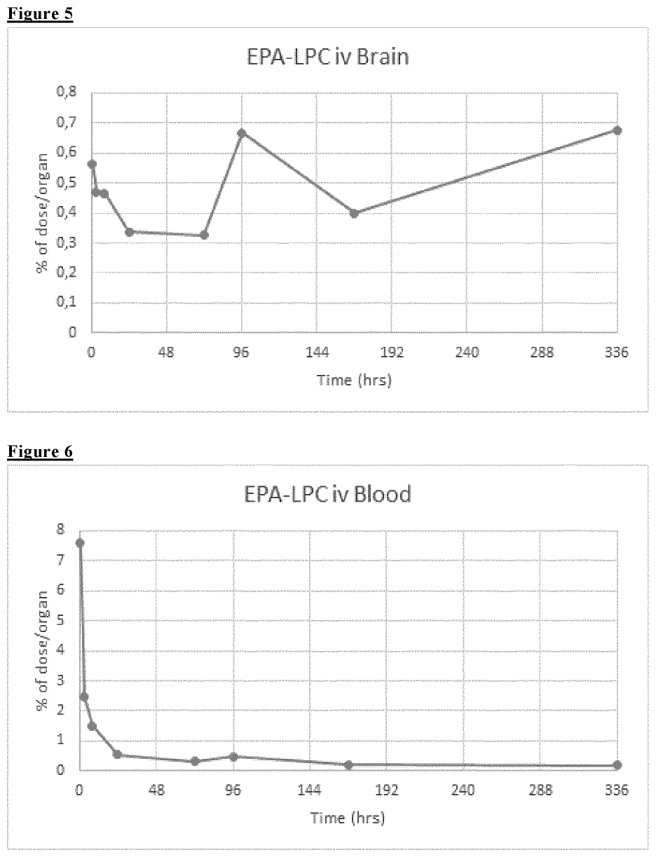

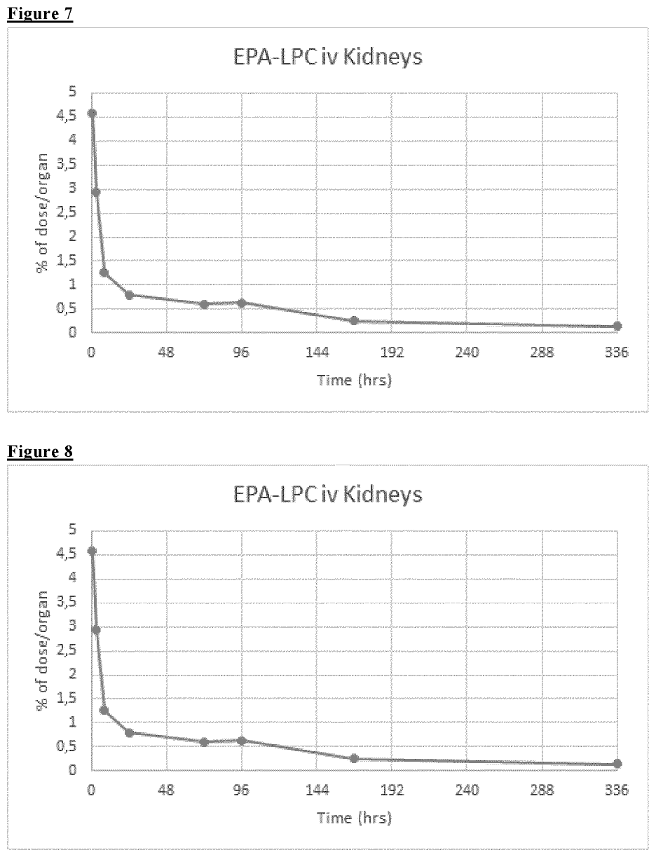

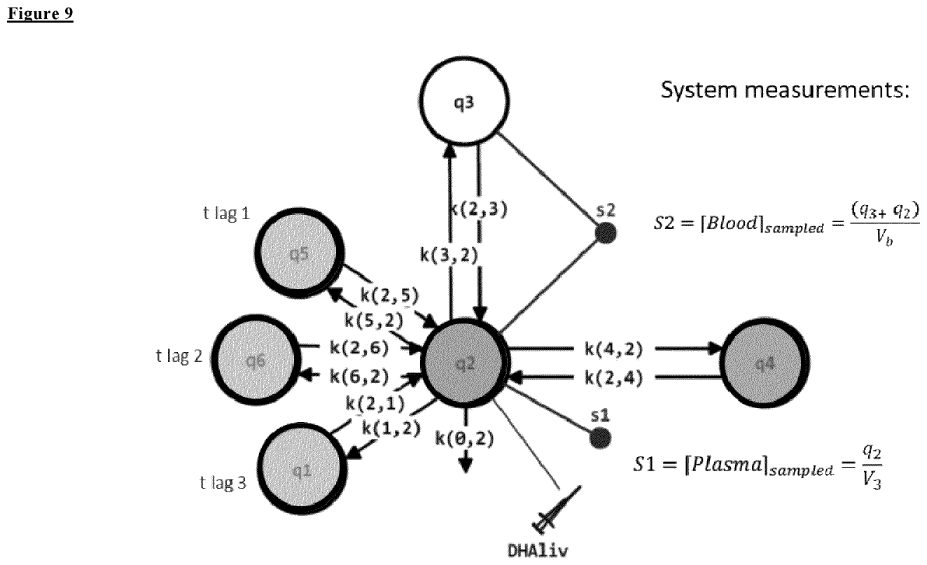

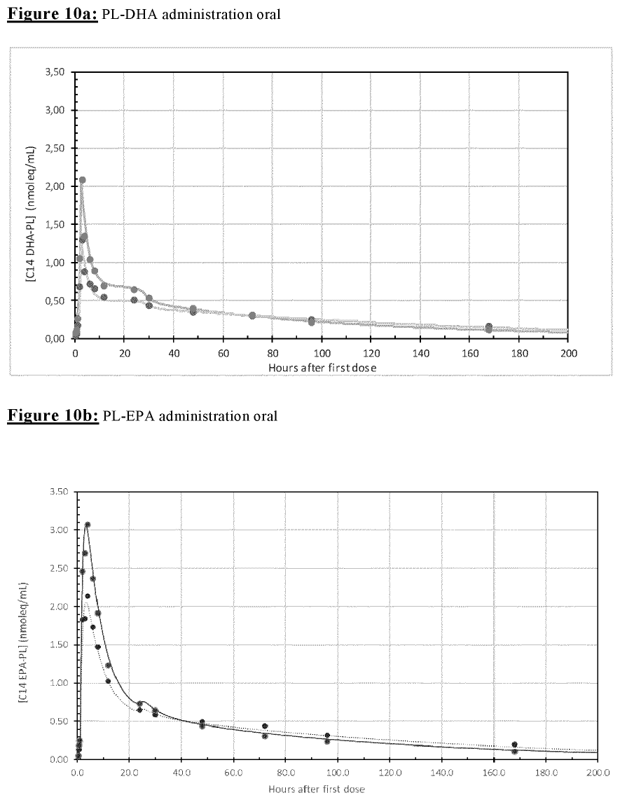

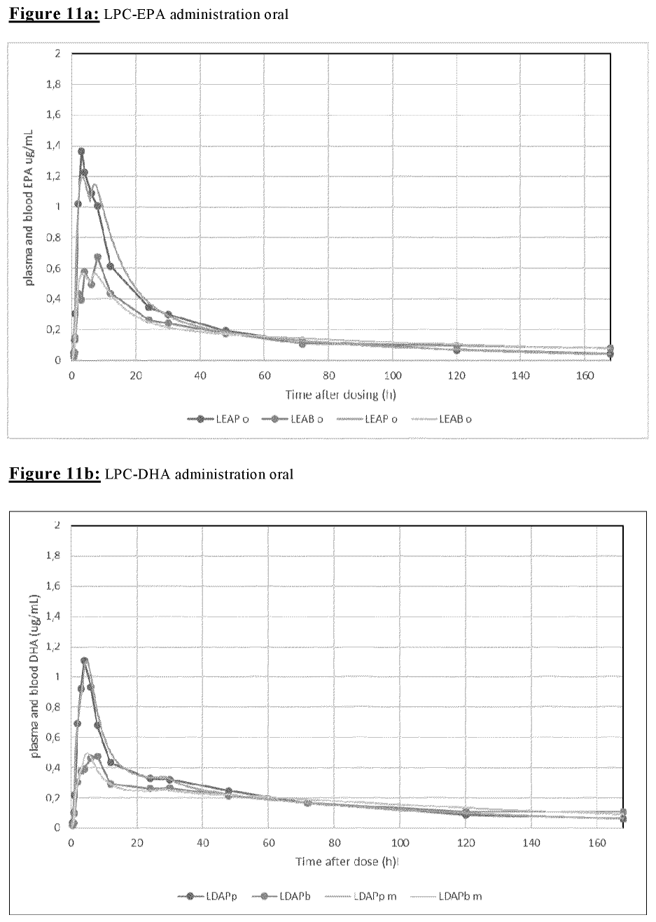

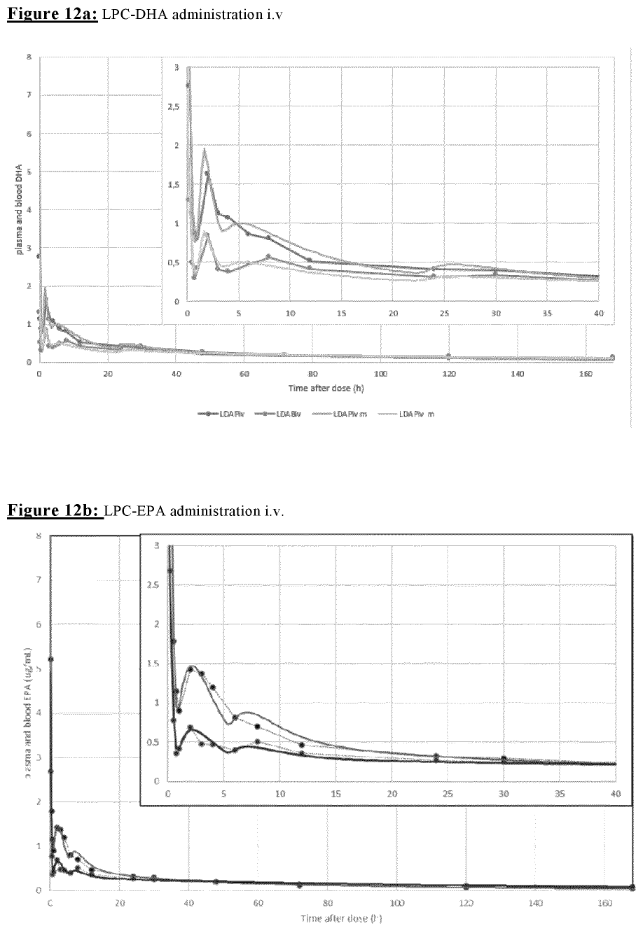

The final results of LPC-DHA are presented in example 2, table 1.1 and the data are also illustrated in FIG. 1-4. The final results of LPC-EPA are presented in example 2, table 2.1 and the data are also illustrated in FIGS. 5-8.

The first result that was received was the data related to the level of LPC-DHA in blood (FIG. 2). As expected, intravenous administration of LPC-DHA resulted in an immediate and high increase in the level of LPC-DHA in blood. However, the level of LPC-DHA also declined very quickly with time, clearly indicating that there are effective mechanisms for eliminating that compound from the blood. Since the level of LPC-DHA in blood is likely to be of high importance with respect to uptake in brain, it was acknowledged that it may be an issue with elimination that needs to be solved in order to ensure continues and high uptake of DHA into the brain.

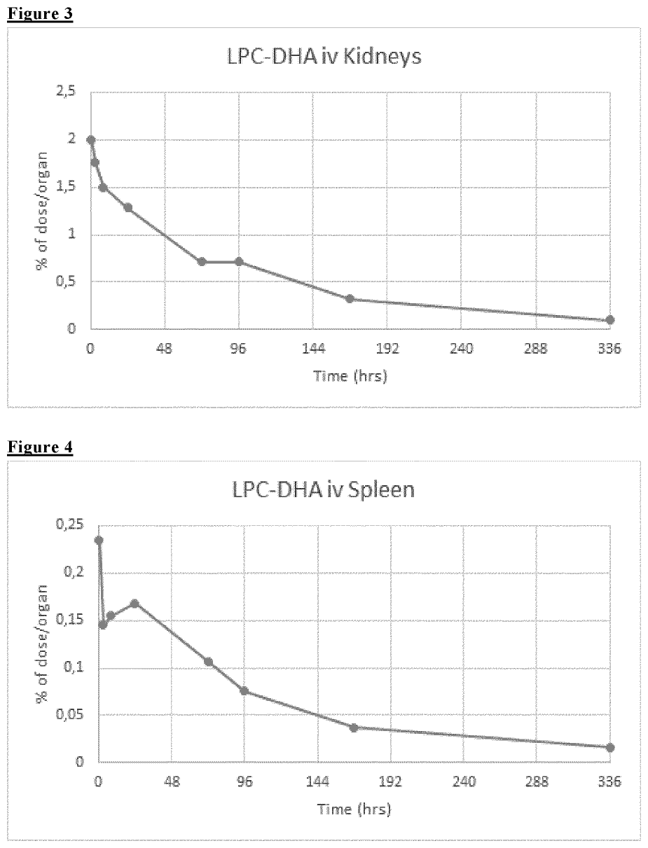

The next result that was received was the data related to uptake of LPC-DHA in kidney (FIG. 3). As expected, the amount of LPC-DHA in the kidneys over time followed the trend seen for blood. There was an immediate and high increase in the level of LPC-DHA in the kidneys at the time of dosing, but the level of LPC-DHA declined very quickly with time. Similar results were also seen for LPC-DHA uptake into the spleen (FIG. 4).

Based on the above results, it was expected that the amount of LPC-DHA in the brain would be high shortly after dosing but also that the amount of LPC-DHA in the brain would decline quickly with time; similar to what have been seen for blood, spleen and the kidneys. However, in contrast to what was expected; the results from the uptake studies of the brain (FIG. 1) surprisingly demonstrated that the amount of LPC-DHA in the brain did not follow the trend seen for blood, kidney and spleen. In contrast, intravenous administration of LPC-DHA resulted in an immediate and high increase in the level of LPC-DHA in the brain, and the level of LPC-DHA continued to increase with time far beyond the point where the level of LPC-DHA in blood declined significantly. These highly surprising results clearly demonstrating that intravenous administration of LPC-DHA may be a very effective way of increasing DHA levels in brain at a fast rate and also for keeping high levels of DHA in brain for a prolonged period of time, even after only one injection.

The results related to LPC-DHA (FIG. 1-4) are similar to the results obtained for LPC-EPA (FIG. 5-8), clearly indicating that intravenous administration of LPC-omega-3 may be a very effective way of increasing omega-3 levels in brain at a fast rate and also to keep the omega-3 levels in brain for a prolonged period of time; even after only one injection. Even though similar data is likely to be obtained for other omega-3 fatty acids, DHA, EPA, DPA, SDA and maybe also ALA are considered to be of particular interest in the present application.

The data presented herein in respect of LPC-EPA are based on the measured amount of radioactivity present in the brain after intravenous administration of radiolabeled LPC-EPA. Thus, it is to be understood that the data presented herein does not necessarily reflect the fate of the EPA molecule per se. If e.g. EPA is transformed into DHA within the brain, the data presented herein likely represents the amount of radiolabeled EPA+radiolabeled DHA. Similar may also apply to the data presented in respect of LPC-DHA.

In view of the examples presented herein, it is asserted that all of the above listed needs in the art have been solved by the pharmaceutical composition of the claimed invention, and in particular the pharmaceutical composition of the claimed invention for use as a medicament wherein the medicament is administered by intravascular administration and in particular intravenous administration.

Thus, a first aspect the present invention relates to a pharmaceutical composition suitable intravascular administration, such as intravenous administration; the pharmaceutical composition comprising one or more active components and one or more pharmaceutically acceptable excipients; the one or more active components being selected from the group consisting of a compound according to any one of formula 1 to 8, or a pharmaceutically acceptable salt thereof, and any combination thereof

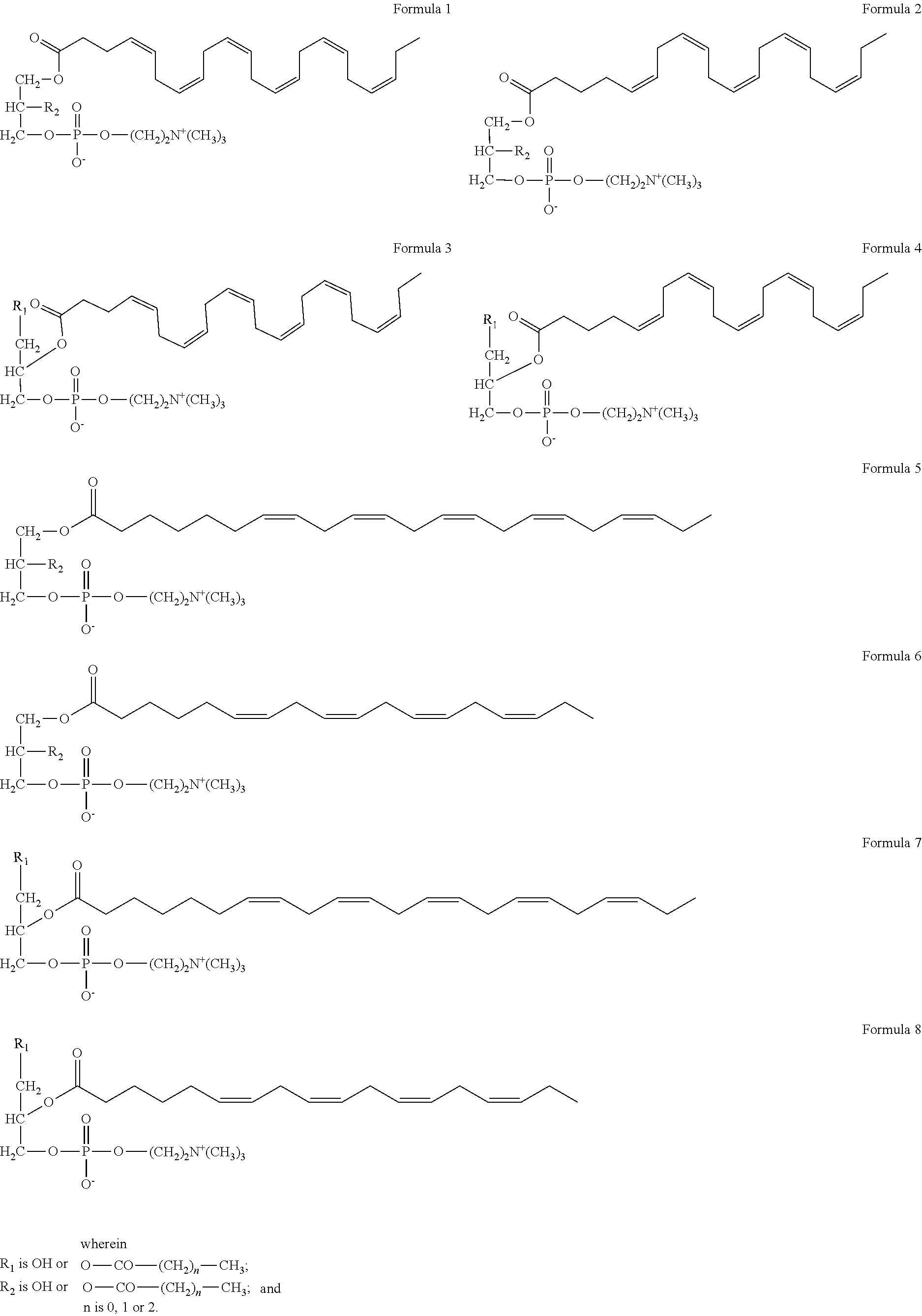

In one embodiment according to the present invention, R₁ is OH and R₂ is OH.

An alternative aspect according to the present invention relates to the first aspect of the present invention wherein R₁ is OH or a protecting group and R₂ is OH or a protecting group. One example of a protective group being O—CO—(CH₂)ₙ—CH₃, wherein n is 0, 1 or 2.

The protecting group is preferably a group which do not interfere with binding to the Mfsd2a transporter and at the same time it blocks migration of the omega-3 (i.e. DHA, EPA, SDA and DPA) acyl group. If the omega-3 fatty acid moiety (e.g. DHA moiety, EPA moiety, SDA moiety and DPA moiety) is positioned on the sn-1 position of the glycerol backbone, the protecting group will typically block migration of the omega-3 fatty acid moiety from the sn-1 position to the sn-2 position. If the omega-3 fatty acid moiety (e.g. DHA moiety) is positioned on the sn-2 position of the glycerol backbone, the protecting group will typically block migration of the omega-3 fatty acid moiety from the sn-2 position to the sn-1 position.

Formula 1 and 3 refers to a compound with an attached DHA moiety. Formula 2 and 4 refers to a compound with an attached EPA moiety. Formula 5 and 7 refers to a compound with an attached n-3 DPA moiety. Formula 6 and 8 refers to a compound with an attached SDA moiety. In practice, the DHA, EPA, DPA and SDA moieties may in principle be replaced by any omega-3 fatty acid as long as the omega-3 fatty acid has 14 or more C-atoms. However, DHA, EPA, DPA and SDA are believed to be of most relevance with respect to human brain health.

An alternative aspect according to the present invention relates to the first aspect of the present invention, wherein the DHA, EPA, DPA and SDA moieties are replaced by any omega-3 moiety; at least i) any omega-3 moiety which has 14 or more C-atoms in its chain or ii) any omega-3 moiety which has a length corresponding to a chain length of 14 or more C-atoms.

An alternative aspect according to the present invention relates to the first aspect of the present invention, wherein the DHA, EPA, DPA and SDA moieties are replaced by DHA, EPA, DPA, ALA and SDA moieties.

In one embodiment according to the present invention, the intravascular administration is intravenous administration. Intravenous administration may be conducted by injections, e.g. with a syringe at higher pressures, or by infusions, e.g. using only the pressure supplied by gravity.

It has previously been acknowledged that going from oral to intravenous administration often raises a question of patient compliance; and when it comes to intravascular administration, in particular intravenous administration, it is of course an advantage to have as few injections as possible. The surprising results presented herein are based on a single injection.

Thus, in one embodiment according to the present invention, the intravenous administration is conducted by one or more injections, preferably less than 5 injections, more preferably less than 3 injections and most preferably less than 2 injections such as a single injection. The technical effect of the latter having already been demonstrated in example 2 of the present application.

The one or more active components referred to in the first aspect of the present invention, wherein R₁ is OH and R₂ is OH are all LPC molecules having either a DHA, an EPA, a DPA or a SDA molecule attached to the triacylglycerol moiety of LPC. Technical effect has been demonstrated for LPC-DHA and LPC-EPA. Based on the data presented in WO2018162617 and WO2008068413 it is also believed that similar effects would be obtained for the one or more active components referred to in the first aspect of the present invention where R₁ is O—CO—(CH₂)ₙ—CH₃ and R₂ is O—CO—(CH₂)ₙ—CH₃; and n is 0, 1 or 2, and in particular n=0.

Even though the results presented herein are impressive, the effect may be even further improved e.g. by including a pharmaceutically acceptable carrier. Liposomes may e.g. be suitable carriers for the oily constituents of the present invention by providing a hydrophobic interior for the oily substance and a hydrophilic exterior facing the hydrophilic environment. Further, it is also known that LPC is typically associated to proteins, such as albumin, in the blood to reduce the effective concentration of LPC. Thus, in one embodiment according to the present invention, the pharmaceutical composition also comprises a protein, such as albumin, which is suitable to reduce the effective concentration of the one or more active components when administered intravascularly or intravenously.

The pharmaceutical composition of the present invention may or may not comprise one or more solvents, such as ethanol and/or water. If the composition comprises one or more solvents, the amount of the one or more active components in the composition may be referred to as % by dry-weight of the composition. However, if the composition does not comprise one or more solvents, the amount of the one or more active components in the composition may be referred to as % by weight of the composition.

In one embodiment according to the present invention, the pharmaceutical composition may comprise a combination of two or more of the one or more active components. One of the active components may have a DHA moiety attached to the glycerol backbone and another active component may have an EPA moiety attached to the glycerol backbone.

Thus, in one embodiment according to the present invention, the pharmaceutical composition comprises a combination of two or more of the one or more active components. One of the active components having a DHA moiety attached to the glycerol backbone and the other active component having an EPA moiety attached to the glycerol backbone. In a preferred embodiment, there is a specific molar ratio of the active components having a DHA moiety attached to the glycerol backbone and the active components having a EPA moiety attached to the glycerol backbone.

The molar ratio of the active components having a DHA moiety attached to the glycerol backbone:the active components having a EPA moiety attached to the glycerol backbone preferably being in the range 1:1 to 10:1, such as in the range 1:1 to 7:1, or in the range 1:1 to 5:1, or in the range 1:1 to 3:1. In another embodiment according to the present invention, the molar ratio of the active components having a EPA moiety attached to the glycerol backbone:the active components having a DHA moiety attached to the glycerol backbone preferably being in the range 1:1 to 10:1, such as in the range 1:1 to 7:1, or in the range 1:1 to 5:1, or in the range 1:1 to 3.1.

Reference is made to the following example illustrating how the molar ratio is to be calculated. If a composition comprises 10 mol LPC-DHA and 2 mol LPC-EPA, then the molar ratio of the active components having a DHA moiety attached to the glycerol backbone and the active components having a EPA moiety attached to the glycerol backbone is 10:2, i.e. 5:1. If not specified otherwise, the number of moles of LPC-EPA is the number of moles 1-LPC-EPA+the number of moles 2-LPC-EPA and the number of moles of LPC-DHA is the number of moles 1-LPC-DHA+the number of moles 2-LPC-DHA.

It has previously been discussed that the position of the omega-3 fatty acid moiety on the glycerol backbone may affect the uptake of that fatty acid into the brain. Thus, in one embodiment according to the present invention, the listed omega-3 fatty acid moieties are bond to sn1 position of the glycerol backbone. In another embodiment according to the present invention, the listed omega-3 fatty acid moieties are bond to sn2 position of the glycerol backbone. In an alternative embodiment according to the present invention, there is a specific molar ratio of the active components having an omega-3 fatty acid moiety bound to sn1 position of the glycerol backbone and the active components having an omega-3 fatty acid moiety bound to sn1 position of the glycerol backbone. The molar ratio of the active components having an omega-3 fatty acid moiety bound to sn2 position of the glycerol backbone:the active components having an omega-3 fatty acid moiety bound to sn1 position of the glycerol backbone preferably being in the range 1:8 to 18:1, such as in the range 1:8 to 15:1 or in the range 1:8 to 10:1.

Reference is made to the following example illustrating how the molar ratio is to be calculated. If a composition comprises 5 mol 2-LPC-DHA, 5 mol 2-LPC-EPA and 2 mol 1-LPC-DHA, then the molar ratio of the active components having an omega-3 fatty acid moiety bound to sn1 position of the glycerol backbone:the active components having an omega-3 fatty acid moiety bound to sn2 position of the glycerol backbone is 10:2, i.e. 5:1.

A second aspect the present invention relates to the pharmaceutical composition according to the first aspect of the present invention for use as a medicament, wherein the pharmaceutical composition is to be administered by intravascular administration, such as intravenous administration.

A third aspect the present invention relates to the pharmaceutical composition according to the first aspect of the present invention for use in prophylaxis and/or therapy, wherein the pharmaceutical composition is to be administered by intravascular administration, such as intravenous administration.

A fourth aspect the present invention relates to the pharmaceutical composition according to the first aspect of the present invention for use in prophylaxis and/or therapy of a condition which would benefit from increased levels of cerebral EPA levels, wherein the pharmaceutical composition is to be administered by intravascular administration, such as intravenous administration.

Depression is an example of an indication that may benefit from increased levels of cerebral EPA levels.

According to the American psychiatric association, depression (major depressive disorder) is a common and serious medical illness that negatively affects how you feel, the way you think and how you act. Depression causes feelings of sadness and/or a loss of interest in activities once enjoyed. It can lead to a variety of emotional and physical problems and can decrease a person's ability to function at work and at home.

Depression symptoms can vary from mild to severe and can include:
Feeling sad or having a depressed mood;
Loss of interest or pleasure in activities once enjoyed;
Changes in appetite—weight loss or gain unrelated to dieting;
Trouble sleeping or sleeping too much;
Loss of energy or increased fatigue;
Increase in purposeless physical activity (e.g., hand-wringing or pacing) or slowed movements and speech (actions observable by others);
Feeling worthless or guilty;
Difficulty thinking, concentrating or making decisions;
Thoughts of death or suicide;

Symptoms must last at least two weeks for a diagnosis of depression.

A fifth aspect the present invention relates to the pharmaceutical composition according to the first aspect of the present invention for use in prophylaxis and/or therapy of a condition which would benefit from increased levels of cerebral DHA levels, wherein the pharmaceutical composition is to be administered by intravascular administration, such as intravenous administration.

In one embodiment according to the fifth aspect of the present invention, the condition which would benefit from increased levels of cerebral DHA levels is a neurological condition.

In another embodiment according to the fifth aspect of the present invention, the neurological condition is depression, Schizophrenia, Alzheimer's disease, Parkinson's disease or traumatic brain injury.

According to the American psychiatric association, schizophrenia is a chronic brain disorder. When schizophrenia is active, symptoms can include delusions, hallucinations, trouble with thinking and concentration, and lack of motivation. However, with treatment, most symptoms of schizophrenia will greatly improve.

When the disease is active, it can be characterized by episodes in which the patient is unable to distinguish between real and unreal experiences. As with any illness, the severity, duration and frequency of symptoms can vary; however, in persons with schizophrenia, the incidence of severe psychotic symptoms often decreases during a patient's lifetime. Symptoms fall into several categories:
Positive psychotic symptoms: Hallucinations, such as hearing voices, paranoid delusions and exaggerated or distorted perceptions, beliefs and behaviors.
Negative symptoms: A loss or a decrease in the ability to initiate plans, speak, express emotion or find pleasure.
Disorganization symptoms: Confused and disordered thinking and speech, trouble with logical thinking and sometimes bizarre behavior or abnormal movements.
Impaired cognition: Problems with attention, concentration, memory and declining educational performance.

Parkinson's disease (PD) is a long-term degenerative disorder of the central nervous system that mainly affects the motor system. As the disease worsens, non-motor symptoms become more common. The symptoms usually emerge slowly. Early in the disease, the most obvious symptoms are shaking, rigidity, slowness of movement, and difficulty with walking. Thinking and behavioral problems may also occur. Dementia becomes common in the advanced stages of the disease. Depression and anxiety are also common, occurring in more than a third of people with PD. Other symptoms include sensory, sleep, and emotional problems. The main motor symptoms are collectively called “parkinsonism”, or a “parkinsonian syndrome”.

In a preferred embodiment according to the fifth aspect of the present invention, the condition which would benefit from increased levels of cerebral DHA levels is traumatic brain injury.

Traumatic brain injury (TBI) is a head injury caused by trauma to the brain. The damage can be confined to one area of the brain (focal) or involve more than one area of the brain (diffuse). TBI can be mild, moderate or severe. While some symptoms appear immediately, others do not appear until days, weeks, months or even years after the TBI event(s). Symptoms of mild TBI include headache, confusion, dizziness, blurred vision, changes in mood, and impairment in cognitive function, such as memory, learning, and attention. Symptoms of moderate to severe TBI include, in addition to those observed for mild TBI, nausea, convulsions or seizures, slurring of speech, numbness of extremities, and loss of coordination.

Traditional concepts of TBI also involve primary and secondary injury phases. The primary injury is represented by the moment of impact, resultant from the impartation of kinetic energy and force vectors in either a linear acceleration-deceleration or rotatory fashion, or a combination of both. In addition to the motion of the brain within the cerebrospinal fluid space, brain contact with underlying irregular surfaces of the skull, the establishing of micro-vacuum phenomena within the cerebral tissue, and the tearing and mechanical injury to neurons and particularly their projections can result in both local and remote damage. At the clinical level, treatment attempts to minimize secondary injury by preventing or treating hypotension, hypoxia, and edema.

A tertiary phase of TBI includes what are now recognized as ongoing abnormalities in glucose utilization, cellular metabolism, as well as membrane fluidity, synaptic function, and structural integrity (Hovda, Crit Care Med. 35:663-4 (2007); Aoyama et al, Brain Res. 1230:310-9 (2008), published electronically Jul. 9, 2008). In general, axon membranes are injured, ionic leakage occurs, and axonal transport is interrupted in a progressive manner. This concept is reinforced by recent autopsy findings in professional contact sports athletes showing multi-focal areas of damaged neurons and their processes, remarkable for tau antibody staining, believed to represent numerous times and regions of injury from multiple concussions (Omalu et al., Neurosurgery 57:128-34 (2005); Omalu et al., Neurosurgery 59:1086-92 (2006)).

Promising results for prophylactic treatment of TBI based on means suitable to increase the levels of DHA in brain have been reported in the prior art (EP2488190).

In a preferred embodiment according to the fifth aspect of the present invention, the condition which would benefit from increased levels of cerebral DHA levels is traumatic brain injury and the pharmaceutical composition is administered in combination with i) progestogen or a prodrug thereof; and/or ii) estrogen or a prodrug thereof.

In a preferred embodiment according to the fifth aspect of the present invention, the condition which would benefit from increased levels of cerebral DHA levels is traumatic brain injury and the traumatic brain injury is from a closed head injury.

In one embodiment according to the fifth aspect of the present invention, the condition which would benefit from increased levels of cerebral DHA levels is post-traumatic stress disorder (PTSD) or anxiety.

A sixth aspect the present invention relates to the pharmaceutical composition according to the first aspect of the present invention for use in prophylaxis and/or therapy of a condition which would benefit from increased levels of cerebral DPA levels, wherein the pharmaceutical composition is to be administered by intravascular administration, such as intravenous administration.

A seventh aspect the present invention relates to the pharmaceutical composition according to the first aspect of the present invention for use in prophylaxis and/or therapy of a condition which would benefit from increased levels of cerebral SDA levels, wherein the pharmaceutical composition is to be administered by intravascular administration, such as intravenous administration.

It is to be understood that a condition which e.g. would benefit from increased levels of cerebral DHA levels may be treated by increasing the cerebral EPA levels since at least part of the EPA in the brain may be converted to DPA.

An eighth aspect of the present invention relates to the pharmaceutical composition according to the first aspect of the present invention, wherein R₁ and R₂ is OH, for use in prophylaxis and/or therapy; wherein the pharmaceutical composition is to be administered by intravascular administration, such as intravenous administration.

An ninth aspect of the present invention relates to the pharmaceutical composition according to the first aspect of the present invention, wherein R₁ and R₂ is OH, for use in prophylaxis and/or therapy of a condition which would benefit from increased cerebral DHA levels, wherein the pharmaceutical composition is to be administered by intravascular administration, such as intravenous administration.

In one embodiment according to the ninth aspect of the present invention, the condition which would benefit from increased cerebral DHA levels is a neurological condition, the neurological condition preferably being traumatic brain injury.

In one embodiment according to the ninth aspect of the present invention, the condition which would benefit from increased cerebral DHA levels is post-traumatic stress disorder (PTSD) or anxiety.

Posttraumatic stress disorder (PTSD) is a mental disorder that can develop after a person is exposed to a traumatic event, such as sexual assault, warfare, traffic collisions, or other threats on a person's life. Symptoms may include disturbing thoughts, feelings, or dreams related to the events, mental or physical distress to trauma-related cues, attempts to avoid trauma-related cues, alterations in how a person thinks and feels, and an increase in the fight-or-flight response. These symptoms last for more than a month after the event. Young children are less likely to show distress, but instead may express their memories through play. A person with PTSD may be at a higher risk for suicide and intentional self-harm.

In one embodiment according to any one of aspects 2-9, the pharmaceutical composition is to be administered to a subject who is at risk of traumatic brain injury. The pharmaceutical composition is preferably administered in a prophylactically effective amount for a sufficient time period prior to engagement in an activity associated with a risk of traumatic brain injury to reduce the risk of pathological effects of traumatic brain injury. The traumatic head injury may be

A tenth aspect the present invention relates to the pharmaceutical composition according to the first aspect of the present invention for use to treat, prevent, or improve cognition and/or a cognitive disease, disorder or impairment (memory, concentration, learning (deficit)), or to treat or prevent neurodegenerative disorders; wherein the pharmaceutical composition is to be administered by intravascular administration, such as intravenous administration.

In some embodiments, the cognitive disease, disorder or impairment is selected from Attention Deficit Disorder (ADD), Attention Deficit Hyperactivity Disorder (ADHD), autism/autism spectrum disorder (ASD), (dyslexia, age-associated memory impairment and learning disorders, amnesia, mild cognitive impairment, cognitively impaired non-demented, pre-Alzheimer's disease, Alzheimer's disease, epilepsy, Pick's disease, Huntington's disease, Parkinson disease, Lou Gehrig's disease, pre-dementia syndrome, Lewy body dementia, dentatorubropallidoluysian atrophy, Freidreich's ataxia, multiple system atrophy, types 1, 2, 3, 6, 7 spinocerebellar ataxia, amyotrophic lateral sclerosis, familial spastic paraparesis, spinal muscular atrophy, spinal and bulbar muscular atrophy, age-related cognitive decline, cognitive deterioration, moderate mental impairment, mental deterioration as a result of ageing, conditions that influence the intensity of brain waves and/or brain glucose utilization, stress, anxiety, concentration and attention impairment, mood deterioration, general cognitive and mental well-being, neurodevelopmental, neurodegenerative disorders, hormonal disorders, neurological imbalance or any combinations thereof. In a specific embodiment, the cognitive disorder is memory impairment.

An eleventh aspect the present invention relates to the pharmaceutical composition according to the first aspect of the present invention for use to treat or prevent a cardiovascular disorder or metabolic syndrome; wherein the pharmaceutical composition is to be administered by intravascular administration, such as intravenous administration.

In some embodiments, the cardiovascular disorder is selected from atherosclerosis, arteriosclerosis, coronary heart (carotid artery) disease (CHD or CAD), acute coronary syndrome (or ACS), valvular heart disease, aortic and mitral valve disorders, arrhythmia/atrial fibrillation, cardiomyopathy and heart failure, angina pectoris, acute myocardial infarction (or AMI), hypertension, orthostatic hypotension, shock, embolism (pulmonary and venous), endocarditis, diseases of arteries, the aorta and its branches, disorders of the peripheral vascular system (peripheral arterial disease or PAD), Kawasaki disease, congenital heart disease (cardiovascular defects) and stroke (cerebrovascular disease), dyslipidemia, hypertriglyceridemia, hypertension, heart failure, cardiac arrhythmias, low HDL levels, high LDL levels, stable angina, coronary heart disease, acute myocardial infarction, secondary prevention of myocardial infarction, cardiomyopathy, endocarditis, type 2 diabetes, insulin resistance, impaired glucose tolerance, hypercholesterolemia, stroke, hyperlipidemia, hyperlipoproteinemia, chronic kidney disease, intermittent claudication, hyperphosphatemia, omega-3 deficiency, phospholipid deficiency, carotid atherosclerosis, peripheral arterial disease, diabetic nephropathy, hypercholesterolemia in HIV infection, acute coronary syndrome (ACS), non-alcoholic fatty liver disease/non-alcoholic steatohepatitis (NAFLD/NASH), arterial occlusive diseases, cerebral atherosclerosis, arteriosclerosis, cerebrovascular disorders, myocardial ischemia, coagulopathies leading to thrombus formation in a vessel and diabetic autonomic neuropathy.

A twelfth aspect the present invention relates to the pharmaceutical composition according to the first aspect of the present invention for use to inhibit, prevent, or treat inflammation or an inflammatory disease; wherein the pharmaceutical composition is to be administered by intravascular administration, such as intravenous administration.

In some embodiments, the inflammation or inflammatory disease is selected from organ transplant rejection; reoxygenation injury resulting from organ transplantation (see Grupp et al., J. Mol. Cell. Cardiol. 31: 297-303 (1999)) including, but not limited to, transplantation of the following organs: heart, lung, liver and kidney; chronic inflammatory diseases of the joints, including arthritis, rheumatoid arthritis, osteoarthritis and bone diseases associated with increased bone resorption; inflammatory bowel diseases (IBD) such as ileitis, ulcerative colitis (UC), Barrett's syndrome, and Crohn's disease (CD); inflammatory lung diseases such as asthma, acute respiratory distress syndrome (ARDS), and chronic obstructive pulmonary disease (COPD); inflammatory diseases of the eye including corneal dystrophy, trachoma, onchocerciasis, uveitis, sympathetic ophthalmitis and endophthalmitis; chronic inflammatory diseases of the gum, including gingivitis and periodontitis; inflammatory diseases of the kidney including uremic complications, glomerulonephritis and nephrosis; inflammatory diseases of the skin including sclerodermatitis, psoriasis and eczema; inflammatory diseases of the central nervous system, including chronic demyelinating diseases of the nervous system, multiple sclerosis, AIDS-related neurodegeneration and Alzheimer's disease, infectious meningitis, encephalomyelitis, Parkinson's disease, Huntington's disease, Epilepsy, amyotrophic lateral sclerosis and viral or autoimmune encephalitis, preeclampsia; chronic liver failure, brain and spinal cord trauma, and cancer. The inflammatory disease can also be a systemic inflammation of the body, exemplified by gram-positive or gram negative shock, hemorrhagic or anaphylactic shock, or shock induced by cancer chemotherapy in response to proinflammatory cytokines, e.g., shock associated with proinflammatory cytokines. Such shock can be induced, e.g., by a chemotherapeutic agent that is administered as a treatment for cancer. Other disorders include depression, obesity, allergic diseases, acute cardiovascular events, muscle wasting diseases, and cancer cachexia. Also, inflammation that results from surgery and trauma can be treated with the phospholipid compositions.

A thirteenth aspect the present invention relates to the pharmaceutical composition according to the first aspect of the present invention for use to treat a disease or condition associated with red blood cells and cell membranes, and in particular a disease or conditions associated with an abnormality in red blood cells of cell membranes; wherein the pharmaceutical composition is to be administered by intravascular administration, such as intravenous administration.

In some embodiments, the condition or disease is sickle cell disease, sickle cell anemia, or sickle cell trait. In some embodiments, the condition or disease is thalassemia (alpha-, beta- or delta-), thalassemia in combination with a hemoglobinopathy (Hemoglobin E, Hemoglobin S, or Hemoglobin C), splenomegaly, or membrane abnormities such as acanthocytes or spur/spike cells, codocytes (target cells), echinocytes (burr cells), elliptocytes and ovalocytes, spherocytes, stomatocytes (mouth cells) and degmacytes (“bite cells”).

In one embodiment according to any one of aspects 2-13, the pharmaceutical composition is to be administered to a subject of less than 10 years of age, such as less than 1 year of age, less than 1 month of age, or a newborn.

In one embodiment according to any one of aspects 2-13, the pharmaceutical composition is to be administered to a subject of more than 60 years of age, such as more than 70 years of age, more than 80 months of age, or to an elderly subject.

In one embodiment according to any one of aspects 2-13, the pharmaceutical composition is to be administered to a subject, wherein the subject is from about 10 to 20 years of age, from about 20 to 50 years of age from about 50 to 100 years of age, from about 60 to 100 years of age or from about 70 to 100 years of age.

In one embodiment according to any one of aspects 2-13, the pharmaceutical composition is to be administered to a subject, wherein the subject is female.

In one embodiment according to any one of aspects 2-13, the pharmaceutical composition is to be administered to a subject, wherein the subject is male.

In one embodiment according to the present invention, traumatic brain injury does not include brain injury induced by ischemia/reperfusion.

In some embodiments, the closed head injury is a concussion or contusion. A subject at risk for such injury can include, among others, a subject participating in an athletic event with occurrence of concussions. Exemplary subjects in this category include, among others, football players, boxers, and hockey players.

An alternative aspect of the present invention relates to a method for administering the pharmaceutical composition according to the first aspect of the present invention to a subject, wherein the pharmaceutical composition is administered by intravascular administration, such as intravenous administration.

A further alternative aspect of the present invention relates to a method for prophylactic or therapeutic treatment of a subject, the method comprising the following steps:
administering the pharmaceutical composition according to the first aspect of the present invention to the subject by intravascular administration, such as intravenous administration.

A further alternative aspect of the present invention relates to a method for prophylactic or therapeutic treatment of a subject suffering from a condition which would benefit from increased levels of cerebral EPA levels, the method comprising the following steps:
administering the pharmaceutical composition according to the first aspect of the present invention to the subject by intravascular administration, such as intravenous administration.

In one embodiment, the condition which would benefit from increased levels of cerebral EPA levels is depression.

A further alternative aspect of the present invention relates to a method for prophylactic or therapeutic treatment of a subject suffering from a condition which would benefit from increased levels of cerebral DHA levels, the method comprising the following steps:
administering the pharmaceutical composition according to the first aspect of the present invention to the subject by intravascular administration, such as intravenous administration.

In one embodiment, the condition which would benefit from increased levels of cerebral DHA levels is a neurological condition. The neurological condition preferably being selected from the group consisting of depression, Schizophrenia, Alzheimer's disease, Parkinson's disease or traumatic brain injury, and in particular traumatic brain injury.

In another embodiment, the condition which would benefit from increased levels of cerebral DHA levels is post-traumatic stress disorder (PTSD) or anxiety.

In another embodiment, the pharmaceutical composition is administered in combination with i) progestogen or a prodrug thereof; and/or ii) estrogen or a prodrug thereof.

A further alternative aspect of the present invention relates to a method for reducing the risk of pathological effects of TBI, comprising:
administering the pharmaceutical composition according to the first aspect of the present invention to a subject who is at risk of TBI;

wherein
the pharmaceutical composition is administered by intravascular, and in particular intravenous administration;
the pharmaceutical composition is administered in a prophylactically effective amount for a sufficient time period prior to engagement in an activity associated with a risk of TBI to reduce the risk of pathological effects of TBI.

Having generally described this invention, a further understanding can be obtained by reference to certain specific examples, which are provided herein for purposes of illustration only, and are not intended to be limiting unless otherwise specified.

### EXAMPLES

### Example 1: Preparation of Intravenous Formulations

Materials

Intralipid (IV dose)

| | |
|---|---|
| Source/supplier | Sigma Aldrich |
| CAS number | 68890-65-3 |
| Physical form/appearance | White liquid |
| Constituents/concentrations | 20% fat emulsion |

[¹⁴C]-LPC-EPA

| Structure | 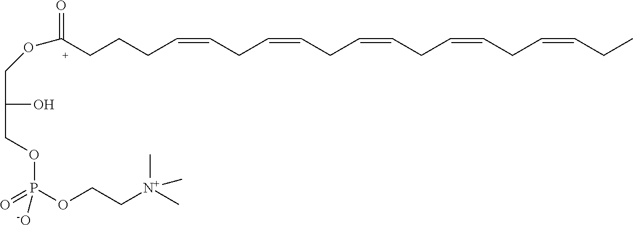 |
|---|---|
| Source/supplier | Pharmaron UK |
| Physical form/appearance | Ethanolic solution |
| | 3 mCi/g [2.361 mCi/mL accounting for specific gravity of ethanol (0.787)] |
| Molecular weight | 543.5 |
| Specific activity | 57 mCi/mmol (2.11 GBq/mmol) |
| Radiochemical purity | 96.3% |

[¹⁴C]-LPC-DHA

| Structure | 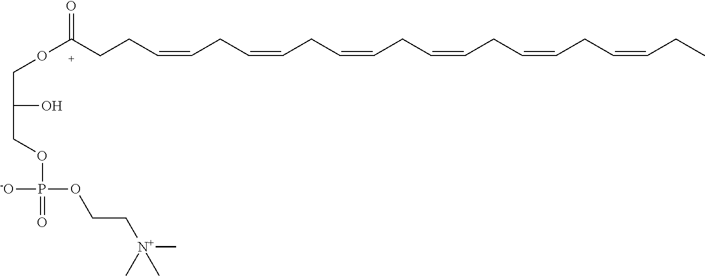 |
|---|---|
| Source/supplier | Pharmaron UK |
| Physical form/appearance | Ethanolic solution |
| | 3 mCi/g [2.361 mCi/mL accounting for specific gravity of ethanol (0.787)] |
| Molecular weight | 569.6 |
| Specific activity | 58 mCi/mmol (2.15 GBq/mmol) |
| Radiochemical purity | 98.0% |

[¹⁴C]-LPC-DHA Formulation, Herein Referred to as Formulation A

The formulation that was later administered intravenously was prepared according to the following target specifications:

| | |
|---|---|
| Dose regimen and route | Single IV |
| Dose level | 190 mg/kg of intralipid + about 1.5 |
| | mg/kg radiolabeled compound |
| Radioactive dose | 155 μCi/kg |
| Dose volume | 1 mL/kg |
| Dose concentration | 190 mg/mL @ 5: 95 ethanolic [¹⁴C]- |
| | LPC-DHA: intralipid (20%) v/v |
| Dose vehicle | 5 : 95 v/v ethanol : injectable intralipid |
| | (20% emulsion) |
| Formulation specific | 3 mCi/g |
| radioactivity | |
| Dose formulation | 1.5215 mg/mL |
| radioactive | |
| concentration | |

The [¹⁴C]-LPC-DHA was mixed with the intralipid formulation to yield a dose formulation containing phospholipids at a final concentration of 190 mg/kg and the [¹⁴C]-LPC-DHA at a concentration of about 1.5 mg/kg (155 Ki/kg) as follows:

0.394 mL of ethanolic [¹⁴C]-LPC-DHA (2361 μCi/mL) was dispensed into a 20 mL glass vial and reduced to a final volume of approximately 0.30 mL under a flow of nitrogen at ambient temperature. 5.70 mL of 20% intralipid was added to the concentrated ethanolic [¹⁴C]-LPC-DHA solution and gently vortex mixed to ensure homogeneity.

[¹⁴C]-LPC-EPA Formulation, Herein Referred to as Formulation B

The formulation that was later administered intravenously was prepared according to the following target specifications:

| | |
|---|---|
| Dose regimen and route | Single IV |
| Dose level | 190 mg/kg of intralipid + about 1.5 |
| | mg/kg radiolabeled compound |
| Radioactive dose | 155 μCi/kg |
| Dose volume | 1 mL/kg |
| Dose concentration | 190 mg/mL @ 5: 95 ethanolic [¹⁴C]- |
| | LPC-EPA : intralipid (20%) v/v |
| Dose vehicle | 5 : 95 v/v ethanol: injectable intralipid |
| | (20% emulsion) |
| Formulation specific | 3 mCi/g |
| radioactivity | |
| Dose formulation | 1.4773 mg/mL |
| radioactive | |
| concentration: | |

The [¹⁴C]-LPC-EPA was mixed with the intralipid formulation to yield a dose formulation containing phospholipids at a final concentration of 190 mg/kg and the LPC-EPA at a concentration of about 1.5 mg/kg (155 Ki/kg) as follows:

0.394 mL of ethanolic [¹⁴C]-LPC-EPA (2361 Ki/mL) was dispensed into a 20 mL glass vial and reduced to a final volume of approximately 0.30 mL under a flow of nitrogen at ambient temperature. 5.70 mL of 20% intralipid was added to the concentrated ethanolic [¹⁴C]-LPC-EPA solution and gently vortex mixed to ensure homogeneity.

### Example 2: Uptake of LPC in Tissues—Intravenous Administration

16 male Sprague Dawley rats, in the weight range of 213-289 g and approximately 7-8 weeks old at the time of dose administration were housed in polypropylene cages and remained therein except for a short period during dosing. The room in which the animals were located was thermostatically monitored and data recorded continually (generally the temperature range was 21±2° C.; humidity range 55±10%) and exposed to 12 hours fluorescent lighting and 12 hours dark per day. Animals were equilibrated under standard animal house conditions for a minimum of 3 days prior to use. The health status of the animals was monitored throughout this period and the suitability of each animal for experimental use was confirmed before use.

A pellet diet (RM1 (E) SQC, Special Diets Services, Witham, Essex, UK) and water (from the domestic water supply) was available ad libitum throughout the holding, acclimatization and post-dose periods.

The 16 rats received a single intravenous administration of either formulation A or formulation B (eight per formulation) according to the dosage specification specified in example 1. Each rat was weighed prior to dose administration and the individual doses administered were calculated based on the bodyweight and the specified dose volume.

Dose utensils for intravenous administration consisted of a hypodermic syringe and needle. The dose was administered directly into a tail vein as a slow bolus over 30 seconds.

After single intravenous administration of formulation A or B to the 16 male rats, a single rat was euthanized by overdose of carbon dioxide gas at each of the following times: 0.5, 3, 8, 24, 72, 96, 168 and 336 hours post-dose.

Each carcass was snap frozen in a hexane/solid carbon dioxide mixture immediately after collection and were then stored at approximately −20° C., pending analysis by QWBA (Quantitative whole body autoradiography).

The frozen carcasses were subjected to QWBA using procedures based on the work of Ullberg (Acta. Radiol. Suppl 118, 22 31, 1954). Sections were presented at up to five different levels of the rat body to include between 30 and 40 tissues (subject to presence of sufficient radioactivity) of which the uptake in brain, blood, kidney and spleen are disclosed herein.

The freeze-dried whole body autoradiography sections were exposed to phosphor-storage imaging plates and incubated at ambient temperature in the dark for a minimum of five days.

A series of calibrated auto radiographic [¹⁴C] microscales containing known amounts of radioactivity (nCi/g, produced by Perkin Elmer) were exposed alongside the animal sections on each plate.

Distribution of radioactivity was determined in tissues and microscales and quantified using a Fuji FLA-5100 fluorescent image analysing system and associated Tina (version 2.09) and SeeScan (version 2.0) software.

A representative background radioactivity measurement was taken for each exposure plate used. The limit of accurate quantification was considered to be the lowest [14C] microscale visible. A standard curve was produced from the microscales using Seescan and from which tissue concentrations of radioactivity were determined (nCi/g). For calculation of the weight equivalent/g data, the nCi/g data was divided by the relevant specific activity (nCi/μg).

Table 1.1 shows total amounts of radioactivity in tissues (blood, brain, kidney, spleen) following a single intravenous administration of [¹⁴C]-LPC-DHA to male albino rats at a target dose of 190 mg/kg. The results are also presented in FIG. 1-4.

Table 1.2 demonstrate concentration of radioactivity in all tissues (expressed as μg equivalents/g) following a single intravenous administration of [¹⁴C]-LPC-DHA to male albino rats at a target dose of 190 mg/kg.

Table 2.1 shows the total amounts of radioactivity in tissues (blood, brain, kidney, spleen) following a single intravenous administration of [14C]-LPC-EPA to male albino rats at a target dose of 190 mg/kg. The results are also presented in FIG. 5-8.

Table 2.2 demonstrate concentration of radioactivity in all tissues (expressed as μg equivalents/g) following a single intravenous administration of [¹⁴C]-LPC-EPA to male albino rats at a target dose of 190 mg/kg.

TABLE 1.1

| | | Time after administration (hrs) | | | | | | | |
|---|---|---|---|---|---|---|---|---|---|
| DHA-LPC | | 0.5 | 3 | 8 | 24 | 72 | 96 | 168 | 336 |
| % of | Blood | 4.40 | 1.13 | 0.901 | 0.870 | 0.425 | 0.331 | 0.136 | 0.153 |
| dose/tissue | Brain | 0.838 | 0.755 | 0.684 | 0.751 | 1.22 | 0.970 | 0.880 | 1.11 |
| | Kidney (whole) | 2.00 | 1.76 | 1.50 | 1.28 | 0.711 | 0.714 | 0.321 | 0.098 |
| | Spleen | 0.235 | 0.145 | 0.155 | 0.168 | 0.107 | 0.075 | 0.037 | 0.016 |

TABLE 1.2
Concentration of radioactivity in all tissues (expressed as μg equivalents/g) following a single
intravenous administration of [¹⁴C]-LPC-DHA to male albino rats at a target dose of 190 mg/kg

| | | Animal no: | | | | | | | |
|---|---|---|---|---|---|---|---|---|---|
| | | 48M | 49M | 50M | 51M | 52M | 45M | 46M | 47M |
| | | Time-point: | | | | | | | |
| Tissue type | Tissue | 0.5 h | 3 h | 8 h | 24 h | 72 h | 96 h | 168 h | 336 h |
| Alimentary | Caecum contents | 0.009 | 0.148 | 0.433 | 0.260 | 0.102 | 0.047 | 0.021 | 0.007 |
| canal | Caecum mucosa | 1.24 | 1.80 | 1.58 | 1.33 | 1.27 | 0.603 | 0.352 | 0.238 |
| | Large intestine contents | 0.005 | 0.021 | 1.44 | 0.356 | 0.225 | 0.093 | 0.042 | 0.019 |
| | Large intestine mucosa | 1.03 | 0.764 | 0.766 | 1.83 | 1.19 | 0.729 | 0.271 | 0.187 |
| | Small intestine contents | 1.86 | 0.852 | 0.235 | 0.311 | 0.140 | 0.073 | 0.082 | 0.028 |
| | Small intestine mucosa | 7.84^{†} | 4.59 | 3.04 | 3.29 | 1.13 | 0.980 | 0.549 | 0.167 |
| | Stomach contents | 0.005 | 0.006 | BLQ | 0.015 | 0.013 | BLQ | 0.008 | 0.005 |
| | Forestomach mucosa | 0.961 | 0.610 | 0.392 | 0.649 | 0.464 | 0.388 | 0.405 | 0.179 |
| | Glandular stomach mucosa | 1.71 | 1.35 | 1.213 | 1.11 | 0.740 | 0.575 | 0.466 | 0.221 |
| CNS | Brain | 2.37 | 2.13 | 1.90 | 2.10 | 3.41 | 2.80 | 2.49 | 3.12 |
| | Choroid plexus | 0.867 | 2.23 | 2.91 | NS | 1.70 | NS | 0.440 | 0.686 |
| | Spinal cord | 2.10 | 1.86 | 2.06 | 2.33 | 4.70 | 3.28 | 3.07 | 4.05 |
| | Spinal nerve | 15.3^{†} | 9.99 | 5.94^{†} | 4.62 | 2.17 | 4.09 | 0.942 | 1.53 |
| Connective | Bone | 0.077 | 0.093 | 0.048 | 0.037 | 0.061 | 0.026 | 0.041 | 0.012 |
| Dermal | Skin | 0.887 | 0.344 | 0.355 | 0.331 | 0.450 | 0.352 | 0.381 | 0.308 |
| Endocrine | Adrenal gland | 1.38 | 1.75 | 1.39 | 1.83 | 2.27 | 2.01 | 1.08 | 0.606 |
| | Pineal body | 0.980 | 1.64 | 2.03 | 1.75 | 3.26 | 1.85 | NS | 2.53 |
| | Pituitary gland | 3.95 | 7.39^{†} | 2.36 | 2.38 | 4.92 | 3.38 | 2.09 | 1.25 |
| | Thyroid gland | 1.02 | 1.24 | 1.35 | 1.14 | 0.780 | 0.759 | 0.237 | 0.210 |
| Excretory/ | Liver | 11.2^{†} | 10.6^{†} | 8.83^{†} | 6.32^{†} | 2.76 | 2.11 | 1.06 | 0.436 |
| metabolic | Kidney: Cortex | 5.77^{†} | 5.86^{†} | 4.87 | 4.22 | 1.98 | 1.76 | 0.952 | 0.309 |
| | Kidney: Medulla | 3.01 | 2.18 | 1.85 | 1.72 | 1.05 | 1.22 | 0.484 | 0.145 |
| | Kidney: Whole | 4.10 | 3.59 | 3.02 | 2.59 | 1.44 | 1.49 | 0.659 | 0.199 |
| | Urinary bladder contents | 2.15 | NS | NS | 0.135 | NS | 0.087 | 0.041 | 0.005 |
| | Urinary bladder wall | 0.869 | NS | 0.296 | 0.595 | 0.820 | 0.529 | 0.317 | 0.196 |
| Exocrine | Ex-orbital lachrymal gland | 0.867 | 0.898 | 0.864 | 0.826 | 0.989 | 0.658 | 0.816 | 0.223 |
| | Harderian gland | 2.47 | 2.78 | 2.71 | 1.89 | 0.615 | 0.440 | 0.226 | 0.092 |
| | Pancreas | 0.677 | 1.08 | 1.03 | 1.20 | 1.42 | 1.14 | 0.719 | 0.509 |
| | Preputial gland | 6.09 | 5.18 | NS | 10.1^{†} | 21.4^{†} | 5.67^{†} | 7.63 | NS |
| | Salivary gland | 2.10 | 1.70 | 1.73 | 1.33 | 1.46 | 1.04 | 0.490 | 0.339 |
| Fatty | Fat: Brown | 1.17 | 1.57 | 1.64 | 1.23 | 2.10 | 1.34 | 0.818 | 0.691 |
| | Fat: White | 0.115 | 0.664 | 0.838 | 1.05 | 1.64 | 1.30 | 1.01 | 0.766 |
| Ocular | Eye: Lens | BLQ | BLQ | BLQ | 0.031 | 0.031 | 0.045 | 0.019 | 0.019 |
| | Eye: Whole | 0.649 | 0.274 | 0.451 | 0.426 | 0.072 | 0.127 | 0.033 | 0.075 |
| Reproductive | Epididymis | 3.35 | 1.94 | 1.99 | 1.35 | 1.62 | 1.51 | 0.560 | 0.664 |
| | Prostate gland | 0.641 | 0.352 | NS | 0.454 | 0.421 | 0.684 | 0.528 | 0.162 |
| | Seminal vesicles | 1.19 | 0.885 | 0.510 | 0.782 | 0.658 | 0.431 | 0.410 | 0.127 |
| | Testis | 2.57 | 1.33 | 0.735 | 0.538 | 0.477 | 0.450 | 0.296 | 0.282 |
| Respiratory | Lung | 2.27 | 1.34 | 1.24 | 0.761 | 0.570 | 0.313 | 0.254 | 0.159 |
| Skeletal/ | Muscle (skeletal) | 0.326 | 0.331 | 0.328 | 0.486 | 0.738 | 0.663 | 0.620 | 0.639 |
| muscular | Myocardium | 1.02 | 1.96 | 2.31 | 2.59 | 3.89 | 2.59 | 2.35 | 2.17 |
| Vascular/ | Blood (cardiac) | 1.38 | 0.356 | 0.280 | 0.271 | 0.133 | 0.105 | 0.045 | 0.048 |
| lymphatic | Bone marrow | 1.47 | 1.40 | 1.20 | 1.47 | 0.707 | 0.573 | 0.266 | 0.194 |
| | Lymph duct | 2.72 | 0.706 | 0.286 | 0.227 | NS | 0.105 | 0.053 | 0.037 |
| | Lymph node | 1.40 | 0.952 | 0.919 | 0.933 | 0.813 | 0.625 | 0.477 | 0.108 |
| | Spleen | 1.72 | 1.05 | 1.11 | 1.21 | 0.772 | 0.561 | 0.269 | 0.116 |
| | Thymus | 0.952 | 0.839 | 0.706 | 0.629 | 0.480 | 0.300 | 0.135 | 0.073 |

^{†}Above limit of accurate quantification (>5.31 μg equivalents/g)
BLQ Below limit of accurate quantification (<0.004 μg equivalents/g)
NS No sample - tissue not sectioned

TABLE 2.1

| | | Time after administration (hrs) | | | | | | | |
|---|---|---|---|---|---|---|---|---|---|
| EPA-LPC | | 0.5 | 3 | 8 | 24 | 72 | 96 | 168 | 336 |
| % of | Blood | 7.61 | 2.45 | 1.49 | 0.538 | 0.319 | 0.475 | 0.203 | 0.174 |
| dose/tissue | Brain | 0.565 | 0.47 | 0.467 | 0.336 | 0.326 | 0.666 | 0.4 | 0.677 |
| | Kidney (whole) | 4.57 | 2.93 | 1.25 | 0.799 | 0.598 | 0.635 | 0.249 | 0.14 |
| | Spleen | 0.295 | 0.285 | 0.243 | 0.141 | 0.11 | 0.122 | 0.038 | 0.019 |

TABLE 2.2
Concentration of radioactivity in all tissues (expressed as μg equivalents/g) following a single
intravenous administration of [¹⁴C]-LPC-EPA to male albino rats at a target dose of 190 mg/kg.

| | | Animal no: | | | | | | | |
|---|---|---|---|---|---|---|---|---|---|
| | | 32M | 33M | 34M | 35M | 36M | 29M | 30M | 31M |
| | | Time-point: | | | | | | | |
| Tissue type | Tissue | 0.5 h | 3 h | 8 h | 24 h | 72 h | 96 h | 168 h | 336 h |
| Alimentary | Caecum contents | 0.008 | 0.864 | 0.562 | 0.066 | 0.034 | 0.049 | 0.017 | 0.008 |
| canal | Caecum mucosa | 3.30 | 2.62 | 2.18 | 1.81 | 0.662 | 0.890 | 0.217 | 0.194 |
| | Large intestine contents | 0.004 | 0.022 | 1.05 | 0.106 | 0.043 | 0.082 | 0.062 | 0.024 |
| | Large intestine mucosa | 2.15 | 1.86 | 2.12 | 0.933 | 0.546 | 0.536 | 0.338 | 0.182 |
| | Small intestine contents | 2.22 | 0.925 | 0.256 | 0.188 | 0.232 | 0.149 | 0.045 | 0.033 |
| | Small intestine mucosa | 11.7^{†} | 7.76^{†} | 4.34 | 1.92 | 0.840 | 0.864 | 0.235 | 0.171 |
| | Stomach contents | BLQ | 0.006 | 0.004 | BLQ | 0.012 | 0.019 | 0.007 | 0.006 |
| | Forestomach mucosa | 1.49 | 1.09 | 0.477 | 0.311 | 0.348 | 0.589 | 0.258 | 0.143 |
| | Glandular stomach mucosa | 1.60 | 1.93 | 1.56 | 0.774 | 0.642 | 0.716 | 0.278 | 0.176 |
| CNS | Brain | 1.55 | 1.33 | 1.30 | 0.933 | 0.907 | 1.83 | 0.951 | 1.86 |
| | Choroid plexus | 2.35 | NS | NS | 0.384 | 0.502 | 0.673 | 0.237 | 0.370 |
| | Spinal cord | 1.47 | 1.54 | 1.64 | 1.19 | 1.13 | 2.03 | 1.221 | 1.88 |
| | Spinal nerve | 14.7^{†} | 10.0^{†} | 8.50 | 3.35 | 2.38 | 1.36 | 0.977 | 1.84 |
| Connective | Bone | 0.053 | 0.201 | 0.088 | 0.058 | 0.031 | 0.029 | 0.022 | 0.005 |
| Dermal | Skin | 0.898 | 0.600 | 1.23 | 0.313 | 0.296 | 0.489 | 0.229 | 0.270 |
| Endocrine | Adrenal gland | 1.61 | 1.74 | 2.19 | 2.36 | 1.31 | 1.78 | 0.673 | 0.662 |
| | Pineal body | 3.22 | 3.08 | NS | 1.19 | 1.45 | 2.63 | 1.03 | 2.09 |
| | Pituitary gland | 2.37 | 2.70 | 3.70 | 1.38 | 1.34 | 2.11 | 1.00 | 1.20 |
| | Thyroid gland | 1.58 | 1.91 | 2.05 | 1.35 | 0.731 | 1.04 | 0.326 | 0.371 |
| Excretory/ | Liver | 15.3^{†} | 9.25^{†} | 7.70^{†} | 2.96 | 1.47 | 2.02 | 0.628 | 0.386 |
| metabolic | Kidney: Cortex | 13.3^{†} | 10.1^{†} | 3.79 | 2.56 | 1.71 | 1.74 | 0.565 | 0.344 |
| | Kidney: Medulla | 6.46^{†} | 3.51 | 1.81 | 1.10 | 0.942 | 0.959 | 0.342 | 0.260 |
| | Kidney: Whole | 9.07^{†} | 5.98^{†} | 2.51 | 1.61 | 1.20 | 1.27 | 0.429 | 0.277 |
| | Urinary bladder contents | NS | 0.977 | 0.365 | NS | NS | 0.151 | 0.049 | 0.085 |
| | Urinary bladder wall | 0.841 | 0.907 | 1.10 | 0.433 | NS | 0.473 | 0.246 | 0.207 |
| Exocrine | Ex-orbital lachrymal gland | 0.686 | 0.613 | 0.774 | 0.766 | 0.659 | 1.01 | 0.539 | 0.281 |
| | Harderian gland | 2.59 | 2.74 | 2.68 | 2.08 | 0.471 | 0.388 | 0.181 | 0.105 |
| | Pancreas | 0.916 | 2.02 | 2.23 | 1.34 | 1.40 | 1.86 | 0.690 | 0.535 |
| | Preputial gland | 10.4^{†} | NS | 8.00^{†} | 8.17^{†} | 6.28^{†} | 7.40^{†} | 1.54 | 1.82 |
| | Salivary gland | 1.78 | 2.50 | 1.81 | 1.51 | 0.907 | 1.36 | 0.446 | 0.486 |
| Fatty | Fat: Brown | 0.618 | 8.67^{†} | 2.87 | 4.61 | 1.62 | 1.23 | 0.632 | 0.482 |
| | Fat: White | 0.063 | 0.466 | 0.890 | 0.492 | 1.15 | 1.68 | 0.847 | 1.33 |
| Ocular | Eye: Lens | 0.038 | 0.034 | 0.033 | 0.033 | 0.036 | 0.014 | 0.017 | 0.047 |
| | Eye: Whole | 0.353 | 0.116 | 0.112 | 0.057 | 0.076 | 0.070 | 0.030 | 0.059 |
| Reproductive | Epididymis | 2.93 | 1.57 | 1.10 | 1.31 | 0.951 | 0.751 | 0.358 | 0.211 |
| | Prostate gland | NS | 0.531 | 0.769 | NS | 0.222 | 0.431 | 0.218 | 0.107 |
| | Seminal vesicles | 1.01 | 1.33 | 1.36 | 1.03 | 0.527 | 0.656 | 0.372 | 0.250 |
| | Testis | 2.12 | 1.37 | 0.727 | 0.320 | 0.236 | 0.321 | 0.186 | 0.147 |
| Respiratory | Lung | 3.36 | 1.14 | 1.52 | 0.788 | 0.513 | 0.709 | 0.283 | 0.188 |
| Skeletal/ | Muscle (skeletal) | 0.432 | 0.318 | 0.383 | 0.424 | 0.626 | 0.707 | 0.370 | 0.462 |
| muscular | Myocardium | 1.19 | 2.63 | 2.66 | 1.52 | 1.47 | 2.49 | 1.36 | 1.53 |
| Vascular/ | Blood (cardiac) | 2.32 | 0.765 | 0.461 | 0.166 | 0.098 | 0.147 | 0.053 | 0.053 |
| lymphatic | Bone marrow | 2.76 | 2.36 | 2.06 | 1.20 | 0.695 | 0.784 | 0.204 | 0.144 |
| | Lymph duct | 3.45 | 1.13 | NS | 0.414 | 0.609 | 0.176 | 0.045 | 0.024 |
| | Lymph node | 1.69 | 1.66 | 1.40 | 0.869 | 0.550 | 0.712 | 0.270 | 0.081 |
| | Spleen | 2.09 | 2.08 | 1.75 | 1.01 | 0.789 | 0.865 | 0.234 | 0.134 |
| | Thymus | 1.52 | 1.56 | 1.31 | 0.815 | 0.362 | 0.502 | 0.208 | 0.110 |

^{†}Above limit of accurate quantification (>4.96 μg equivalents/g)
BLQ Below limit of accurate quantification (<0.004 μg equivalents/g)
NS No sample - tissue not sectioned

### Example 3: LPC Pharmacokinetics—Intravenous Administration

10 male Sprague Dawley rats, in the weight range of 229-286 g and approximately 7-8 weeks old at the time of dose administration were housed in polypropylene cages and remained therein except for a short period during dosing. The room in which the animals were located was thermostatically monitored and data recorded continually (generally the temperature range was 21±2° C.; humidity range 55±10%) and exposed to 12 hours fluorescent lighting and 12 hours dark per day. Animals were equilibrated under standard animal house conditions for a minimum of 3 days prior to use. The health status of the animals was monitored throughout this period and the suitability of each animal for experimental use was confirmed before use.

A pellet diet (RM1 (E) SQC, Special Diets Services, Witham, Essex, UK) and water (from the domestic water supply) was available ad libitum throughout the holding, acclimatization and post-dose periods.

The 10 male Sprague Dawley rats (two groups of five) each received a single intravenous administration of either formulation A or formulation B (five per formulation) according to the dosage specification specified in example 1. Each rat was weighed prior to dose administration and the individual doses administered were calculated based on the bodyweight and the specified dose volume.

Dose utensils for intravenous administration consisted of a hypodermic syringe and needle. The dose was administered directly into a tail vein as a slow bolus over 30 seconds.

Serial samples of whole blood (each approximately 0.15 mL in the first 24 hours and approximately 0.21 mL in subsequent samples) were collected via a tail vein from each animal at: 0.2, 0.5, 0.75, 1, 2, 3, 4, 6, 8, 12, 24, 30, 48, 72 and 96 hours post-dose. A terminal whole blood sample (approximately 6 to 8 mL) was obtained from each animal via cardiac puncture under isoflurane anesthesia at 168 hours post-dose. Animals were killed by cervical dislocation after the final blood collection.

Whole blood was collected into tubes containing lithium heparin as anticoagulant. As soon as practicable after collection, samples were centrifuged (at approximately 3000 G at +4° C. for 10 minutes) and the resultant plasma was removed into plain tubes and blood cells discarded. Any residual plasma samples were stored at approximately −20° C.

For the pharmacokinetics investigations, individual concentration data for radioactivity in plasma were entered into PCModfit v4.0. Relevant pharmacokinetic parameters were derived using non compartmental analysis (linear/logarithmic trapezoidal). The pharmacokinetic parameters calculated (where appropriate) were:
Cmax Maximum observed concentration
tmax Time point at which Cmax was observed
t½ Half-life for the terminal elimination phase
AUC0-t Area under the concentration versus time curve from time 0 to the final sampling time
AUC0-inf Area under the concentration versus time curve from time 0 extrapolated to infinite time.

For the male Sprague Dawley rats selected for the pharmacokinetic study which received a single intravenous dose of [¹⁴C]-LPC-EPA at a target dose level of 1.55 mg/kg (radioactive dose ca. 1.5 Ki/rat) the maximum mean concentration of total radioactivity in plasma (10.6 μg.equiv/g) occurred 0 h post dose administration).

Total radioactivity concentrations declined thereafter and were detectable at the final sampling time (0.0403 μg.equiv/g; 168 hours).

Blood concentrations achieved a maximum mean concentration of total radioactivity (6.15 μg.equiv/g) at 0 hours post dose administration. Total radioactivity concentrations declined thereafter and were detectable at the final sampling time (0.0840 μg.equiv/g; 168 hours).

Pharmacokinetic parameters of total radioactivity measured in plasma and whole blood following a single intravenous administration of [¹⁴C]-LPC-EPA to male Sprague Dawley rats at a mean dose of 1.5 mg/kg in intralipid (190 mg/mL) is shown in table 3a and 3b respectively.

TABLE 3a
Plasma

| Parameter | Mean |
|---|---|
| Cmax | 10.6 |
| (μg.equiv/g) | |
| Tₘₐₓ (h) | 0 |
| T½ (h) | 57.6 |
| AUC₀₋ₜ | 34.7 |
| (μg.h/mL) | |
| AUC_{0-inf} | 38.0 |
| (μg.h/mL) | |

TABLE 3b
Blood

| Parameter | Mean |
|---|---|
| Cmax | 6.15 |
| (μg.equiv/g) | |
| Tₘₐₓ (h) | 0 |
| T½ (h) | 115 |
| AUC₀₋ₜ | 29.4 |
| (μg.h/mL) | |
| AUC_{0-inf} | 43.3 |
| (μg.h/mL) | |

For the male Sprague Dawley rats selected for the pharmacokinetic study that received a single intravenous dose of [¹⁴C]-LPC-DHA at a target dose level of 1.55 mg/kg (radioactive dose ca. 1.5 Ki/rat) the maximum mean concentration of total radioactivity in plasma (5.08 μg.equiv/g) occurred 0 h post dose administration. Total radioactivity concentrations declined thereafter and were detectable at the final sampling time (0.0611 μg.equiv/g; 168 hours).

Blood concentrations achieved a maximum mean concentration of total radioactivity (2.46 μg.equiv/g) at 0 hours post dose administration. Total radioactivity concentrations declined thereafter and were detectable at the final sampling time (0.115 μg.equiv/g; 168 hours).

Pharmacokinetic parameters of total radioactivity measured in plasma and whole blood following a single intravenous administration of [¹⁴C]-LPC-DHA to male Sprague Dawley rats at a mean dose of 1.5 mg/kg in intralipid (190 mg/mL) is shown in table 4a and 4b respectively.

TABLE 4a
Plasma

| Parameter | Mean |
|---|---|
| Cmax | 5.08 |
| (μg.equiv/g) | |
| Tₘₐₓ (h) | 0 |
| T½ (h) | 61.3 |
| AUC₀₋ₜ | 40.8 |
| (μg.h/mL) | |
| AUC_{0-inf} | 46.3 |
| (μg.h/mL) | |

TABLE 4b
Blood

| Parameter | Mean |
|---|---|
| Cmax | 2.46 |
| (μg.equiv/g) | |
| Tₘₐₓ (h) | 0 |
| T½ (h) | 159 |
| AUC₀₋ₜ | 35.1 |
| (μg.h/mL) | |
| AUC_{0-inf} | 61.4 |
| (μg.h/mL) | |

### Example 4: LPC Pharmacokinetic Modelling—Intravenous Administration Compared to Oral Administration

Intravenously administered [¹⁴C]-LPC-DHA and [¹⁴C]-LPC-EPA was further analyzed by compartmental pharmacokinetic modelling. The plasma and blood concentration-time curves were described by a three-compartment terminal distribution and elimination model with up to three recycling compartments all directly linked to the central (plasma) compartment. The general structure of the model is shown in FIG. 9.

To allow for time dependent out- and in-flow the recycling compartments were modelled with Heaviside continuous step functions. The full matrix of the terminal (separated from the recycling model) distribution model is given in the panel below.

$$\frac{\partial q 2}{\partial t} = q 3 \cdot \text{?} + q 4 \cdot {k}_{( 2 , 4 )} + q 1 \cdot {k}_{( 2 , 1 )} + q 5 \cdot {k}_{( 2 , 5 )} + q 6 \cdot {k}_{( 2 , 6 )} - q 2 \cdot [ \text{?} + {k}_{( 4 , 2 )} + {k}_{( 3 , 2 )} + {k}_{( 1 , 2 )} + {k}_{( 5 , 2 )} + {k}_{( 6 , 2 )} ]$$

$$\frac{\partial q 3}{\partial t} = q 2 \cdot {k}_{( 3 , 2 )} - q 3 \cdot {k}_{( 2 , 3 )}$$

$$\frac{\partial q 4}{\partial t} = q 2 \cdot {k}_{( 4 , 2 )} - q 4 \cdot {k}_{( 2 , 4 )}$$

$$\frac{\partial q 5}{\partial t} = q 2 \cdot {k}_{( 5 , 2 )} - q 5 \cdot {k}_{( 2 , 5 )}$$

$$\frac{\partial q 6}{\partial t} = q 2 \cdot {k}_{( 6 , 2 )} - q 6 \cdot {k}_{( 2 , 6 )}$$

$$The bolus dose D is presented into compartment q 2$$

$$\text{?} \text{indicates text missing or illegible when filed}$$

To allow simultaneous on-off of in vs outflow of the loss-less recycling out and into the central (plasma) compartment were regulated by 3 Heaviside functions set up in parallel with the same function inversed as shown in the panel below.

$${k}_{( 5 , 2 )} = {ko}_{1} [ 0.5 ( 1 + arctan ( L ( {t}_{lag 1} - t ) ) \cdot \frac{2}{π} ] {k}_{( 2 , 5 )} = {kl}_{1} [ 0.5 ( 1 + arctan ( L ( t - {t}_{lag 1} ) ) \cdot \frac{2}{π} ]$$

$${k}_{( 6 , 2 )} = {ko}_{2} [ 0.5 ( 1 + arctan ( L ( {t}_{lag 2} - t ) ) \cdot \frac{2}{π} ] {k}_{( 2 , 5 )} = {kl}_{2} [ 0.5 ( 1 + arctan ( L ( t - {t}_{lag 2} ) ) \cdot \frac{2}{π} ]$$

$$\text{?} = {ko}_{3} [ 0.5 ( 1 + arctan ( L ( {t}_{lag 3} - t ) ) \cdot \frac{2}{π} ] \text{?} = {kl}_{3} [ 0.5 ( 1 + arctan ( L ( t - {t}_{lag 3} ) ) \cdot \frac{2}{π} ]$$

$$Individual compartments can be made void by setting the outflow and corresponding inflow = 0$$

$$\text{?} \text{indicates text missing or illegible when filed}$$

Since the flow through the step function controlled are loss less and confined to a semi-discrete time interval ahead of the terminal distribution and elimination phases the regular distribution and elimination micro constants (as relates to compartments q2, q3 and q4) can be solved for the corresponding macro constants: 2 distribution constants, lambda2 and lambda3 and the terminal elimination constant lambda1 from which the corresponding half-lives can be calculated.

Arranging the rate (micro) constants of the systems such that:

$${a}_{0} = {k}_{( 0 , 2 )} \cdot {k}_{( 2 , 3 )} \cdot {k}_{( 2 , 4 )}$$

$${a}_{1} = ( {k}_{( 0 , 2 )} \cdot {k}_{( 2 , 4 )} ) + ( {k}_{( 2 , 3 )} \cdot {k}_{( 2 , 4 )} ) + ( {k}_{( 2 , 3 )} \cdot {k}_{( 4 , 2 )} ) + ( {k}_{( 0 , 2 )} \cdot {k}_{( 2 , 3 )} ) + ( {k}_{( 2 , 4 )} \cdot {k}_{( 3 , 2 )} )$$

$${a}_{2} = {k}_{( 0 , 2 )} + {k}_{( 2 , 3 )} + {k}_{( 2 , 4 )} + {k}_{( 3 , 2 )} + {k}_{( 4 , 2 )}$$

$$then let :$$

$${p}_{1} = {a}_{1} - ( \text{?} ) \text{?} = ( \text{?} ) - ( \text{?} ) + {a}_{0}$$

$${r}_{1} = \sqrt{- \text{?}} {r}_{2} = 2 \cdot \text{?} {r}_{3} = \frac{arccos ( \text{?} )}{3}$$

$$\text{?} \text{indicates text missing or illegible when filed}$$

This cubic can be solved to yield the rate macro constants and their

$$\begin{matrix}{λ}_{1} = - [ cos ( {r}_{3} ) \cdot {r}_{2} - ( \text{?} ) ] & \text{?} {λ}_{1} = \frac{ln ( 2 )}{\text{?}} \\ {λ}_{2} = - [ cos ( {r}_{3} + \frac{2 π}{3} ) \cdot {r}_{2} - ( \text{?} ) ] & \text{?} {λ}_{2} = \frac{ln ( 2 )}{\text{?}} \\ {λ}_{3} = - [ cos ( {r}_{3} + \frac{4 π}{3} ) \cdot {r}_{2} - ( \text{?} ) ] & \text{?} {λ}_{3} = \frac{ln ( 2 )}{\text{?}}\end{matrix}$$

$$\text{?} \text{indicates text missing or illegible when filed}$$

System volumes and clearances defined by the micro constants:

$$\begin{matrix}\text{?} = {k}_{( 0 , 2 )} \cdot {V}_{c} \\ {CL}_{3} = {k}_{( 3 , 2 )} \cdot {V}_{c} = {k}_{( 2 , 3 )} \cdot {V}_{3} \\ {CL}_{4} = {k}_{( 4 , 2 )} \cdot {V}_{c} = {k}_{( 2 , 4 )} \cdot {V}_{4}\end{matrix} {V}_{3} = \text{?} \cdot {V}_{c} {V}_{4} = \frac{{k}_{( 4 , 2 )}}{{k}_{( 2 , 4 )}} \cdot {V}_{c} \text{?} = {V}_{3} + {V}_{c}$$

$$\text{?} \text{indicates text missing or illegible when filed}$$

The compartmental modelling software SAAM II version 2.3.1.1 (University of Washington and The Epsilon Group) was used to set up and solve this model. A similar model but with a model of lag regulate uptake from the gut, was established to describe orally administered [¹⁴C]-LPC-DHA and [¹⁴C]-LPC-EPA. The result demonstrate a very similar kinetic that is independent of the mode of administration.

The plasma and blood profiles of [¹⁴C]-LPC-DHA and [¹⁴C]-LPC-EPA obtained from animal experiments as described in Example 2, exhibits some very unusual properties, in particular the rapid and extensive recirculation of mass from the central compartment. Model parameter estimates are given in the panel below together with key parameters from non-compartmental statistical analysis: The panel also contains similar data for [¹⁴C]-PC-DHA and [¹⁴C]-PC-EPA for comparison. (PC=phosphatidylcholine).

| Non-compartmental | Oral | | | | | I.V. | |
|---|---|---|---|---|---|---|---|
| Parameter | units | EP A-PC | EPA-LPC | DHA-PC | DHA-LPC | EPA-LPC | DHA-LPC |
| Does of 14C-FA | mg/kg | 3.413 | 1.681 | 3.719 | 1.694 | 1.522 | 1.520 |
| AUC 0-t | μg/g*hrs | 51.7 | 34.0 | 41.7 | 34.4 | 34.2 | 40.9 |
| AUC t-inf | μg/g*hrs | 6.38 | 3.69 | 7.19 | 5.18 | 3.34 | 5.42 |
| AUC 0-inf | μg/g*hrs | 58.1 | 37.8 | 48.9 | 39.5 | 37.5 | 46.3 |
| ke | 1/h | 0.0114 | 0.0115 | 0.0108 | 0.0121 | 0.0121 | 0.0113 |
| t/2 | h | 60.6 | 60.14 | 63.9 | 57.4 | 57.3 | 61.4 |
| F | % | na | 91.1 | na | 76.6 | 100 | 100 |
| Cl tot (ml/(kg*hrs)) | mL/(g BW*h) | na | na | na | na | 40.6 | 32.8 |
| Vdβ (ml/kg) | ml/kg | na | na | na | na | 3356 | 2908 |
| AUMC 0-INF | μg equiv./g*hrs² | 3882.5 | 2311.8 | 4027.9 | 2995.5 | 2200 | 3266 |
| (μg equiv./g*hrs²) | | | | | | | |
| MRT (hrs) | hrs | 66.8 | 61.2 | 82.4 | 75.7 | 59 | 70 |
| Compartmental | | | | | | | |
| model Parameter | EP A-PC | | EPA-LPC | DHA-PC | DHA-LPC | EPA-LPC | DHA-LPC |
| Vc | mL/g BW | 1.16500 | 0.77396 | 1.07400 | 0.68477 | 0.13212 | 0.25662 |
| Kk(0, 2) | 1/hrs | 0.04700 | 0.07545 | 0.06900 | 0.06421 | 0.31069 | 0.14516 |
| k(2, 3) | 1/hrs | 0.01700 | 0.00970 | 0.01400 | 0.02007 | 0.01200 | 0.01287 |
| k(2, 4) | 1/hrs | 0.03850 | 0.03802 | 0.05600 | 0.06183 | 0.03717 | 0.0269 |
| k(3, 2) | 1/hrs | 0.00650 | 0.01164 | 0.00900 | 0.02893 | 0.01708 | 0.01612 |
| k(4, 2) | 1/hrs | 0.07800 | 0.08870 | 0.22500 | 0.18635 | 0.42476 | 0.15812 |
| k(5, 1) | 1/hrs | 2.88600 | na | 1.13300 | na | na | na |
| k(6, 1) | 1/hrs | 0.17600 | na | 1.32600 | na | na | na |
| ka | 1/hrs | 1.07200 | 2.42534 | 5.53100 | 1.3376 | na | na |
| ka2 | 1/hrs | na | 0.71022 | na | 0.7892 | na | na |
| ka3 | 1/hrs | na | 0.17149 | na | 0.2241 | na | na |
| kl1 | 1/hrs | na | na | na | na | 2.67408 | 3.16003 |
| kl2 | 1/hrs | na | na | na | na | 0.54396 | 0.70964 |
| kl3 | 1/hrs | na | na | na | na | na | 0.10254 |
| ko1 | 1/hrs | na | na | na | na | 0.40081 | 4.82757 |
| ko2 | 1/hrs | na | na | na | na | 0.22384 | 0.59763 |
| ko3 | 1/hrs | na | na | na | na | 0.00000 | 0.35856 |
| HS-coeff1(L1) | na | na | 32 | na | 28.08 | 30 | 4 |
| HS-coeff2(L2) | na | na | 23 | na | 62.40 | 26 | 26 |
| HS-coeff3(L3) | na | na | 1 | na | 0.74000 | na | 1 |
| tlag | hrs | 0.48 | 0.90 | 0.93 | 0.87 | 0.78 | 1.22 |
| tlag2 | hrs | 1.02 | 3.69 | 1.93 | 3.25 | 5.59 | 4.51 |
| tlag3 | hrs | 23.90 | 15.97 | 27.83 | 27.97 | na | 22.23 |
| Derived COMP | | | | | | | |
| model Parameter | EPA-PC | | EPA-LPC | DHA-PC | DHA-LPC | EPA-LPC | DHA-LPC |
| Vb | mL/g BW | 1.74600 | 1.65365 | 1.74900 | 1.67076 | 0.30827 | 0.56506 |
| V3 | mL/g BW | 0.58100 | 0.87969 | 0.67600 | 0.98599 | 0.17615 | 0.30844 |
| V4 | mL/g BW | 2.36400 | 1.33755 | 4.34300 | 1.93213 | 1.64764 | 1.38958 |
| CLO | mL/(g BW*h) | 0.05490 | 0.04376 | 0.07430 | 0.04096 | 0.03739 | 0.03506 |
| CL3 | mL/(g BW*h) | 0.00990 | 0.00851 | 0.00960 | 0.02024 | 0.00230 | 0.00394 |
| CL4 | mL/(g BW*h) | 0.09090 | 0.05189 | 0.24180 | 0.11686 | 0.05587 | 0.03757 |
| λ1 | 1/hrs | 0.00967 | 0.00732 | 0.00911 | 0.00866 | 0.01025 | 0.00954 |
| λ2 | 1/hrs | 0.18907 | 0.19822 | 0.34671 | 0.32440 | 0.77637 | 0.33384 |
| λ3 | 1/hrs | 0.98591 | 0.01797 | 0.01729 | 0.02834 | 0.01573 | 0.01580 |
| t 1/2 λ1 | 1/hrs | 71.7 | 98.5 | 76.1 | 80.7 | 68.9 | 73.1 |
| t 1/2 λ2 | 1/hrs | 4.4 | 4.70 | 2.0 | 2.4 | 0.9 | 2.3 |
| t 1/2 λ3 | 1/hrs | 34.8 | 40.3 | 40.1 | 25.5 | 47.3 | 44.0 |
| AUCO-t | μg/g*hrs | na | 34.2 | na | 34.6 | 35.5 | 41.8 |
| Plasma | | | | | | | |
| AUCO-inf | μg/g*hrs | na | 39.00 | na | 41.8 | 41.0 | 47.9 |
| Plasma | | | | | | | |
| Vd area/F | μg/g*hrs | na | 6151.8 | na | 4782.7 | na | na |
| Vd area | ml/kg | na | na | na | na | 3802.6 | 3736.7 |

The data and models shown in panels above demonstrate a clear and consistent commonality in their kinetics. The primary kinetic determinants for the PC forms are surprisingly similar between the two fatty acids.

Targeted and controlled dosing to deep tissues:

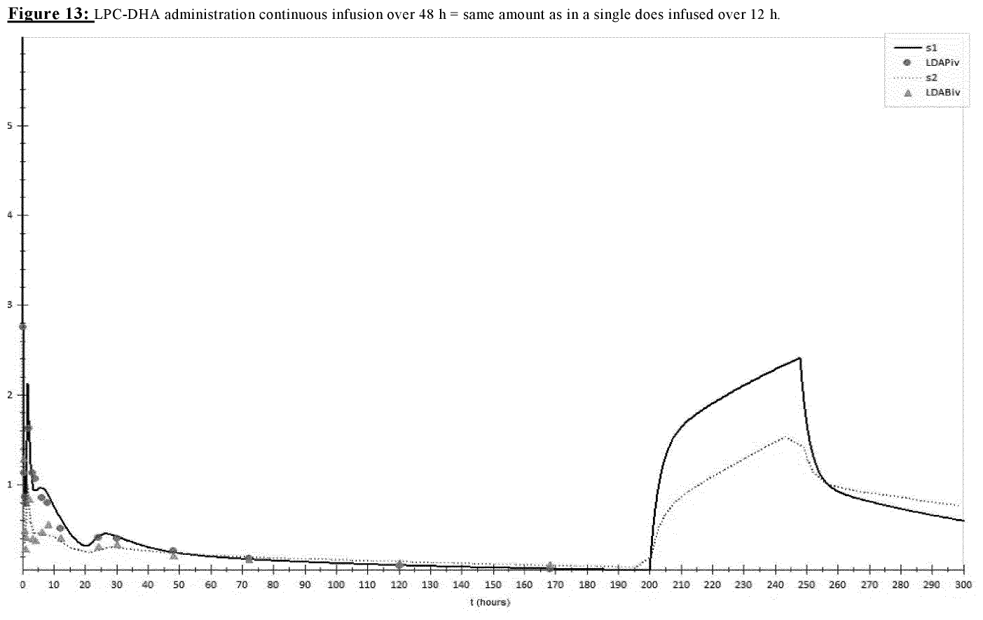

While orally administered [¹⁴C]-LPC-DHA [¹⁴C]-LPC-EPA, [¹⁴C]-PC-DHA, and [¹⁴C]-PC-EPA all demonstrate clear and consistent fluctuations during uptake and early distribution, the plasma concentration-time data and compartmental model reveal a clear difference in the fluctuations seen during the first 24 hours after ingestion with the LPC showing the most fluctuations. However, the clearest difference is between i.v. [¹⁴C]-LPC-DHA and [¹⁴C]-LPC-EPA and the other forms. Such rapid fluctuations in the concentration-time curve indicates that the injected LPC-EPA and LPC DHA are very rapidly taken up by certain organs, in particular those that are very well transfused and that also carries the EPA-LPC DHA-LPC transporter Mfsd2a, such as the brain, the, ocular organs, the liver and the intestinal mucosa. The ability of the compartmental model to faithfully describe the observed fluctuations makes it possible to use it for simulation of the effects of other dosing regimens. FIG. 13 is a graphical representation of the data and model simulated use of a long-term constant infusion rather than a bolus injection. The simulation suggests that highly transfused tissues such as the brain might be supplied a constant and well titrated dose LPC bound EPA and DHA. As such this provides a means by which one can supply such deep tissues (brain, spleen, retina, intestinal mucosa, bone mallow, leucocytes, liver, reproductive organs, skin (list not exhaustive)) with therapeutic concentrations of EPA and DHA. FIG. 13 is a demonstration of how a careful (same amount as in single bolus dose infused over 12 h) continuous infusion over 48 h of LPC-DHA can create a long-lasting stable plateau that still does not produce the same very sharp spike as the bolus. The bolus is given at time 0 while the infusion is started at 200 h and lasts until 248 h.

### Example 5: Effects of Composition Comprising a Combination of LPC EPA and LPC DHA

This example provides data from three weeks daily dosing with different krill oil lysophospholipid compositions containing LPC-EPA and LPC-DHA. It was of interest to investigate whether krill oil lysophospholipid compositions cause an increase in plasma LPC-DHA/EPA and increase whole brain EPA and DHA content. The EPA, DHA and total omega-3 contents of these oils are given in Table 5 below. Krill oil lysophospholipid compositions of various purities and production of these have previously been described in detail (WO2019/123015).

TABLE 5
EPA, DHA and total omega-3 contents in test products.

| Batch A100588- | FA contents in oil (g/100 g) | | | Phospholipid-bound | |
|---|---|---|---|---|---|
| 20190924 | EPA | DHA | Total Omega-3 | EPA | DHA |
| Olive oil | 0 | 0 | 0 | 0 | 0 |
| Boost | 17.3 | 10.0 | 35.2 | 11.1 | 6.3 |
| Crude (27% LPC) | 16.9 | 9.9 | 34.7 | 10.6 | 6.7 |
| Pure (89% LPC) | 23.7 | 11.7 | 44.0 | 23.2 | 11.3 |

Twenty-four male rats were divided into six groups and received daily oral gavage for 3 weeks, containing: Group 1) Olive oil (0 mg/kg/day EPA and 0 mg/kg/day DHA); Group 2) Crude (27% LPC), low dose (185 mg/kg/day EPA and 108 mg/kg/day DHA), 3) Crude (27% LPC), medium dose (370 mg/kg/day EPA and 217 mg/kg/day DHA), 4) Crude (27% LPC), high dose (926 mg/kg/day EPA and 543 mg/kg/day DHA), 5) Pure (89% LPC), medium dose (324 mg/kg/day EPA and 160 mg/kg/day DHA), 6) Superba Boost krill oil, medium dose (379 mg/kg/day EPA and 219 mg/kg/day DHA).

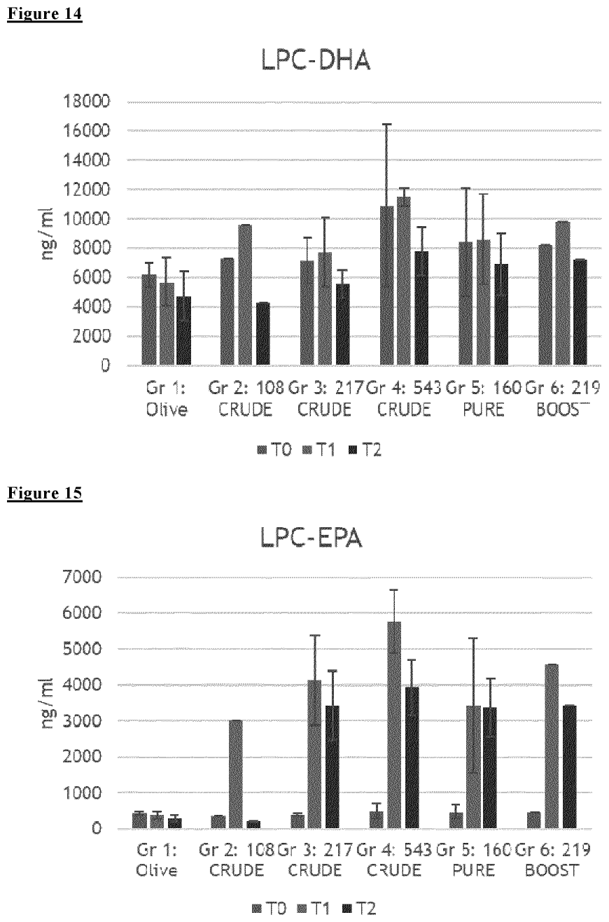

LPC-DHA and LPC-EPA was extracted from plasma using a Bligh and Dyer protocol and dissolved in ethanol prior to LC-MS/MS analysis. Samples were collected at baseline (T0), after 10 days of oral gavage (T1) and after 22 days of oral gavage (T2). The results are displayed in FIGS. 14 and 15. Rats fed with krill oil lysophospholipid compositions showed an increase in plasma LPC-DHA at T1 and LPC-EPA at T1 and T2 relative to the olive oil group, as well as a dose-dependent increase in plasma LPC-EPA. In general, an elevation in LPC-EPA/DHA plasma content indicates that more EPA and DHA is available for brain uptake via mfsd2a.

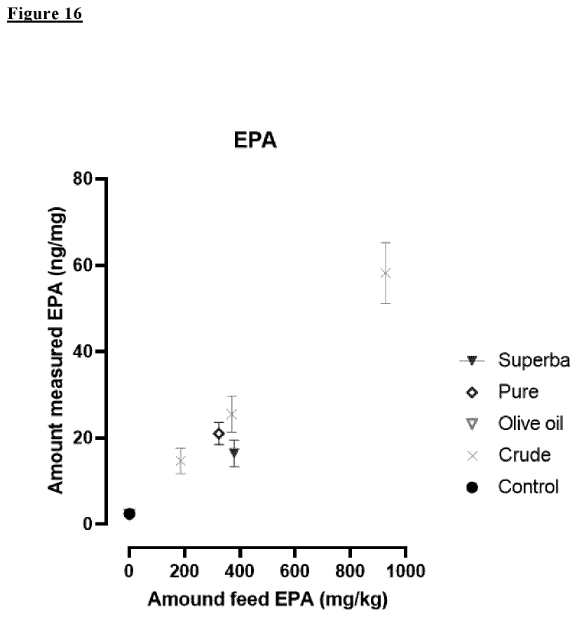

Following homogenization and freeze drying of brain tissue, whole brain FAs were extracted with Bligh and Dyer, hydrolyzed and analyzed by HPLC. As can be seen in FIG. 16, there is a very strong dose-response relationship, with higher doses of EPA being associated with higher brain EPA concentrations (ng/mg). Surprisingly, there was no difference in brain EPA concentration between the “crude” and “pure” krill oil lysophospholipid compositions at similar doses. However, rats fed with krill oil lysophospholipid compositions shows higher brain EPA concentrations than rats fed with Superba Boost krill oil at similar doses. This implies that brain EPA uptake from the two krill oil lysophospholipid compositions is superior relative to Superba Boost krill oil, and this is due to the relatively higher LPC-EPA content in the krill oil lysophospholipid compositions.

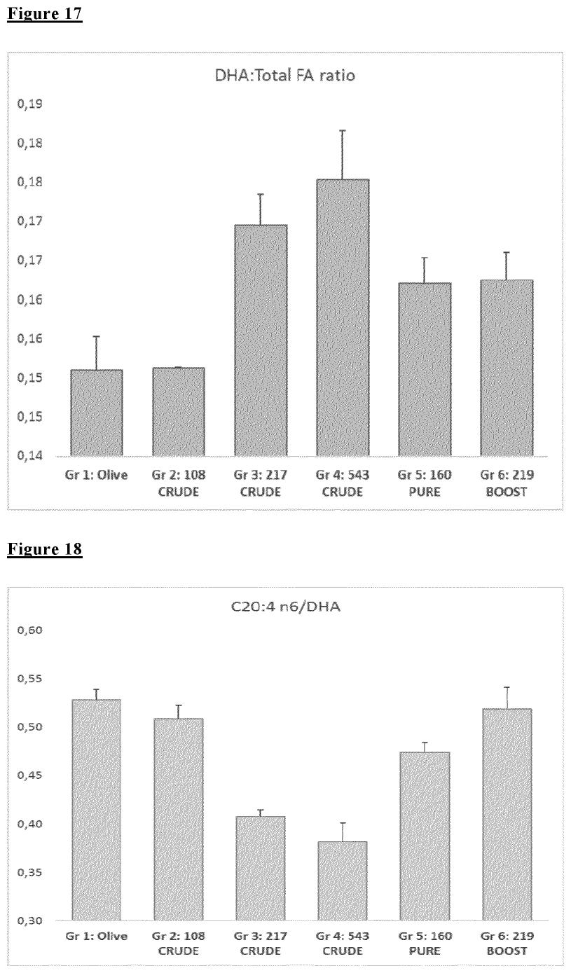

Analysis of Fatty Acid Methyl Esters (FAME) by GC-FID was used to assess brain DHA concentration in relation to total fatty acids (FIG. 17) and Arachidonic acid (ARA; 20:4 n-6) (FIG. 18). The results show that DHA:total FA ratio is dose-dependent, with the high dose Crude oil showing the highest ratio, followed by the medium dose Crude oil. At a medium dose, rats that were given the Crude oil showed a higher DHA:total FA ratio than the Superba Boost krill oil, and this is likely due to the higher amounts of LPC-DHA and LPC-EPA (and subsequent increased DHA/EPA uptake via mfsd2a) in the lysophospholipid product as compared to the Superba Boost krill oil. Similarly, FIG. 18 again shows a more favorable brain fatty acid profile for rats given the lysophospholipid compositions. Here, there is a dose-dependent decrease in the ARA:DHA ratio, with the high dose Crude oil having the lowest ratio. Furthermore, when comparing similar doses of Crude oil and Superba Boost oil the data shows a greater reduction of the ARA:DHA ratio for the Crude lysophospholipid composition relative to Superba Boost krill oil.

1-9. (canceled)

10. A method of therapy of traumatic brain injury in a subject in need thereof comprising administering by intravascular administration to the subject a pharmaceutical composition comprising a compound selected from the group of formulas 1 to 8 and combinations thereof: 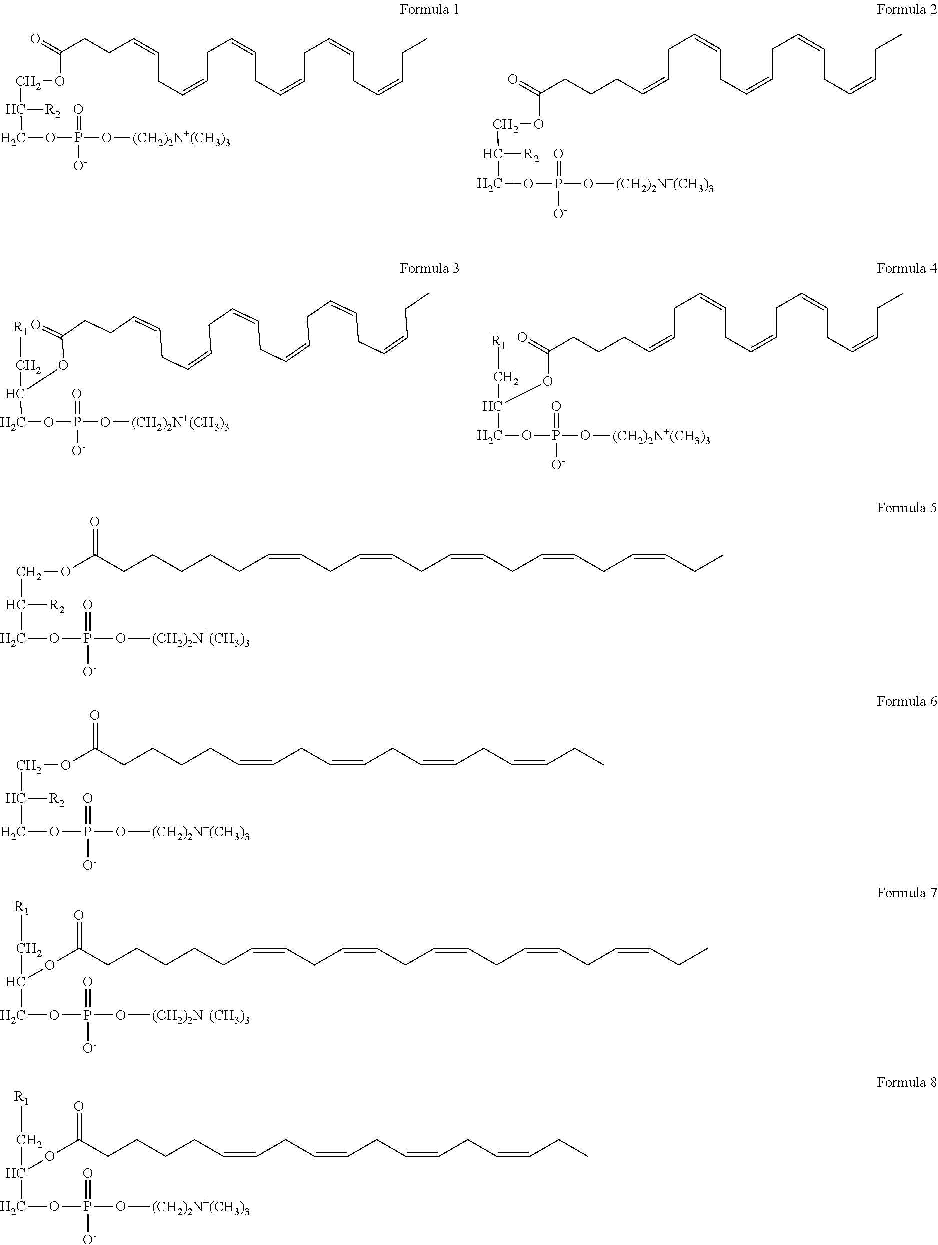
wherein R₁ is —OH and R₂ is —OH.

11. The method of claim 10, wherein the compound is an LPC-DHA selected from the group consisting of: 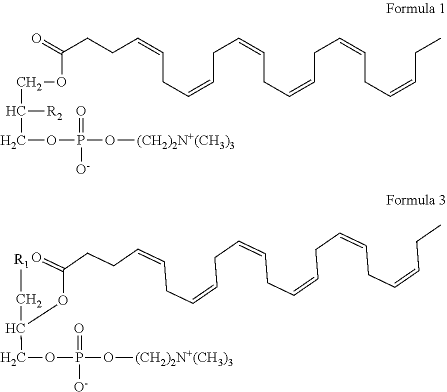
and combinations thereof.

12. (canceled)

13. The method according to claim 10, wherein the intravascular administration is intravenous administration.

14-16. (canceled)

17. The method according to claim 11, wherein the LPC-DHA constitutes from 10 to 99% by weight of the pharmaceutical composition.

18. The method according to claim 11, wherein the LPC-DHA constitutes from 20 to 99% by weight of the pharmaceutical composition.

19. The method according to claim 10, wherein the LPC-DHA constitutes from 50 to 99% by weight of the pharmaceutical composition.

20. The method according to claim 11, wherein the LPC-DHA constitutes from 70 to 99% by weight of the pharmaceutical composition.

21-25. (canceled)

26. The method of claim 11, wherein the pharmaceutical composition further comprises LPC-EPA and the molar ratio of lysoPC-EPA:lysoPC-DHA is in the range 1:1 to 5:1; with the proviso that i) the number of moles of lysoPC-EPA is the number of moles 1-lysoPC-EPA+the number of moles 2-lysoPC-EPA; and ii) the number of moles of lysoPC-DHA is the number of moles 1-lysoPC-DHA+the number of moles 2-lysoPC-DHA.
